SEQUENCE LISTING (869 sequences)

SEQ ID NO: 1 - DNA, 10 bp, Homo sapiens
aaaccattct

SEQ ID NO: 2 - DNA, 10 bp, Homo sapiens
aaggcaggga

SEQ ID NO: 3 - DNA, 10 bp, Homo sapiens
acacagcaag

SEQ ID NO: 4 - DNA, 10 bp, Homo sapiens
agctggagtc

SEQ ID NO: 5 - DNA, 10 bp, Homo sapiens
agctggcacc

SEQ ID NO: 6 - DNA, 10 bp, Homo sapiens
ataaatgagg

SEQ ID NO: 7 - DNA, 10 bp, Homo sapiens
caagcacccc

SEQ ID NO: 8 - DNA, 10 bp, Homo sapiens
cactacccac

SEQ ID NO: 9 - DNA, 10 bp, Homo sapiens
cactactcac

SEQ ID NO: 10 - DNA, 10 bp, Homo sapiens
cccacctcca

SEQ ID NO: 11 - DNA, 10 bp, Homo sapiens
cccgcctctt

SEQ ID NO: 12 - DNA, 10 bp, Homo sapiens
cctcagatgt

SEQ ID NO: 13 - DNA, 10 bp, Homo sapiens
cgctactcac

SEQ ID NO: 14 - DNA, 10 bp, Homo sapiens
ctaagacctc

SEQ ID NO: 15 - DNA, 10 bp, Homo sapiens
ctaagacttc

SEQ ID NO: 16 - DNA, 10 bp, Homo sapiens
gagtgggtgc

SEQ ID NO: 17 - DNA, 10 bp, Homo sapiens
gggacagctg

SEQ ID NO: 18 - DNA, 10 bp, Homo sapiens
gggttggctt

SEQ ID NO: 19 - DNA, 10 bp, Homo sapiens
gtaagtgtac

SEQ ID NO: 20 - DNA, 10 bp, Homo sapiens
gtaagtgtac

SEQ ID NO: 21 - DNA, 10 bp, Homo sapiens
gtaggggtaa

SEQ ID NO: 22 - DNA, 10 bp, Homo sapiens
taaccactgc

SEQ ID NO: 23 - DNA, 10 bp, Homo sapiens
tactgctcgg

SEQ ID NO: 24 - DNA, 10 bp, Homo sapiens
tcaggctgaa

SEQ ID NO: 25 - DNA, 10 bp, Homo sapiens
tccatacacc

SEQ ID NO: 26 - DNA, 10 bp, Homo sapiens
tccttttaaa

SEQ ID NO: 27 - DNA, 10 bp, Homo sapiens
tgattaaggt

SEQ ID NO: 28 - DNA, 10 bp, Homo sapiens
tggtatcaca

SEQ ID NO: 29 - DNA, 10 bp, Homo sapiens
tggtgtatgc

SEQ ID NO: 30 - DNA, 10 bp, Homo sapiens
tgtcactggg

SEQ ID NO: 31 - DNA, 10 bp, Homo sapiens
tgtgggaggc

SEQ ID NO: 32 - DNA, 10 bp, Homo sapiens
tttaacggcc

SEQ ID NO: 33 - DNA, 10 bp, Homo sapiens
gctctctatg

SEQ ID NO: 34 - DNA, 10 bp, Homo sapiens
agaatgaaac

SEQ ID NO: 35 - DNA, 10 bp, Homo sapiens
aagtggaata

SEQ ID NO: 36 - DNA, 10 bp, Homo sapiens
gatgacgact

SEQ ID NO: 37 - DNA, 10 bp, Homo sapiens
ccctttcaca

SEQ ID NO: 38 - DNA, 10 bp, Homo sapiens
tcctggggca

SEQ ID NO: 39 - DNA, 10 bp, Homo sapiens
tctattgatg

SEQ ID NO: 40 - DNA, 10 bp, Homo sapiens
ggggctgtat

SEQ ID NO: 41 - DNA, 10 bp, Homo sapiens
cccaggacac

SEQ ID NO: 42 - DNA, 10 bp, Homo sapiens
ggagctgctg

SEQ ID NO: 43 - DNA, 10 bp, Homo sapiens
tggacagcag

SEQ ID NO: 44 - DNA, 10 bp, Homo sapiens
tctgggaaca

SEQ ID NO: 45 - DNA, 10 bp, Homo sapiens
cctgtgtatg

SEQ ID NO: 46 - DNA, 10 bp, Homo sapiens
ggcaagaaga

SEQ ID NO: 47 - DNA, 10 bp, Homo sapiens
aaatgcttgg

SEQ ID NO: 48 - DNA, 10 bp, Homo sapiens
ctaaaaacct

SEQ ID NO: 49 - DNA, 10 bp, Homo sapiens
gagcattgca

SEQ ID NO: 50 - DNA, 10 bp, Homo sapiens
ggtggacacg

SEQ ID NO: 51 - DNA, 10 bp, Homo sapiens
gctcctgagc

SEQ ID NO: 52 - DNA, 10 bp, Homo sapiens
aagaagtgga

SEQ ID NO: 53 - DNA, 10 bp, Homo sapiens
tgggaagtgg

SEQ ID NO: 54 - DNA, 10 bp, Homo sapiens
actcgctctg

SEQ ID NO: 55 - DNA, 10 bp, Homo sapiens
tttcagggga

SEQ ID NO: 56 - DNA, 10 bp, Homo sapiens
acaacgtcca

SEQ ID NO: 57 - DNA, 10 bp, Homo sapiens
gtctcagtgc

SEQ ID NO: 58 - DNA, 10 bp, Homo sapiens
ccccctgccc

SEQ ID NO: 59 - DNA, 10 bp, Homo sapiens
agaaaccacg

SEQ ID NO: 60 - DNA, 10 bp, Homo sapiens
gaccgcagga

SEQ ID NO: 61 - DNA, 10 bp, Homo sapiens
gtgctacttc

SEQ ID NO: 62 - DNA, 10 bp, Homo sapiens
gataactaca

SEQ ID NO: 63 - DNA, 10 bp, Homo sapiens
tggctgtgac

SEQ ID NO: 64 - DNA, 10 bp, Homo sapiens
gagtgagacc

SEQ ID NO: 65 - DNA, 10 bp, Homo sapiens
gagtggctac

SEQ ID NO: 66 - DNA, 10 bp, Homo sapiens
gactcaggga

SEQ ID NO: 67 - DNA, 10 bp, Homo sapiens
gttatatgcc

SEQ ID NO: 68 - DNA, 10 bp, Homo sapiens
gaggcgctgc

SEQ ID NO: 69 - DNA, 10 bp, Homo sapiens
gagctctgag

SEQ ID NO: 70 - DNA, 10 bp, Homo sapiens
gccagccagt

SEQ ID NO: 71 - DNA, 10 bp, Homo sapiens
atggcaacag

SEQ ID NO: 72 - DNA, 10 bp, Homo sapiens
aaggagttac

SEQ ID NO: 73 - DNA, 10 bp, Homo sapiens
tcccacaagg

SEQ ID NO: 74 - DNA, 10 bp, Homo sapiens
taaatcccca

SEQ ID NO: 75 - DNA, 10 bp, Homo sapiens
cccgcccccg

SEQ ID NO: 76 - DNA, 10 bp, Homo sapiens
cccgaggcag

SEQ ID NO: 77 - DNA, 10 bp, Homo sapiens
ctacgtgatg

SEQ ID NO: 78 - DNA, 10 bp, Homo sapiens
atgggtttgc

SEQ ID NO: 79 - DNA, 10 bp, Homo sapiens
ggcattgtct

SEQ ID NO: 80 - DNA, 10 bp, Homo sapiens
gtgctaagcg

SEQ ID NO: 81 - DNA, 10 bp, Homo sapiens
accgtttgca

SEQ ID NO: 82 - DNA, 10 bp, Homo sapiens
cagcgctgca

SEQ ID NO: 83 - DNA, 10 bp, Homo sapiens
gaagacactt

SEQ ID NO: 84 - DNA, 10 bp, Homo sapiens
cgctgggcgt

SEQ ID NO: 85 - DNA, 10 bp, Homo sapiens
cacccctgat

SEQ ID NO: 86 - DNA, 10 bp, Homo sapiens
gcccccctgc

SEQ ID NO: 87 - DNA, 10 bp, Homo sapiens
ccccctgccc

SEQ ID NO: 88 - DNA, 10 bp, Homo sapiens
agcataaaaa

SEQ ID NO: 89 - DNA, 10 bp, Homo sapiens
gggctggacg

SEQ ID NO: 90 - DNA, 10 bp, Homo sapiens
ctgccaactt

SEQ ID NO: 91 - DNA, 10 bp, Homo sapiens
aagtggatag

SEQ ID NO: 92 - DNA, 10 bp, Homo sapiens
cgtactgagc

SEQ ID NO: 93 - DNA, 10 bp, Homo sapiens
ccgcttactc

SEQ ID NO: 94 - DNA, 10 bp, Homo sapiens
ggggcttctg

SEQ ID NO: 95 - DNA, 10 bp, Homo sapiens
cccgtccgga

SEQ ID NO: 96 - DNA, 10 bp, Homo sapiens
agttccacca

SEQ ID NO: 97 - DNA, 10 bp, Homo sapiens
ggcctccagc

SEQ ID NO: 98 - DNA, 10 bp, Homo sapiens
ggaggctgag

SEQ ID NO: 99 - DNA, 10 bp, Homo sapiens
cagaggcgtc

SEQ ID NO: 100 - DNA, 10 bp, Homo sapiens
gaccagcctt

SEQ ID NO: 101 - DNA, 10 bp, Homo sapiens
gaggatggtg

SEQ ID NO: 102 - DNA, 10 bp, Homo sapiens
tcgtcgcaga

SEQ ID NO: 103 - DNA, 10 bp, Homo sapiens
ggggctgccc

SEQ ID NO: 104 - DNA, 10 bp, Homo sapiens
ctgtacatac

SEQ ID NO: 105 - DNA, 10 bp, Homo sapiens
gcgacgaggc

SEQ ID NO: 106 - DNA, 10 bp, Homo sapiens
gccaagtgaa

SEQ ID NO: 107 - DNA, 10 bp, Homo sapiens
aagataaact

SEQ ID NO: 108 - DNA, 10 bp, Homo sapiens
gagagtgtac

SEQ ID NO: 109 - DNA, 10 bp, Homo sapiens
ccactgcact

SEQ ID NO: 110 - DNA, 10 bp, Homo sapiens
ccaccctcac

SEQ ID NO: 111 - DNA, 10 bp, Homo sapiens
cagaccattg

SEQ ID NO: 112 - DNA, 10 bp, Homo sapiens
gggagctgcg

SEQ ID NO: 113 - DNA, 10 bp, Homo sapiens
gggatttctg

SEQ ID NO: 114 - DNA, 10 bp, Homo sapiens
cttccagcta

SEQ ID NO: 115 - DNA, 10 bp, Homo sapiens
cagaaacaga

SEQ ID NO: 116 - DNA, 10 bp, Homo sapiens
tctgtgctca

SEQ ID NO: 117 - DNA, 10 bp, Homo sapiens
tgcaataggt

SEQ ID NO: 118 - DNA, 10 bp, Homo sapiens
atggccaact

SEQ ID NO: 119 - DNA, 10 bp, Homo sapiens
tcacacagtg

SEQ ID NO: 120 - DNA, 10 bp, Homo sapiens
ggcttaggat

SEQ ID NO: 121 - DNA, 10 bp, Homo sapiens
gggaggggtg

SEQ ID NO: 122 - DNA, 10 bp, Homo sapiens
gaagtagaag

SEQ ID NO: 123 - DNA, 10 bp, Homo sapiens
caccctgtac

SEQ ID NO: 124 - DNA, 10 bp, Homo sapiens
atgtttacaa

SEQ ID NO: 125 - DNA, 10 bp, Homo sapiens
caaactggtc

SEQ ID NO: 126 - DNA, 10 bp, Homo sapiens
gtaatgacag

SEQ ID NO: 127 - DNA, 10 bp, Homo sapiens
acctgccgac

SEQ ID NO: 128 - DNA, 10 bp, Homo sapiens
tgatgcgcgc

SEQ ID NO: 129 - DNA, 10 bp, Homo sapiens
tggccccagg

SEQ ID NO: 130 - DNA, 10 bp, Homo sapiens
gcctgctggg

SEQ ID NO: 131 - DNA, 10 bp, Homo sapiens
tgcctgtggt

SEQ ID NO: 132 - DNA, 10 bp, Homo sapiens
gagggtatac

SEQ ID NO: 133 - DNA, 10 bp, Homo sapiens
ggagccagct

SEQ ID NO: 134 - DNA, 10 bp, Homo sapiens
gagcctcagg

SEQ ID NO: 135 - DNA, 10 bp, Homo sapiens
tacttcacat

SEQ ID NO: 136 - DNA, 10 bp, Homo sapiens
taatcccagc

SEQ ID NO: 137 - DNA, 10 bp, Homo sapiens
caccttccag

SEQ ID NO: 138 - DNA, 10 bp, Homo sapiens
gagtctgttc

SEQ ID NO: 139 - DNA, 10 bp, Homo sapiens
ggattttggt

SEQ ID NO: 140 - DNA, 10 bp, Homo sapiens
tgcctgtagt

SEQ ID NO: 141 - DNA, 10 bp, Homo sapiens
ttacaaacag

SEQ ID NO: 142 - DNA, 10 bp, Homo sapiens
tcttctttca

SEQ ID NO: 143 - DNA, 10 bp, Homo sapiens
agcacatttg

SEQ ID NO: 144 - DNA, 10 bp, Homo sapiens
cagggctcgc

SEQ ID NO: 145 - DNA, 10 bp, Homo sapiens
gctggtccca

SEQ ID NO: 146 - DNA, 10 bp, Homo sapiens
tccacgccct

SEQ ID NO: 147 - DNA, 10 bp, Homo sapiens
ttgcaatagc

SEQ ID NO: 148 - DNA, 10 bp, Homo sapiens
agggcttcca

SEQ ID NO: 149 - DNA, 10 bp, Homo sapiens
ctgggttaat

SEQ ID NO: 150 - DNA, 10 bp, Homo sapiens
aacctgggag

SEQ ID NO: 151 - DNA, 10 bp, Homo sapiens
ggcaacgtgg

SEQ ID NO: 152 - DNA, 10 bp, Homo sapiens
ggatgcgcag

SEQ ID NO: 153 - DNA, 10 bp, Homo sapiens
cacctgtagt

SEQ ID NO: 154 - DNA, 10 bp, Homo sapiens
gtggtgggcg

SEQ ID NO: 155 - DNA, 10 bp, Homo sapiens
gcagggtggg

SEQ ID NO: 156 - DNA, 10 bp, Homo sapiens
caagcatccc

SEQ ID NO: 157 - DNA, 10 bp, Homo sapiens
tgggggccga

SEQ ID NO: 158 - DNA, 10 bp, Homo sapiens
tcagtgtatt

SEQ ID NO: 159 - DNA, 10 bp, Homo sapiens
ggcaagcccc

SEQ ID NO: 160 - DNA, 10 bp, Homo sapiens
cctagctgga

SEQ ID NO: 161 - DNA, 10 bp, Homo sapiens
gcaaaaccct

SEQ ID NO: 162 - DNA, 10 bp, Homo sapiens
gctggttcct

SEQ ID NO: 163 - DNA, 10 bp, Homo sapiens
gcacctcagc

SEQ ID NO: 164 - DNA, 10 bp, Homo sapiens
accagctgtc

SEQ ID NO: 165 - DNA, 10 bp, Homo sapiens
tttgaatcag

SEQ ID NO: 166 - DNA, 10 bp, Homo sapiens
agactagggg

SEQ ID NO: 167 - DNA, 10 bp, Homo sapiens
agctcagtga

SEQ ID NO: 168 - DNA, 10 bp, Homo sapiens
ggccaacatt

SEQ ID NO: 169 - DNA, 10 bp, Homo sapiens
tttgtgggca

SEQ ID NO: 170 - DNA, 10 bp, Homo sapiens
attgtagaca

SEQ ID NO: 171 - DNA, 10 bp, Homo sapiens
ccctaggttg

SEQ ID NO: 172 - DNA, 10 bp, Homo sapiens
aaatcaccaa

SEQ ID NO: 173 - DNA, 10 bp, Homo sapiens
ggctgcagtc

SEQ ID NO: 174 - DNA, 10 bp, Homo sapiens
gtggcaggcg

SEQ ID NO: 175 - DNA, 10 bp, Homo sapiens
taaaggcaca

SEQ ID NO: 176 - DNA, 10 bp, Homo sapiens
ggctcctggc

SEQ ID NO: 177 - DNA, 10 bp, Homo sapiens
atattaggaa

SEQ ID NO: 178 - DNA, 10 bp, Homo sapiens
gcttcagtgg

SEQ ID NO: 179 - DNA, 10 bp, Homo sapiens
tgattaaaac

SEQ ID NO: 180 - DNA, 10 bp, Homo sapiens
agccaccacg

SEQ ID NO: 181 - DNA, 10 bp, Homo sapiens
ggcggctgca

SEQ ID NO: 182 - DNA, 10 bp, Homo sapiens
tgtttggggg

SEQ ID NO: 183 - DNA, 10 bp, Homo sapiens
cctgcctcgt

SEQ ID NO: 184 - DNA, 10 bp, Homo sapiens
aggcctgggc

SEQ ID NO: 185 - DNA, 10 bp, Homo sapiens
caaaactgtt

SEQ ID NO: 186 - DNA, 10 bp, Homo sapiens
gagaggacat

SEQ ID NO: 187 - DNA, 10 bp, Homo sapiens
gagttaggca

SEQ ID NO: 188 - DNA, 10 bp, Homo sapiens
ccgtagtgcc

SEQ ID NO: 189 - DNA, 10 bp, Homo sapiens
cataaacggg

SEQ ID NO: 190 - DNA, 10 bp, Homo sapiens
tccctggcag

SEQ ID NO: 191 - DNA, 10 bp, Homo sapiens
gaggccatcc

SEQ ID NO: 192 - DNA, 10 bp, Homo sapiens
ttgcctggga

SEQ ID NO: 193 - DNA, 10 bp, Homo sapiens
ctgtcagcgg

SEQ ID NO: 194 - DNA, 10 bp, Homo sapiens
aacgcggcca

SEQ ID NO: 195 - DNA, 10 bp, Homo sapiens
ggtttggctt

SEQ ID NO: 196 - DNA, 10 bp, Homo sapiens
gatttttgtg

SEQ ID NO: 197 - DNA, 10 bp, Homo sapiens
ggctgccctg

SEQ ID NO: 198 - DNA, 10 bp, Homo sapiens
atggcaacag

SEQ ID NO: 199 - DNA, 10 bp, Homo sapiens
cgctgtgggg

SEQ ID NO: 200 - DNA, 10 bp, Homo sapiens
ggcagccaga

SEQ ID NO: 201 - DNA, 10 bp, Homo sapiens
agagcaaacc

SEQ ID NO: 202 - DNA, 10 bp, Homo sapiens
tttccctcaa

SEQ ID NO: 203 - DNA, 10 bp, Homo sapiens
tccccgtggc

SEQ ID NO: 204 - DNA, 10 bp, Homo sapiens
ttctcccaaa

SEQ ID NO: 205 - DNA, 10 bp, Homo sapiens
ggctgggggc

SEQ ID NO: 206 - DNA, 10 bp, Homo sapiens
ccctaccctg

SEQ ID NO: 207 - DNA, 10 bp, Homo sapiens
taggaccctg

SEQ ID NO: 208 - DNA, 10 bp, Homo sapiens
gtttttgctt

SEQ ID NO: 209 - DNA, 10 bp, Homo sapiens
cttgattccc

SEQ ID NO: 210 - DNA, 10 bp, Homo sapiens
gcttggctcc

SEQ ID NO: 211 - DNA, 10 bp, Homo sapiens
ggtggcactc

SEQ ID NO: 212 - DNA, 10 bp, Homo sapiens
acctgtgacc

SEQ ID NO: 213 - DNA, 10 bp, Homo sapiens
actgaggaaa

SEQ ID NO: 214 - DNA, 10 bp, Homo sapiens
tgcagcgcct

SEQ ID NO: 215 - DNA, 10 bp, Homo sapiens
ctggggggaa

SEQ ID NO: 216 - DNA, 10 bp, Homo sapiens
gtgctattct

SEQ ID NO: 217 - DNA, 10 bp, Homo sapiens
ggagggggct

SEQ ID NO: 218 - DNA, 10 bp, Homo sapiens
gtgcctgaga

SEQ ID NO: 219 - DNA, 10 bp, Homo sapiens
tcacagggtc

SEQ ID NO: 220 - DNA, 10 bp, Homo sapiens
gggctccctg

SEQ ID NO: 221 - DNA, 10 bp, Homo sapiens
gccccaggta

SEQ ID NO: 222 - DNA, 10 bp, Homo sapiens
gaaagtggct

SEQ ID NO: 223 - DNA, 10 bp, Homo sapiens
tccctggctg

SEQ ID NO: 224 - DNA, 10 bp, Homo sapiens
acagagcaca

SEQ ID NO: 225 - DNA, 10 bp, Homo sapiens
ctttgcactc

SEQ ID NO: 226 - DNA, 10 bp, Homo sapiens
atgctccctg

SEQ ID NO: 227 - DNA, 10 bp, Homo sapiens
ccgtccaagg

SEQ ID NO: 228 - DNA, 10 bp, Homo sapiens
gggccccctg

SEQ ID NO: 229 - DNA, 10 bp, Homo sapiens
cttatgctgc

SEQ ID NO: 230 - DNA, 10 bp, Homo sapiens
ggttattttg

SEQ ID NO: 231 - DNA, 10 bp, Homo sapiens
gcctgtccct

SEQ ID NO: 232 - DNA, 10 bp, Homo sapiens
aagatgaggg

SEQ ID NO: 233 - DNA, 10 bp, Homo sapiens
ccaacaagaa

SEQ ID NO: 234 - DNA, 10 bp, Homo sapiens
aaggatgcgg

SEQ ID NO: 235 - DNA, 10 bp, Homo sapiens
tgtcatcaca

SEQ ID NO: 236 - DNA, 10 bp, Homo sapiens
caggcttttt

SEQ ID NO: 237 - DNA, 10 bp, Homo sapiens
tcaagttcac

SEQ ID NO: 238 - DNA, 10 bp, Homo sapiens
tccctgggca

SEQ ID NO: 239 - DNA, 10 bp, Homo sapiens
caggagttca

SEQ ID NO: 240 - DNA, 10 bp, Homo sapiens
caggtggttc

SEQ ID NO: 241 - DNA, 10 bp, Homo sapiens
gcccacatcc

SEQ ID NO: 242 - DNA, 10 bp, Homo sapiens
gctggggtgg

SEQ ID NO: 243 - DNA, 10 bp, Homo sapiens
gacctcctgc

SEQ ID NO: 244 - DNA, 10 bp, Homo sapiens
agtgaataaa

SEQ ID NO: 245 - DNA, 10 bp, Homo sapiens
aaggttcttc

SEQ ID NO: 246 - DNA, 10 bp, Homo sapiens
agcctggact

SEQ ID NO: 247 - DNA, 10 bp, Homo sapiens
caacccagat

SEQ ID NO: 248 - DNA, 10 bp, Homo sapiens
tgcttctgcc

SEQ ID NO: 249 - DNA, 10 bp, Homo sapiens
caggtgacaa

SEQ ID NO: 250 - DNA, 10 bp, Homo sapiens
ggccgggggc

SEQ ID NO: 251 - DNA, 10 bp, Homo sapiens
gtgcgctagg

SEQ ID NO: 252 - DNA, 10 bp, Homo sapiens
aggctgtcca

SEQ ID NO: 253 - DNA, 10 bp, Homo sapiens
tgttatgtcc

SEQ ID NO: 254 - DNA, 10 bp, Homo sapiens
tttcccaaac

SEQ ID NO: 255 - DNA, 10 bp, Homo sapiens
ggggatgggg

SEQ ID NO: 256 - DNA, 17 bp, Homo sapiens
aaaccattctcctccgc

SEQ ID NO: 257 - DNA, 17 bp, Homo sapiens
aaggcagggagggaggg

SEQ ID NO: 258 - DNA, 17 bp, Homo sapiens
acacagcaagacgagaa

SEQ ID NO: 259 - DNA, 17 bp, Homo sapiens
agctggagtcctaggca

SEQ ID NO: 260 - DNA, 17 bp, Homo sapiens
agctggcaccagagccc

SEQ ID NO: 261 - DNA, 17 bp, Homo sapiens
ataaatgaggtaaggtc

SEQ ID NO: 262 - DNA, 17 bp, Homo sapiens
caagcacccccgttcca

SEQ ID NO: 263 - DNA, 17 bp, Homo sapiens
cactacccaccagacgc

SEQ ID NO: 264 - DNA, 17 bp, Homo sapiens
cactactcaccaggcgc

SEQ ID NO: 265 - DNA, 17 bp, Homo sapiens
cccacctccagtccagc

SEQ ID NO: 266 - DNA, 17 bp, Homo sapiens
cccgcctcttcacgggc

SEQ ID NO: 267 - DNA, 17 bp, Homo sapiens
cctcagatgtttgaaaa

SEQ ID NO: 268 - DNA, 17 bp, Homo sapiens
cgctactcaccagacgc

SEQ ID NO: 269 - DNA, 17 bp, Homo sapiens
ctaagacctcaccagtc

SEQ ID NO: 270 - DNA, 17 bp, Homo sapiens
ctaagacttcaccggtc

SEQ ID NO: 271 - DNA, 17 bp, Homo sapiens
gagtgggtgcagcctcc

SEQ ID NO: 272 - DNA, 17 bp, Homo sapiens
gggacagctgtctgtgg

SEQ ID NO: 273 - DNA, 17 bp, Homo sapiens
gggttggcttgaaacca

SEQ ID NO: 274 - DNA, 17 bp, Homo sapiens
gtaagtgtactggaagt

SEQ ID NO: 275 - DNA, 17 bp, Homo sapiens
gtaagtgtactggtaag

SEQ ID NO: 276 - DNA, 17 bp, Homo sapiens
gtaggggtaaaaggagg

SEQ ID NO: 277 - DNA, 17 bp, Homo sapiens
taaccactgcactttcc

SEQ ID NO: 278 - DNA, 17 bp, Homo sapiens
tactgctcggaggtcgg

SEQ ID NO: 279 - DNA, 17 bp, Homo sapiens
tcaggctgaagtcaggc

SEQ ID NO: 280 - DNA, 17 bp, Homo sapiens
tccatacacctatcccc

SEQ ID NO: 281 - DNA, 17 bp, Homo sapiens
tccttttaaaacaaaac

SEQ ID NO: 282 - DNA, 17 bp, Homo sapiens
tgattaaggtcggcgct

SEQ ID NO: 283 - DNA, 17 bp, Homo sapiens
tggtatcacacaagggg

SEQ ID NO: 284 - DNA, 17 bp, Homo sapiens
tggtgtatgcatcgggg

SEQ ID NO: 285 - DNA, 17 bp, Homo sapiens
tgtcactgggcaggcgg

SEQ ID NO: 286 - DNA, 17 bp, Homo sapiens
tgtgggaggctgatggg

SEQ ID NO: 287 - DNA, 17 bp, Homo sapiens
tttaacggccgcggtac

SEQ ID NO: 288 - DNA, 17 bp, Homo sapiens
gctctctatgctgacgt

SEQ ID NO: 289 - DNA, 17 bp, Homo sapiens
agaatgaaactgccggg

SEQ ID NO: 290 - DNA, 17 bp, Homo sapiens
aagtggaataaactgcc

SEQ ID NO: 291 - DNA, 17 bp, Homo sapiens
gatgacgactcggggct

SEQ ID NO: 292 - DNA, 17 bp, Homo sapiens
ccctttcacacacactt

SEQ ID NO: 293 - DNA, 17 bp, Homo sapiens
tcctggggcaggggcgg

SEQ ID NO: 294 - DNA, 17 bp, Homo sapiens
tctattgatgtgtatgc

SEQ ID NO: 295 - DNA, 17 bp, Homo sapiens
ggggctgtatttaagga

SEQ ID NO: 296 - DNA, 17 bp, Homo sapiens
cccaggacaccagctgg

SEQ ID NO: 297 - DNA, 17 bp, Homo sapiens
ggagctgctgcttgtgg

SEQ ID NO: 298 - DNA, 17 bp, Homo sapiens
tggacagcagggacctg

SEQ ID NO: 299 - DNA, 17 bp, Homo sapiens
tctgggaacagggacgg

SEQ ID NO: 300 - DNA, 17 bp, Homo sapiens
cctgtgtatgtgtgtaa

SEQ ID NO: 301 - DNA, 17 bp, Homo sapiens
ggcaagaagaagatcgc

SEQ ID NO: 302 - DNA, 17 bp, Homo sapiens
aaatgcttggaggtgaa

SEQ ID NO: 303 - DNA, 17 bp, Homo sapiens
ctaaaaaccttatgaca

SEQ ID NO: 304 - DNA, 17 bp, Homo sapiens
gagcattgcaccacccg

SEQ ID NO: 305 - DNA, 17 bp, Homo sapiens
ggtggacacggatctgc

SEQ ID NO: 306 - DNA, 17 bp, Homo sapiens
gctcctgagccccggcc

SEQ ID NO: 307 - DNA, 17 bp, Homo sapiens
aagaagtggagattgtc

SEQ ID NO: 308 - DNA, 17 bp, Homo sapiens
tgggaagtgggctcctt

SEQ ID NO: 309 - DNA, 17 bp, Homo sapiens
actcgctctgtggaggt

SEQ ID NO: 310 - DNA, 17 bp, Homo sapiens
tttcaggggagggggaa

SEQ ID NO: 311 - DNA, 17 bp, Homo sapiens
acaacgtccagctggtg

SEQ ID NO: 312 - DNA, 17 bp, Homo sapiens
gtctcagtgctgaggcg

SEQ ID NO: 313 - DNA, 17 bp, Homo sapiens
ccccctgcccctctgcc

SEQ ID NO: 314 - DNA, 17 bp, Homo sapiens
agaaaccacggaaatgg

SEQ ID NO: 315 - DNA, 17 bp, Homo sapiens
gaccgcaggagggcaga

SEQ ID NO: 316 - DNA, 17 bp, Homo sapiens
gtgctacttcttcttct

SEQ ID NO: 317 - DNA, 17 bp, Homo sapiens
gataactacattacctg

SEQ ID NO: 318 - DNA, 17 bp, Homo sapiens
tggctgtgactgtgact

SEQ ID NO: 319 - DNA, 17 bp, Homo sapiens
gagtgagacccaggagc

SEQ ID NO: 320 - DNA, 17 bp, Homo sapiens
gagtggctacccgccgc

SEQ ID NO: 321 - DNA, 17 bp, Homo sapiens
gactcagggatttgttg

SEQ ID NO: 322 - DNA, 17 bp, Homo sapiens
gttatatgcccgggaga

SEQ ID NO: 323 - DNA, 17 bp, Homo sapiens
gaggcgctgctgccacc

SEQ ID NO: 324 - DNA, 17 bp, Homo sapiens
gagctctgagatcaccc

SEQ ID NO: 325 - DNA, 17 bp, Homo sapiens
gccagccagtggcaagc

SEQ ID NO: 326 - DNA, 17 bp, Homo sapiens
atggcaacagatctgga

SEQ ID NO: 327 - DNA, 17 bp, Homo sapiens
aaggagttacactagtc

SEQ ID NO: 328 - DNA, 17 bp, Homo sapiens
tcccacaaggctgcttg

SEQ ID NO: 329 - DNA, 17 bp, Homo sapiens
taaatccccactgggac

SEQ ID NO: 330 - DNA, 17 bp, Homo sapiens
cccgcccccgccttccc

SEQ ID NO: 331 - DNA, 17 bp, Homo sapiens
cccgaggcagagtcggg

SEQ ID NO: 332 - DNA, 17 bp, Homo sapiens
ctacgtgatgaagatgg

SEQ ID NO: 333 - DNA, 17 bp, Homo sapiens
atgggtttgcattttag

SEQ ID NO: 334 - DNA, 17 bp, Homo sapiens
ggcattgtctctgtttc

SEQ ID NO: 335 - DNA, 17 bp, Homo sapiens
gtgctaagcgggcccgg

SEQ ID NO: 336 - DNA, 17 bp, Homo sapiens
accgtttgcattcgaaa

SEQ ID NO: 337 - DNA, 17 bp, Homo sapiens
cagcgctgcattgactc

SEQ ID NO: 338 - DNA, 17 bp, Homo sapiens
gaagacacttggtttga

SEQ ID NO: 339 - DNA, 17 bp, Homo sapiens
cgctgggcgtctgggac

SEQ ID NO: 340 - DNA, 17 bp, Homo sapiens
cacccctgatgttcgcc

SEQ ID NO: 341 - DNA, 17 bp, Homo sapiens
gcccccctgccccgtgc

SEQ ID NO: 342 - DNA, 17 bp, Homo sapiens
ccccctgccctcgcctg

SEQ ID NO: 343 - DNA, 17 bp, Homo sapiens
agcataaaaatgcgtgc

SEQ ID NO: 344 - DNA, 17 bp, Homo sapiens
gggctggacggctgcgt

SEQ ID NO: 345 - DNA, 17 bp, Homo sapiens
ctgccaacttctaaccg

SEQ ID NO: 346 - DNA, 17 bp, Homo sapiens
aagtggatagatacttc

SEQ ID NO: 347 - DNA, 17 bp, Homo sapiens
cgtactgagcgctttgg

SEQ ID NO: 348 - DNA, 17 bp, Homo sapiens
ccgcttactctgttggg

SEQ ID NO: 349 - DNA, 17 bp, Homo sapiens
ggggcttctgtagcccc

SEQ ID NO: 350 - DNA, 17 bp, Homo sapiens
cccgtccggaacgtcta

SEQ ID NO: 351 - DNA, 17 bp, Homo sapiens
agttccaccagaaagcc

SEQ ID NO: 352 - DNA, 17 bp, Homo sapiens
ggcctccagccacccac

SEQ ID NO: 353 - DNA, 17 bp, Homo sapiens
ggaggctgaggtgggag

SEQ ID NO: 354 - DNA, 17 bp, Homo sapiens
cagaggcgtccgcaggt

SEQ ID NO: 355 - DNA, 17 bp, Homo sapiens
gaccagccttcagatgg

SEQ ID NO: 356 - DNA, 17 bp, Homo sapiens
gaggatggtgtcctgag

SEQ ID NO: 357 - DNA, 17 bp, Homo sapiens
tcgtcgcagaaggcgct

SEQ ID NO: 358 - DNA, 17 bp, Homo sapiens
ggggctgcccagctgga

SEQ ID NO: 359 - DNA, 17 bp, Homo sapiens
ctgtacatactttttgg

SEQ ID NO: 360 - DNA, 17 bp, Homo sapiens
gcgacgaggcgcgctgg

SEQ ID NO: 361 - DNA, 17 bp, Homo sapiens
gccaagtgaactgtggc

SEQ ID NO: 362 - DNA, 17 bp, Homo sapiens
aagataaactctgggcc

SEQ ID NO: 363 - DNA, 17 bp, Homo sapiens
gagagtgtactggcact

SEQ ID NO: 364 - DNA, 17 bp, Homo sapiens
ccactgcactccggcct

SEQ ID NO: 365 - DNA, 17 bp, Homo sapiens
ccaccctcacacacaca

SEQ ID NO: 366 - DNA, 17 bp, Homo sapiens
cagaccattgtttgatc

SEQ ID NO: 367 - DNA, 17 bp, Homo sapiens
gggagctgcgccaacgg

SEQ ID NO: 368 - DNA, 17 bp, Homo sapiens
gggatttctgtgtctgc

SEQ ID NO: 369 - DNA, 17 bp, Homo sapiens
cttccagctaacaggtc

SEQ ID NO: 370 - DNA, 17 bp, Homo sapiens
cagaaacagactggggg

SEQ ID NO: 371 - DNA, 17 bp, Homo sapiens
tctgtgctcaggaagag

SEQ ID NO: 372 - DNA, 17 bp, Homo sapiens
tgcaataggtgagagaa

SEQ ID NO: 373 - DNA, 17 bp, Homo sapiens
atggccaacttccacct

SEQ ID NO: 374 - DNA, 17 bp, Homo sapiens
tcacacagtgcctgtcg

SEQ ID NO: 375 - DNA, 17 bp, Homo sapiens
ggcttaggatgtgaatg

SEQ ID NO: 376 - DNA, 17 bp, Homo sapiens
gggaggggtgggggtgg

SEQ ID NO: 377 - DNA, 17 bp, Homo sapiens
gaagtagaaggtaagga

SEQ ID NO: 378 - DNA, 17 bp, Homo sapiens
caccctgtacagttgcc

SEQ ID NO: 379 - DNA, 17 bp, Homo sapiens
atgtttacaagatggcg

SEQ ID NO: 380 - DNA, 17 bp, Homo sapiens
caaactggtctaggtca

SEQ ID NO: 381 - DNA, 17 bp, Homo sapiens
gtaatgacagatgcaag

SEQ ID NO: 382 - DNA, 17 bp, Homo sapiens
acctgccgacagtgttg

SEQ ID NO: 383 - DNA, 17 bp, Homo sapiens
tgatgcgcgctttgttg

SEQ ID NO: 384 - DNA, 17 bp, Homo sapiens
tggccccaggtgccacc

SEQ ID NO: 385 - DNA, 17 bp, Homo sapiens
gcctgctgggcttggct

SEQ ID NO: 386 - DNA, 17 bp, Homo sapiens
tgcctgtggtcccagct

SEQ ID NO: 387 - DNA, 17 bp, Homo sapiens
gagggtatactgagggg

SEQ ID NO: 388 - DNA, 17 bp, Homo sapiens
ggagccagctgacctgc

SEQ ID NO: 389 - DNA, 17 bp, Homo sapiens
gagcctcaggtgctccc

SEQ ID NO: 390 - DNA, 17 bp, Homo sapiens
tacttcacatacagtgc

SEQ ID NO: 391 - DNA, 17 bp, Homo sapiens
taatcccagcactttgg

SEQ ID NO: 392 - DNA, 17 bp, Homo sapiens
caccttccagcccgggg

SEQ ID NO: 393 - DNA, 17 bp, Homo sapiens
gagtctgttcgtgactc

SEQ ID NO: 394 - DNA, 17 bp, Homo sapiens
ggattttggtctctgtc

SEQ ID NO: 395 - DNA, 17 bp, Homo sapiens
tgcctgtagtcctagtt

SEQ ID NO: 396 - DNA, 17 bp, Homo sapiens
ttacaaacagaaaagct

SEQ ID NO: 397 - DNA, 17 bp, Homo sapiens
tcttctttcagaatggg

SEQ ID NO: 398 - DNA, 17 bp, Homo sapiens
agcacatttgatatagc

SEQ ID NO: 399 - DNA, 17 bp, Homo sapiens
cagggctcgcgtgcggg

SEQ ID NO: 400 - DNA, 17 bp, Homo sapiens
gctggtcccagggccag

SEQ ID NO: 401 - DNA, 17 bp, Homo sapiens
tccacgcccttcctggc

SEQ ID NO: 402 - DNA, 17 bp, Homo sapiens
ttgcaatagcaaaaccc

SEQ ID NO: 403 - DNA, 17 bp, Homo sapiens
agggcttccaatgtgct

SEQ ID NO: 404 - DNA, 17 bp, Homo sapiens
ctgggttaataaattgc

SEQ ID NO: 405 - DNA, 17 bp, Homo sapiens
aacctgggaggtggagg

SEQ ID NO: 406 - DNA, 17 bp, Homo sapiens
ggcaacgtggtagaggc

SEQ ID NO: 407 - DNA, 17 bp, Homo sapiens
ggatgcgcaggggaggc

SEQ ID NO: 408 - DNA, 17 bp, Homo sapiens
cacctgtagtcctagct

SEQ ID NO: 409 - DNA, 17 bp, Homo sapiens
gtggtgggcgcctgtag

SEQ ID NO: 410 - DNA, 17 bp, Homo sapiens
gcagggtggggaggggt

SEQ ID NO: 411 - DNA, 17 bp, Homo sapiens
caagcatccccgttcca

SEQ ID NO: 412 - DNA, 17 bp, Homo sapiens
tgggggccgatgggcag

SEQ ID NO: 413 - DNA, 17 bp, Homo sapiens
tcagtgtattaaaaccc

SEQ ID NO: 414 - DNA, 17 bp, Homo sapiens
ggcaagccccagcgcct

SEQ ID NO: 415 - DNA, 17 bp, Homo sapiens
cctagctggattgcaga

SEQ ID NO: 416 - DNA, 17 bp, Homo sapiens
gcaaaaccctgctctcc

SEQ ID NO: 417 - DNA, 17 bp, Homo sapiens
gctggttcctgagtggc

SEQ ID NO: 418 - DNA, 17 bp, Homo sapiens
gcacctcagccaggggt

SEQ ID NO: 419 - DNA, 17 bp, Homo sapiens
accagctgtccaggggc

SEQ ID NO: 420 - DNA, 17 bp, Homo sapiens
tttgaatcagtgctaga

SEQ ID NO: 421 - DNA, 17 bp, Homo sapiens
agactaggggccggagc

SEQ ID NO: 422 - DNA, 17 bp, Homo sapiens
agctcagtgagaagggc

SEQ ID NO: 423 - DNA, 17 bp, Homo sapiens
ggccaacatttggtcca

SEQ ID NO: 424 - DNA, 17 bp, Homo sapiens
tttgtgggcagtcaggc

SEQ ID NO: 425 - DNA, 17 bp, Homo sapiens
attgtagacaatgaggg

SEQ ID NO: 426 - DNA, 17 bp, Homo sapiens
ccctaggttgggcccct

SEQ ID NO: 427 - DNA, 17 bp, Homo sapiens
aaatcaccaatcaaggc

SEQ ID NO: 428 - DNA, 17 bp, Homo sapiens
ggctgcagtcttcttcc

SEQ ID NO: 429 - DNA, 17 bp, Homo sapiens
gtggcaggcgcctgtag

SEQ ID NO: 430 - DNA, 17 bp, Homo sapiens
taaaggcacagtggctc

SEQ ID NO: 431 - DNA, 17 bp, Homo sapiens
ggctcctggctctggac

SEQ ID NO: 432 - DNA, 17 bp, Homo sapiens
atattaggaagtcgggg

SEQ ID NO: 433 - DNA, 17 bp, Homo sapiens
gcttcagtgggggagag

SEQ ID NO: 434 - DNA, 17 bp, Homo sapiens
tgattaaaacaagttgc

SEQ ID NO: 435 - DNA, 17 bp, Homo sapiens
agccaccacgcctggtc

SEQ ID NO: 436 - DNA, 17 bp, Homo sapiens
ggcggctgcagagcctg

SEQ ID NO: 437 - DNA, 17 bp, Homo sapiens
tgtttgggggcttttag

SEQ ID NO: 438 - DNA, 17 bp, Homo sapiens
cctgcctcgtagtgaag

SEQ ID NO: 439 - DNA, 17 bp, Homo sapiens
aggcctgggcctctgcg

SEQ ID NO: 440 - DNA, 17 bp, Homo sapiens
caaaactgtttgttggc

SEQ ID NO: 441 - DNA, 17 bp, Homo sapiens
gagaggacattggaggg

SEQ ID NO: 442 - DNA, 17 bp, Homo sapiens
gagttaggcacttcctg

SEQ ID NO: 443 - DNA, 17 bp, Homo sapiens
ccgtagtgcctttatgg

SEQ ID NO: 444 - DNA, 17 bp, Homo sapiens
cataaacgggcacaccc

SEQ ID NO: 445 - DNA, 17 bp, Homo sapiens
tccctggcagagggctt

SEQ ID NO: 446 - DNA, 17 bp, Homo sapiens
gaggccatccccaaccc

SEQ ID NO: 447 - DNA, 17 bp, Homo sapiens
ttgcctgggatgctggt

SEQ ID NO: 448 - DNA, 17 bp, Homo sapiens
ctgtcagcggctgcccc

SEQ ID NO: 449 - DNA, 17 bp, Homo sapiens
aacgcggccaatgtggg

SEQ ID NO: 450 - DNA, 17 bp, Homo sapiens
ggtttggcttaggctgg

SEQ ID NO: 451 - DNA, 17 bp, Homo sapiens
gatttttgtggtgtggg

SEQ ID NO: 452 - DNA, 17 bp, Homo sapiens
ggctgccctgggcagcc

SEQ ID NO: 453 - DNA, 17 bp, Homo sapiens
atggcaacagaaaccaa

SEQ ID NO: 454 - DNA, 17 bp, Homo sapiens
cgctgtggggtgcagac

SEQ ID NO: 455 - DNA, 17 bp, Homo sapiens
ggcagccagagctccaa

SEQ ID NO: 456 - DNA, 17 bp, Homo sapiens
agagcaaaccgtagtcc

SEQ ID NO: 457 - DNA, 17 bp, Homo sapiens
tttccctcaaagactct

SEQ ID NO: 458 - DNA, 17 bp, Homo sapiens
tccccgtggctgtgggg

SEQ ID NO: 459 - DNA, 17 bp, Homo sapiens
ttctcccaaataccgtt

SEQ ID NO: 460 - DNA, 17 bp, Homo sapiens
ggctgggggccagggct

SEQ ID NO: 461 - DNA, 17 bp, Homo sapiens
ccctaccctgttacctt

SEQ ID NO: 462 - DNA, 17 bp, Homo sapiens
taggaccctgcaggggg

SEQ ID NO: 463 - DNA, 17 bp, Homo sapiens
gtttttgcttcagcggc

SEQ ID NO: 464 - DNA, 17 bp, Homo sapiens
cttgattcccacgctac

SEQ ID NO: 465 - DNA, 17 bp, Homo sapiens
gcttggctcccaaaggg

SEQ ID NO: 466 - DNA, 17 bp, Homo sapiens
ggtggcactcagtctct

SEQ ID NO: 467 - DNA, 17 bp, Homo sapiens
acctgtgaccagcactg

SEQ ID NO: 468 - DNA, 17 bp, Homo sapiens
actgaggaaaggagctc

SEQ ID NO: 469 - DNA, 17 bp, Homo sapiens
tgcagcgcctgcggcct

SEQ ID NO: 470 - DNA, 17 bp, Homo sapiens
ctggggggaagggactg

SEQ ID NO: 471 - DNA, 17 bp, Homo sapiens
gtgctattctggggctg

SEQ ID NO: 472 - DNA, 17 bp, Homo sapiens
ggagggggcttgaagcc

SEQ ID NO: 473 - DNA, 17 bp, Homo sapiens
gtgcctgagaggcaggc

SEQ ID NO: 474 - DNA, 17 bp, Homo sapiens
tcacagggtccccgggg

SEQ ID NO: 475 - DNA, 17 bp, Homo sapiens
gggctccctggccctgg

SEQ ID NO: 476 - DNA, 17 bp, Homo sapiens
gccccaggtagggggac

SEQ ID NO: 477 - DNA, 17 bp, Homo sapiens
gaaagtggctgtcctgg

SEQ ID NO: 478 - DNA, 17 bp, Homo sapiens
tccctggctgttgaggc

SEQ ID NO: 479 - DNA, 17 bp, Homo sapiens
acagagcacagctgccc

SEQ ID NO: 480 - DNA, 17 bp, Homo sapiens
ctttgcactctcctttg

SEQ ID NO: 481 - DNA, 17 bp, Homo sapiens
atgctccctgaggagct

SEQ ID NO: 482 - DNA, 17 bp, Homo sapiens
ccgtccaagggtccgct

SEQ ID NO: 483 - DNA, 17 bp, Homo sapiens
gggccccctgggcagtg

SEQ ID NO: 484 - DNA, 17 bp, Homo sapiens
cttatgctgctggtgcc

SEQ ID NO: 485 - DNA, 17 bp, Homo sapiens
ggttattttggagtgta

SEQ ID NO: 486 - DNA, 17 bp, Homo sapiens
gcctgtccctccaagac

SEQ ID NO: 487 - DNA, 17 bp, Homo sapiens
aagatgagggggcaggc

SEQ ID NO: 488 - DNA, 17 bp, Homo sapiens
ccaacaagaatgcattg

SEQ ID NO: 489 - DNA, 17 bp, Homo sapiens
aaggatgcggtgatggc

SEQ ID NO: 490 - DNA, 17 bp, Homo sapiens
tgtcatcacagacactt

SEQ ID NO: 491 - DNA, 17 bp, Homo sapiens
caggctttttggcttcc

SEQ ID NO: 492 - DNA, 17 bp, Homo sapiens
tcaagttcactgcctgt

SEQ ID NO: 493 - DNA, 17 bp, Homo sapiens
tccctgggcagcttcag

SEQ ID NO: 494 - DNA, 17 bp, Homo sapiens
caggagttcaaagaagg

SEQ ID NO: 495 - DNA, 17 bp, Homo sapiens
caggtggttctgccatc

SEQ ID NO: 496 - DNA, 17 bp, Homo sapiens
gcccacatccgctgagg

SEQ ID NO: 497 - DNA, 17 bp, Homo sapiens
gctggggtgggggtggg

SEQ ID NO: 498 - DNA, 17 bp, Homo sapiens
gacctcctgccctgggg

SEQ ID NO: 499 - DNA, 17 bp, Homo sapiens
agtgaataaatgtcttg

SEQ ID NO: 500 - DNA, 17 bp, Homo sapiens
aaggttcttctcaaggg

SEQ ID NO: 501 - DNA, 17 bp, Homo sapiens
agcctggactgagccac

SEQ ID NO: 502 - DNA, 17 bp, Homo sapiens
caacccagattggggtg

SEQ ID NO: 503 - DNA, 17 bp, Homo sapiens
tgcttctgccaccctgc

SEQ ID NO: 504 - DNA, 17 bp, Homo sapiens
caggtgacaagggccct

SEQ ID NO: 505 - DNA, 17 bp, Homo sapiens
ggccgggggcagttctc

SEQ ID NO: 506 - DNA, 17 bp, Homo sapiens
gtgcgctagggccccgg

SEQ ID NO: 507 - DNA, 17 bp, Homo sapiens
aggctgtccaggctctg

SEQ ID NO: 508 - DNA, 17 bp, Homo sapiens
tgttatgtccattttgc

SEQ ID NO: 509 - DNA, 17 bp, Homo sapiens
tttcccaaactgtgagg

SEQ ID NO: 510 - DNA, 17 bp, Homo sapiens
ggggatggggtactgcc

SEQ ID NO: 511 - DNA, 10 bp, Homo sapiens
gtctcagtgc

SEQ ID NO: 512 - DNA, 10 bp, Homo sapiens
cttatgctgc

SEQ ID NO: 513 - DNA, 10 bp, Homo sapiens
ccaccctcac

SEQ ID NO: 514 - DNA, 10 bp, Homo sapiens
gtgctacttc

SEQ ID NO: 515 - DNA, 10 bp, Homo sapiens
gagtgagacc

SEQ ID NO: 516 - DNA, 10 bp, Homo sapiens
atggcaacag

SEQ ID NO: 517 - DNA, 10 bp, Homo sapiens
tcacacagtg

SEQ ID NO: 518 - DNA, 9 bp, Homo sapiens
gaccgcagg

SEQ ID NO: 519 - DNA, 10 bp, Homo sapiens
gggaggggtg

SEQ ID NO: 520 - DNA, 10 bp, Homo sapiens
ccctaccctg

SEQ ID NO: 521 - DNA, 10 bp, Homo sapiens
ttctcccaaa

SEQ ID NO: 522 - DNA, 10 bp, Homo sapiens
ggatgcgcag

SEQ ID NO: 523 - DNA, 10 bp, Homo sapiens
gtgctaagcg

SEQ ID NO: 524 - DNA, 10 bp, Homo sapiens
cccaggacac

SEQ ID NO: 525 - DNA, 10 bp, Homo sapiens
tagttggaaa

SEQ ID NO: 526 - DNA, 10 bp, Homo sapiens
aagggcgcgg

SEQ ID NO: 527 - DNA, 10 bp, Homo sapiens
agctgtgcca

SEQ ID NO: 528 - DNA, 10 bp, Homo sapiens
acaaaatcaa

SEQ ID NO: 529 - DNA, 10 bp, Homo sapiens
gcctgcagtc

SEQ ID NO: 530 - DNA, 10 bp, Homo sapiens
accaggtcca

SEQ ID NO: 531 - DNA, 10 bp, Homo sapiens
ggctaattat

SEQ ID NO: 532 - DNA, 10 bp, Homo sapiens
tttaaatagc

SEQ ID NO: 533 - DNA, 10 bp, Homo sapiens
cagttcatta

SEQ ID NO: 534 - DNA, 10 bp, Homo sapiens
ctgccgtgac

SEQ ID NO: 535 - DNA, 10 bp, Homo sapiens
ttttaactta

SEQ ID NO: 536 - DNA, 10 bp, Homo sapiens
tagaaaccgg

SEQ ID NO: 537 - DNA, 9 bp, Homo sapiens
cttcttgcc

SEQ ID NO: 538 - DNA, 10 bp, Homo sapiens
tagaaaaaat

SEQ ID NO: 539 - DNA, 10 bp, Homo sapiens
gtgctacttc

SEQ ID NO: 540 - DNA, 10 bp, Homo sapiens
gagtgagacc

SEQ ID NO: 541 - DNA, 10 bp, Homo sapiens
tcacacagtg

SEQ ID NO: 542 - DNA, 10 bp, Homo sapiens
gaccgcagga

SEQ ID NO: 543 - DNA, 10 bp, Homo sapiens
gggaggggtg

SEQ ID NO: 544 - DNA, 10 bp, Homo sapiens
ggggctgccc

SEQ ID NO: 545 - DNA, 10 bp, Homo sapiens
ttctcccaaa

SEQ ID NO: 546 - DNA, 10 bp, Homo sapiens
ccacagggga

SEQ ID NO: 547 - DNA, 10 bp, Homo sapiens
tcaagttcac

SEQ ID NO: 548 - DNA, 10 bp, Homo sapiens
accaaaaacc

SEQ ID NO: 549 - DNA, 10 bp, Homo sapiens
gatcaggcca

SEQ ID NO: 550 - DNA, 10 bp, Homo sapiens
agaaaccacg

SEQ ID NO: 551 - DNA, 10 bp, Homo sapiens
aaggttcttc

SEQ ID NO: 552 - DNA, 10 bp, Homo sapiens
ccctttcaca

SEQ ID NO: 553 - DNA, 10 bp, Homo sapiens
agactagggg

SEQ ID NO: 554 - DNA, 10 bp, Homo sapiens
cataaacggg

SEQ ID NO: 555 - DNA, 10 bp, Homo sapiens
ggccaacatt

SEQ ID NO: 556 - DNA, 10 bp, Homo sapiens
gtacgtccca

SEQ ID NO: 557 - DNA, 10 bp, Homo sapiens
gcaatttaac

SEQ ID NO: 558 - DNA, 10 bp, Homo sapiens
aggtgcgggg

SEQ ID NO: 559 - DNA, 10 bp, Homo sapiens
tttggggctg

SEQ ID NO: 560 - DNA, 10 bp, Homo sapiens
caccctgtac

SEQ ID NO: 561 - DNA, 10 bp, Homo sapiens
gggtgggcgt

SEQ ID NO: 562 - DNA, 17 bp, Homo sapiens
gtacgtcccaccctgtc

SEQ ID NO: 563 - DNA, 17 bp, Homo sapiens
gcaatttaaccacattt

SEQ ID NO: 564 - DNA, 17 bp, Homo sapiens
aggtgcggggggcagac

SEQ ID NO: 565 - DNA, 17 bp, Homo sapiens
tttggggctggcctcac

SEQ ID NO: 566 - DNA, 17 bp, Homo sapiens
caccctgtacagttgcc

SEQ ID NO: 567 - DNA, 17 bp, Homo sapiens
gggtgggcgtgcaggga

SEQ ID NO: 568 - DNA, 17 bp, Homo sapiens
actcagcccggctgatg

SEQ ID NO: 569 - DNA, 17 bp, Homo sapiens
gctctctatgctgacgt

SEQ ID NO: 570 - DNA, 17 bp, Homo sapiens
agaatgaaactgccggg

SEQ ID NO: 571 - DNA, 17 bp, Homo sapiens
aagtggaataaactgcc

SEQ ID NO: 572 - DNA, 17 bp, Homo sapiens
tttgaatcagtgctaga

SEQ ID NO: 573 - DNA, 17 bp, Homo sapiens
ttctgctcttgtgccct

SEQ ID NO: 574 - DNA, 17 bp, Homo sapiens
tgggaagtgggctcctt

SEQ ID NO: 575 - DNA, 17 bp, Homo sapiens
tgggcccgtgtctggcc

SEQ ID NO: 576 - DNA, 17 bp, Homo sapiens
acaacgtccagctggtg

SEQ ID NO: 577 - DNA, 17 bp, Homo sapiens
ccccctgcccctctgcc

SEQ ID NO: 578 - DNA, 17 bp, Homo sapiens
gtctatgcctcccagga

SEQ ID NO: 579 - DNA, 17 bp, Homo sapiens
ggctggagccgctttgg

SEQ ID NO: 580 - DNA, 17 bp, Homo sapiens
catacctcctgccccgc

SEQ ID NO: 581 - DNA, 17 bp, Homo sapiens
tgcctgcaccaggagac

SEQ ID NO: 582 - DNA, 17 bp, Homo sapiens
ttccttgtaatcaaaga

SEQ ID NO: 583 - DNA, 17 bp, Homo sapiens
tggcggcagaggcagag

SEQ ID NO: 584 - DNA, 17 bp, Homo sapiens
ccacgtggctggctggg

SEQ ID NO: 585 - DNA, 17 bp, Homo sapiens
ctggaggctgcctcggg

SEQ ID NO: 586 - DNA, 17 bp, Homo sapiens
atggcaacagatctgga

SEQ ID NO: 587 - DNA, 17 bp, Homo sapiens
ggtcccctacccttccc

SEQ ID NO: 588 - DNA, 17 bp, Homo sapiens
cccgcccccgccttccc

SEQ ID NO: 589 - DNA, 17 bp, Homo sapiens
cccgaggcagagtcggg

SEQ ID NO: 590 - DNA, 17 bp, Homo sapiens
ctacgtgatgaagatgg

SEQ ID NO: 591 - DNA, 17 bp, Homo sapiens
atgggtttgcattttag

SEQ ID NO: 592 - DNA, 17 bp, Homo sapiens
atggaagtctgcgtaac

SEQ ID NO: 593 - DNA, 17 bp, Homo sapiens
tggacagcagggacctg

SEQ ID NO: 594 - DNA, 17 bp, Homo sapiens
tcatagaaaccttgatt

SEQ ID NO: 595 - DNA, 17 bp, Homo sapiens
cagcgctgcattgactc

SEQ ID NO: 596 - DNA, 17 bp, Homo sapiens
ctgggtgccccagcctg

SEQ ID NO: 597 - DNA, 17 bp, Homo sapiens
cgaccccacgccacccc

SEQ ID NO: 598 - DNA, 17 bp, Homo sapiens
cgctgggcgtctgggac

SEQ ID NO: 599 - DNA, 17 bp, Homo sapiens
attagtcagaaactgcc

SEQ ID NO: 600 - DNA, 17 bp, Homo sapiens
gatagcacagttgtcag

SEQ ID NO: 601 - DNA, 17 bp, Homo sapiens
cgtactgagcgctttgg

SEQ ID NO: 602 - DNA, 17 bp, Homo sapiens
ggggcttctgtagcccc

SEQ ID NO: 603 - DNA, 17 bp, Homo sapiens
cccgtccggaacgtcta

SEQ ID NO: 604 - DNA, 17 bp, Homo sapiens
ccagtggcccggagctg

SEQ ID NO: 605 - DNA, 17 bp, Homo sapiens
actgtactccagcctag

SEQ ID NO: 606 - DNA, 17 bp, Homo sapiens
agctccagacccccagc

SEQ ID NO: 607 - DNA, 17 bp, Homo sapiens
gactcgcagacaccggg

SEQ ID NO: 608 - DNA, 17 bp, Homo sapiens
ggcctccagccacccac

SEQ ID NO: 609 - DNA, 17 bp, Homo sapiens
gaccagccttcagatgg

SEQ ID NO: 610 - DNA, 17 bp, Homo sapiens
gcccccaggggcaggac

SEQ ID NO: 611 - DNA, 17 bp, Homo sapiens
gaggatggtgtcctgag

SEQ ID NO: 612 - DNA, 17 bp, Homo sapiens
aagatcaagatcattgc

SEQ ID NO: 613 - DNA, 17 bp, Homo sapiens
agcacagggtcgggggg

SEQ ID NO: 614 - DNA, 17 bp, Homo sapiens
gccaagtgaactgtggc

SEQ ID NO: 615 - DNA, 17 bp, Homo sapiens
cgtgggtggggagggag

SEQ ID NO: 616 - DNA, 17 bp, Homo sapiens
ctcccctatggactggc

SEQ ID NO: 617 - DNA, 17 bp, Homo sapiens
agtatgaggaaatctct

SEQ ID NO: 618 - DNA, 17 bp, Homo sapiens
gcccccttcctcccttg

SEQ ID NO: 619 - DNA, 17 bp, Homo sapiens
gggatttctgtgtctgc

SEQ ID NO: 620 - DNA, 17 bp, Homo sapiens
cttccagctaacaggtc

SEQ ID NO: 621 - DNA, 17 bp, Homo sapiens
gccttgggtgacaaatt

SEQ ID NO: 622 - DNA, 17 bp, Homo sapiens
gcctcccgccacggggc

SEQ ID NO: 623 - DNA, 17 bp, Homo sapiens
gtgtggggggctggggg

SEQ ID NO: 624 - DNA, 17 bp, Homo sapiens
gactcttcagtctggag

SEQ ID NO: 625 - DNA, 17 bp, Homo sapiens
gccacacccaccgcccc

SEQ ID NO: 626 - DNA, 17 bp, Homo sapiens
ccagggcaacagaatga

SEQ ID NO: 627 - DNA, 17 bp, Homo sapiens
caccctgtacagttgcc

SEQ ID NO: 628 - DNA, 17 bp, Homo sapiens
gacgaatatgaatgtca

SEQ ID NO: 629 - DNA, 17 bp, Homo sapiens
caaactggtctaggtca

SEQ ID NO: 630 - DNA, 17 bp, Homo sapiens
gtaatgacagatgcaag

SEQ ID NO: 631 - DNA, 17 bp, Homo sapiens
tggccccaggtgccacc

SEQ ID NO: 632 - DNA, 17 bp, Homo sapiens
aacacaatcatatgatg

SEQ ID NO: 633 - DNA, 17 bp, Homo sapiens
gagggtatactgagggg

SEQ ID NO: 634 - DNA, 17 bp, Homo sapiens
ggagccagctgacctgc

SEQ ID NO: 635 - DNA, 17 bp, Homo sapiens
gagcctcaggtgctccc

SEQ ID NO: 636 - DNA, 17 bp, Homo sapiens
tacttcacatacagtgc

SEQ ID NO: 637 - DNA, 17 bp, Homo sapiens
cggtgggaccaccctgc

SEQ ID NO: 638 - DNA, 17 bp, Homo sapiens
ggagaaacagctgctga

SEQ ID NO: 639 - DNA, 17 bp, Homo sapiens
gctcaggtctgccgggg

SEQ ID NO: 640 - DNA, 17 bp, Homo sapiens
tctgcactgagaaactg

SEQ ID NO: 641 - DNA, 17 bp, Homo sapiens
tccacgcccttcctggc

SEQ ID NO: 642 - DNA, 17 bp, Homo sapiens
ttgcaatagcaaaaccc

SEQ ID NO: 643 - DNA, 17 bp, Homo sapiens
tccaaatcgatgtggat

SEQ ID NO: 644 - DNA, 17 bp, Homo sapiens
agggcttccaatgtgct

SEQ ID NO: 645 - DNA, 17 bp, Homo sapiens
aacctgggaggtggagg

SEQ ID NO: 646 - DNA, 17 bp, Homo sapiens
ggccaacatttggtcca

SEQ ID NO: 647 - DNA, 17 bp, Homo sapiens
ggggctggaggggggca

SEQ ID NO: 648 - DNA, 17 bp, Homo sapiens
ggatgcgcaggggaggc

SEQ ID NO: 649 - DNA, 17 bp, Homo sapiens
gaagacacttggtttga

SEQ ID NO: 650 - DNA, 17 bp, Homo sapiens
gagagaagagtgatctg

SEQ ID NO: 651 - DNA, 17 bp, Homo sapiens
gcagggtggggaggggt

SEQ ID NO: 652 - DNA, 17 bp, Homo sapiens
tcagtgtattaaaaccc

SEQ ID NO: 653 - DNA, 17 bp, Homo sapiens
atactataattgtgaga

SEQ ID NO: 654 - DNA, 17 bp, Homo sapiens
gctggttcctgagtggc

SEQ ID NO: 655 - DNA, 17 bp, Homo sapiens
agccactgcgcccggcc

SEQ ID NO: 656 - DNA, 17 bp, Homo sapiens
agactaggggccggagc

SEQ ID NO: 657 - DNA, 17 bp, Homo sapiens
gggacagctgtctgtgg

SEQ ID NO: 658 - DNA, 17 bp, Homo sapiens
aacccaggaggcggagc

SEQ ID NO: 659 - DNA, 17 bp, Homo sapiens
cagcctgaggctcttgg

SEQ ID NO: 660 - DNA, 17 bp, Homo sapiens
cctcccctgcacctggg

SEQ ID NO: 661 - DNA, 17 bp, Homo sapiens
gcttcagtgggggagag

SEQ ID NO: 662 - DNA, 17 bp, Homo sapiens
tgtttgggggcttttag

SEQ ID NO: 663 - DNA, 17 bp, Homo sapiens
ttttaaattaggttttg

SEQ ID NO: 664 - DNA, 17 bp, Homo sapiens
atctcaaagatacacag

SEQ ID NO: 665 - DNA, 17 bp, Homo sapiens
tttgtgggcagtcaggc

SEQ ID NO: 666 - DNA, 17 bp, Homo sapiens
attgtagacaatgaggg

SEQ ID NO: 667 - DNA, 17 bp, Homo sapiens
gcaaaaccctgctctcc

SEQ ID NO: 668 - DNA, 17 bp, Homo sapiens
gtctcagtgctgaggcg

SEQ ID NO: 669 - DNA, 17 bp, Homo sapiens
agccactgtgcccggcc

SEQ ID NO: 670 - DNA, 17 bp, Homo sapiens
tgattaaggtcggcgct

SEQ ID NO: 671 - DNA, 17 bp, Homo sapiens
ttacaaacagaaaagct

SEQ ID NO: 672 - DNA, 17 bp, Homo sapiens
tttattattgttcaata

SEQ ID NO: 673 - DNA, 17 bp, Homo sapiens
cgggcctcaggtggcag

SEQ ID NO: 674 - DNA, 17 bp, Homo sapiens
taaaggcacagtggctc

SEQ ID NO: 675 - DNA, 17 bp, Homo sapiens
atattaggaagtcgggg

SEQ ID NO: 676 - DNA, 17 bp, Homo sapiens
tgattaaaacaagttgc

SEQ ID NO: 677 - DNA, 17 bp, Homo sapiens
ttgtgcacgcatcagtg

SEQ ID NO: 678 - DNA, 17 bp, Homo sapiens
cctgcctcgtagtgaag

SEQ ID NO: 679 - DNA, 17 bp, Homo sapiens
caaaactgtttgttggc

SEQ ID NO: 680 - DNA, 17 bp, Homo sapiens
taggaaagtaaaatgga

SEQ ID NO: 681 - DNA, 17 bp, Homo sapiens
ctccatcggctgtgagg

SEQ ID NO: 682 - DNA, 17 bp, Homo sapiens
caagcatccccgttcca

SEQ ID NO: 683 - DNA, 17 bp, Homo sapiens
gagttaggcacttcctg

SEQ ID NO: 684 - DNA, 17 bp, Homo sapiens
cataaacgggcacaccc

SEQ ID NO: 685 - DNA, 17 bp, Homo sapiens
ttgcctgggatgctggt

SEQ ID NO: 686 - DNA, 17 bp, Homo sapiens
aacgcggccaatgtggg

SEQ ID NO: 687 - DNA, 17 bp, Homo sapiens
ggtttggcttaggctgg

SEQ ID NO: 688 - DNA, 17 bp, Homo sapiens
gatttttgtggtgtggg

SEQ ID NO: 689 - DNA, 17 bp, Homo sapiens
ggctgccctgggcagcc

SEQ ID NO: 690 - DNA, 17 bp, Homo sapiens
atggcaacagaaaccaa

SEQ ID NO: 691 - DNA, 17 bp, Homo sapiens
agagcaaaccgtagtcc

SEQ ID NO: 692 - DNA, 17 bp, Homo sapiens
tgcacttcaagaaaatg

SEQ ID NO: 693 - DNA, 17 bp, Homo sapiens
ccctaccctgttacctt

SEQ ID NO: 694 - DNA, 17 bp, Homo sapiens
ggaaaaatgttggaatg

SEQ ID NO: 695 - DNA, 17 bp, Homo sapiens
gtttttgcttcagcggc

SEQ ID NO: 696 - DNA, 17 bp, Homo sapiens
gaggacccaacaggagg

SEQ ID NO: 697 - DNA, 17 bp, Homo sapiens
actgaggaaaggagctc

SEQ ID NO: 698 - DNA, 17 bp, Homo sapiens
tgcagcgcctgcggcct

SEQ ID NO: 699 - DNA, 17 bp, Homo sapiens
ctggggggaagggactg

SEQ ID NO: 700 - DNA, 17 bp, Homo sapiens
gtgcctgagaggcaggc

SEQ ID NO: 701 - DNA, 17 bp, Homo sapiens
tcacagggtccccgggg

SEQ ID NO: 702 - DNA, 17 bp, Homo sapiens
ggagggggcttgaagcc

SEQ ID NO: 703 - DNA, 17 bp, Homo sapiens
ggaggaattcatcttca

SEQ ID NO: 704 - DNA, 17 bp, Homo sapiens
gaaagtggctgtcctgg

SEQ ID NO: 705 - DNA, 17 bp, Homo sapiens
tccctggctgttgaggc

SEQ ID NO: 706 - DNA, 17 bp, Homo sapiens
aagatgagggggcaggc

SEQ ID NO: 707 - DNA, 17 bp, Homo sapiens
ccaacaagaatgcattg

SEQ ID NO: 708 - DNA, 17 bp, Homo sapiens
aaggatgcggtgatggc

SEQ ID NO: 709 - DNA, 17 bp, Homo sapiens
tgcagtcactggtgtca

SEQ ID NO: 710 - DNA, 17 bp, Homo sapiens
gccgtgtagaccctaac

SEQ ID NO: 711 - DNA, 17 bp, Homo sapiens
caggctttttggcttcc

SEQ ID NO: 712 - DNA, 17 bp, Homo sapiens
tccctgggcagcttcag

SEQ ID NO: 713 - DNA, 17 bp, Homo sapiens
gagcgcagcgagctagc

SEQ ID NO: 714 - DNA, 17 bp, Homo sapiens
caggtggttctgccatc

SEQ ID NO: 715 - DNA, 17 bp, Homo sapiens
gcccacatccgctgagg

SEQ ID NO: 716 - DNA, 17 bp, Homo sapiens
aaggttcttctcaaggg

SEQ ID NO: 717 - DNA, 10 bp, Homo sapiens
actcagcccg

SEQ ID NO: 718 - DNA, 10 bp, Homo sapiens
gctctctatg

SEQ ID NO: 719 - DNA, 10 bp, Homo sapiens
agaatgaaac

SEQ ID NO: 720 - DNA, 10 bp, Homo sapiens
aagtggaata

SEQ ID NO: 721 - DNA, 10 bp, Homo sapiens
tttgaatcag

SEQ ID NO: 722 - DNA, 10 bp, Homo sapiens
ttctgctctt

SEQ ID NO: 723 - DNA, 10 bp, Homo sapiens
tgggaagtgg

SEQ ID NO: 724 - DNA, 10 bp, Homo sapiens
tgggcccgtg

SEQ ID NO: 725 - DNA, 10 bp, Homo sapiens
acaacgtcca

SEQ ID NO: 726 - DNA, 10 bp, Homo sapiens
ccccctgccc

SEQ ID NO: 727 - DNA, 10 bp, Homo sapiens
gtctatgcct

SEQ ID NO: 728 - DNA, 10 bp, Homo sapiens
ggctggagcc

SEQ ID NO: 729 - DNA, 10 bp, Homo sapiens
catacctcct

SEQ ID NO: 730 - DNA, 10 bp, Homo sapiens
tgcctgcacc

SEQ ID NO: 731 - DNA, 10 bp, Homo sapiens
ttccttgtaa

SEQ ID NO: 732 - DNA, 10 bp, Homo sapiens
tggcggcaga

SEQ ID NO: 733 - DNA, 10 bp, Homo sapiens
ccacgtggct

SEQ ID NO: 734 - DNA, 10 bp, Homo sapiens
ctggaggctg

SEQ ID NO: 735 - DNA, 10 bp, Homo sapiens
atggcaacag

SEQ ID NO: 736 - DNA, 10 bp, Homo sapiens
ggtcccctac

SEQ ID NO: 737 - DNA, 10 bp, Homo sapiens
cccgcccccg

SEQ ID NO: 738 - DNA, 10 bp, Homo sapiens
cccgaggcag

SEQ ID NO: 739 - DNA, 10 bp, Homo sapiens
ctacgtgatg

SEQ ID NO: 740 - DNA, 10 bp, Homo sapiens
atgggtttgc

SEQ ID NO: 741 - DNA, 10 bp, Homo sapiens
atggaagtct

SEQ ID NO: 742 - DNA, 10 bp, Homo sapiens
tggacagcag

SEQ ID NO: 743 - DNA, 10 bp, Homo sapiens
tcatagaaac

SEQ ID NO: 744 - DNA, 10 bp, Homo sapiens
cagcgctgca

SEQ ID NO: 745 - DNA, 10 bp, Homo sapiens
ctgggtgccc

SEQ ID NO: 746 - DNA, 10 bp, Homo sapiens
cgaccccacg

SEQ ID NO: 747 - DNA, 10 bp, Homo sapiens
cgctgggcgt

SEQ ID NO: 748 - DNA, 10 bp, Homo sapiens
attagtcaga

SEQ ID NO: 749 - DNA, 10 bp, Homo sapiens
gatagcacag

SEQ ID NO: 750 - DNA, 10 bp, Homo sapiens
cgtactgagc

SEQ ID NO: 751 - DNA, 10 bp, Homo sapiens
ggggcttctg

SEQ ID NO: 752 - DNA, 10 bp, Homo sapiens
cccgtccgga

SEQ ID NO: 753 - DNA, 10 bp, Homo sapiens
ccagtggccc

SEQ ID NO: 754 - DNA, 10 bp, Homo sapiens
actgtactcc

SEQ ID NO: 755 - DNA, 10 bp, Homo sapiens
agctccagac

SEQ ID NO: 756 - DNA, 10 bp, Homo sapiens
gactcgcaga

SEQ ID NO: 757 - DNA, 10 bp, Homo sapiens
ggcctccagc

SEQ ID NO: 758 - DNA, 10 bp, Homo sapiens
gaccagcctt

SEQ ID NO: 759 - DNA, 10 bp, Homo sapiens
gcccccaggg

SEQ ID NO: 760 - DNA, 10 bp, Homo sapiens
gaggatggtg

SEQ ID NO: 761 - DNA, 10 bp, Homo sapiens
aagatcaaga

SEQ ID NO: 762 - DNA, 10 bp, Homo sapiens
agcacagggt

SEQ ID NO: 763 - DNA, 10 bp, Homo sapiens
gccaagtgaa

SEQ ID NO: 764 - DNA, 10 bp, Homo sapiens
cgtgggtggg

SEQ ID NO: 765 - DNA, 10 bp, Homo sapiens
ctcccctatg

SEQ ID NO: 766 - DNA, 10 bp, Homo sapiens
agtatgagga

SEQ ID NO: 767 - DNA, 10 bp, Homo sapiens
gcccccttcc

SEQ ID NO: 768 - DNA, 10 bp, Homo sapiens
gggatttctg

SEQ ID NO: 769 - DNA, 10 bp, Homo sapiens
cttccagcta

SEQ ID NO: 770 - DNA, 10 bp, Homo sapiens
gccttgggtg

SEQ ID NO: 771 - DNA, 10 bp, Homo sapiens
gcctcccgcc

SEQ ID NO: 772 - DNA, 10 bp, Homo sapiens
gtgtgggggg

SEQ ID NO: 773 - DNA, 10 bp, Homo sapiens
gactcttcag

SEQ ID NO: 774 - DNA, 10 bp, Homo sapiens
gccacaccca

SEQ ID NO: 775 - DNA, 10 bp, Homo sapiens
ccagggcaac

SEQ ID NO: 776 - DNA, 10 bp, Homo sapiens
caccctgtac

SEQ ID NO: 777 - DNA, 10 bp, Homo sapiens
gacgaatatg

SEQ ID NO: 778 - DNA, 10 bp, Homo sapiens
caaactggtc

SEQ ID NO: 779 - DNA, 10 bp, Homo sapiens
gtaatgacag

SEQ ID NO: 780 - DNA, 10 bp, Homo sapiens
tggccccagg

SEQ ID NO: 781 - DNA, 10 bp, Homo sapiens
aacacaatca

SEQ ID NO: 782 - DNA, 10 bp, Homo sapiens
gagggtatac

SEQ ID NO: 783 - DNA, 10 bp, Homo sapiens
ggagccagct

SEQ ID NO: 784 - DNA, 10 bp, Homo sapiens
gagcctcagg

SEQ ID NO: 785 - DNA, 10 bp, Homo sapiens
tacttcacat

SEQ ID NO: 786 - DNA, 10 bp, Homo sapiens
cggtgggacc

SEQ ID NO: 787 - DNA, 10 bp, Homo sapiens
ggagaaacag

SEQ ID NO: 788 - DNA, 10 bp, Homo sapiens
gctcaggtct

SEQ ID NO: 789 - DNA, 10 bp, Homo sapiens
tctgcactga

SEQ ID NO: 790 - DNA, 10 bp, Homo sapiens
tccacgccct

SEQ ID NO: 791 - DNA, 10 bp, Homo sapiens
ttgcaatagc

SEQ ID NO: 792 - DNA, 10 bp, Homo sapiens
tccaaatcga

SEQ ID NO: 793 - DNA, 10 bp, Homo sapiens
agggcttcca

SEQ ID NO: 794 - DNA, 10 bp, Homo sapiens
aacctgggag

SEQ ID NO: 795 - DNA, 10 bp, Homo sapiens
ggccaacatt

SEQ ID NO: 796 - DNA, 10 bp, Homo sapiens
ggggctggag

SEQ ID NO: 797 - DNA, 10 bp, Homo sapiens
ggatgcgcag

SEQ ID NO: 798 - DNA, 10 bp, Homo sapiens
gaagacactt

SEQ ID NO: 799 - DNA, 10 bp, Homo sapiens
gagagaagag

SEQ ID NO: 800 - DNA, 10 bp, Homo sapiens
gcagggtggg

SEQ ID NO: 801 - DNA, 10 bp, Homo sapiens
tcagtgtatt

SEQ ID NO: 802 - DNA, 10 bp, Homo sapiens
atactataat

SEQ ID NO: 803 - DNA, 10 bp, Homo sapiens
gctggttcct

SEQ ID NO: 804 - DNA, 10 bp, Homo sapiens
agccactgcg

SEQ ID NO: 805 - DNA, 10 bp, Homo sapiens
agactagggg

SEQ ID NO: 806 - DNA, 10 bp, Homo sapiens
gggacagctg

SEQ ID NO: 807 - DNA, 10 bp, Homo sapiens
aacccaggag

SEQ ID NO: 808 - DNA, 10 bp, Homo sapiens
cagcctgagg

SEQ ID NO: 809 - DNA, 10 bp, Homo sapiens
cctcccctgc

SEQ ID NO: 810 - DNA, 10 bp, Homo sapiens
gcttcagtgg

SEQ ID NO: 811 - DNA, 10 bp, Homo sapiens
tgtttggggg

SEQ ID NO: 812 - DNA, 10 bp, Homo sapiens
ttttaaatta

SEQ ID NO: 813 - DNA, 10 bp, Homo sapiens
atctcaaaga

SEQ ID NO: 814 - DNA, 10 bp, Homo sapiens
tttgtgggca

SEQ ID NO: 815 - DNA, 10 bp, Homo sapiens
attgtagaca

SEQ ID NO: 816 - DNA, 10 bp, Homo sapiens
gcaaaaccct

SEQ ID NO: 817 - DNA, 10 bp, Homo sapiens
gtctcagtgc

SEQ ID NO: 818 - DNA, 10 bp, Homo sapiens
agccactgtg

SEQ ID NO: 819 - DNA, 10 bp, Homo sapiens
tgattaaggt

SEQ ID NO: 820 - DNA, 10 bp, Homo sapiens
ttacaaacag

SEQ ID NO: 821 - DNA, 10 bp, Homo sapiens
tttattattg

SEQ ID NO: 822 - DNA, 10 bp, Homo sapiens
cgggcctcag

SEQ ID NO: 823 - DNA, 10 bp, Homo sapiens
taaaggcaca

SEQ ID NO: 824 - DNA, 10 bp, Homo sapiens
atattaggaa

SEQ ID NO: 825 - DNA, 10 bp, Homo sapiens
tgattaaaac

SEQ ID NO: 826 - DNA, 10 bp, Homo sapiens
ttgtgcacgc

SEQ ID NO: 827 - DNA, 10 bp, Homo sapiens
cctgcctcgt

SEQ ID NO: 828 - DNA, 10 bp, Homo sapiens
caaaactgtt

SEQ ID NO: 829 - DNA, 10 bp, Homo sapiens
taggaaagta

SEQ ID NO: 830 - DNA, 10 bp, Homo sapiens
ctccatcggc

SEQ ID NO: 831 - DNA, 10 bp, Homo sapiens
caagcatccc

SEQ ID NO: 832 - DNA, 10 bp, Homo sapiens
gagttaggca

SEQ ID NO: 833 - DNA, 10 bp, Homo sapiens
cataaacggg

SEQ ID NO: 834 - DNA, 10 bp, Homo sapiens
ttgcctggga

SEQ ID NO: 835 - DNA, 10 bp, Homo sapiens
aacgcggcca

SEQ ID NO: 836 - DNA, 10 bp, Homo sapiens
ggtttggctt

SEQ ID NO: 837 - DNA, 10 bp, Homo sapiens
gatttttgtg

SEQ ID NO: 838 - DNA, 10 bp, Homo sapiens
ggctgccctg

SEQ ID NO: 839 - DNA, 10 bp, Homo sapiens
atggcaacag

SEQ ID NO: 840 - DNA, 10 bp, Homo sapiens
agagcaaacc

SEQ ID NO: 841 - DNA, 10 bp, Homo sapiens
tgcacttcaa

SEQ ID NO: 842 - DNA, 10 bp, Homo sapiens
ccctaccctg

SEQ ID NO: 843 - DNA, 10 bp, Homo sapiens
ggaaaaatgt

SEQ ID NO: 844 - DNA, 10 bp, Homo sapiens
gtttttgctt

SEQ ID NO: 845 - DNA, 10 bp, Homo sapiens
gaggacccaa

SEQ ID NO: 846 - DNA, 10 bp, Homo sapiens
actgaggaaa

SEQ ID NO: 847 - DNA, 10 bp, Homo sapiens
tgcagcgcct

SEQ ID NO: 848 - DNA, 10 bp, Homo sapiens
ctggggggaa

SEQ ID NO: 849 - DNA, 10 bp, Homo sapiens
gtgcctgaga

SEQ ID NO: 850 - DNA, 10 bp, Homo sapiens
tcacagggtc

SEQ ID NO: 851 - DNA, 10 bp, Homo sapiens
ggagggggct

SEQ ID NO: 852 - DNA, 10 bp, Homo sapiens
ggaggaattc

SEQ ID NO: 853 - DNA, 10 bp, Homo sapiens
gaaagtggct

SEQ ID NO: 854 - DNA, 10 bp, Homo sapiens
tccctggctg

SEQ ID NO: 855 - DNA, 10 bp, Homo sapiens
aagatgaggg

SEQ ID NO: 856 - DNA, 10 bp, Homo sapiens
ccaacaagaa

SEQ ID NO: 857 - DNA, 10 bp, Homo sapiens
aaggatgcgg

SEQ ID NO: 858 - DNA, 10 bp, Homo sapiens
tgcagtcact

SEQ ID NO: 859 - DNA, 10 bp, Homo sapiens
gccgtgtaga

SEQ ID NO: 860 - DNA, 10 bp, Homo sapiens
caggcttttt

SEQ ID NO: 861 - DNA, 10 bp, Homo sapiens
tccctgggca

SEQ ID NO: 862 - DNA, 10 bp, Homo sapiens
gagcgcagcg

SEQ ID NO: 863 - DNA, 10 bp, Homo sapiens
caggtggttc

SEQ ID NO: 864 - DNA, 10 bp, Homo sapiens
gcccacatcc

SEQ ID NO: 865 - DNA, 10 bp, Homo sapiens
aaggttcttc

SEQ ID NO: 866 - DNA, 10 bp, Homo sapiens
ggctaattat

SEQ ID NO: 867 - DNA, 10 bp, Homo sapiens
caaaaataaa

SEQ ID NO: 868 - DNA, 17 bp, Homo sapiens
ggctaattatcatcaat

SEQ ID NO: 869 - DNA, 16 bp, Homo sapiens
caaaaataaaagccga

(19) United States
(12) Patent Application Publication
(10) Pub. No.: US 2012/0308479 A1
(43) Pub. Date: Dec. 6, 2012

(54) Brain Endothelial Cell Expression Patterns

(75) Inventors: Stephen MADDEN, Sudbury, MA (US); Clarence WANG, Arlington, MA (US); Brian P. COOK, Northboro, MA (US); John LATERRA, Baltimore, MD (US); Kevin WALTER, (US)

(73) Assignees: THE JOHNS HOPKINS UNIVERSITY, Baltimore, MD (US); GENZYME CORPORATION, Framingham, MA (US)

(21) Appl. No.: 13/544,631

(22) Filed: Jul. 9, 2012

Related U.S. Application Data
(63) Continuation of application No. 12/616,600, filed on Nov. 11, 2009, now abandoned.
Application No. 12/616,600 is a division of application No. 10/524,432, filed on Sep. 9, 2005, now abandoned.
(60) Provisional application No. 60/403,390, filed on Aug. 15, 2002.
(60) Provisional application No. 60/458,978, filed on Apr. 1, 2003.

(51) Int. Cl. C40B 30/04; C12Q 1/68; A61K 39/395; A61K 31/7088; A61K 51/10; A61K 39/00; A61P 37/04; G01N 33/574; A61P 35/00

(52) U.S. Cl. 424/1.49; 506/9; 435/7.23; 435/6.11; 435/6.12; 435/7.4; 435/7.1; 435/6.13; 435/6.19; 424/174.1; 424/158.1; 424/178.1; 424/277.1

(57) ABSTRACT

To gain a better understanding of brain tumor angiogenesis, new techniques for isolating brain endothelial cells (ECs) and evaluating gene expression patterns were developed. When transcripts from brain ECs derived from normal and malignant colorectal tissues were compared with transcripts from non-endothelial cells, genes predominantly expressed in the endothelium were identified. Comparison between normal- and tumor-derived endothelium revealed genes that were specifically elevated in tumor-associated brain endothelium. These results confirm that neoplastic and normal endothelium in human brains are distinct at the molecular level, and have significant implications for the development of anti-angiogenic therapies in the future.

This application claims the benefit of provisional application Ser. Nos. 60/403,390 filed Aug. 15, 2002 and 60/458,978 filed Apr. 1, 2003. The disclosures of each are expressly incorporated herein.

### TECHNICAL FIELD OF THE INVENTION

This invention is related to the area of angiogenesis and anti-angiogenesis. In particular, it relates to genes which are characteristically expressed in brain glioma endothelial cells.

### BACKGROUND OF THE INVENTION

Brain cancers represent an infrequent but deadly form of cancer that has seen little improvement in survivability over the last 30 years. Tumor excision followed by therapies relying on outdated cytotoxins and radiation inevitably results in a diminished quality of life. Gliomas represent the most common brain neoplasms with highly vascular and invasive characteristics defining gliomas as one of the most aggressive tumors known. Classifications of gliomas derive from both the cellular origin and staged aggressiveness. Derived from either astrocytes or oligodendrocytes, astrocytomas and oligodendrogliomas constitute the most common types of gliomas. As is common to other tumor type classifications, glioma increases in aggressiveness from the first to third stages of disease with stage 1V, gliobastoma multiforme, being the most aggressive. Moreover, glioblastoma tumors constitute one of the most vascular tumors known.

Vascular permeability within the brain is limited in comparison to other organs. Similarly, the accessibility of brain malignancies to immune surveillance was thought to be restricted as well although more recent evidence suggests the brain is not wholly immunologically privileged. This so called “blood-brain barrier” is thought to derive primarily from a combination of brain-specific capillary transport systems and astrocyte-regulated cross-talk with the endothelial cell-based vasculature (for reviews, see Bart, J., Groen, H. J., Hendrikse, N. H., van der Graaf, W. T., Vaalburg, W., and de Vries, E. G. (2000). The blood-brain barrier and oncology: new insights into function and modulation. Cancer Treat Rev 26, 449-62.) The presence of tight junctions and an observed high electrical resistance both contribute to restricted transvascular molecular exchange. The existence of a therapeutically impermeable vasculature has resulted in a comparatively limited amount of work aimed at intervening in brain malignancies and other CNS diseases. Defining proteins preferentially expressed on either normal or diseased brain endothelial cells holds promise for expanding CNS therapeutic regimens.

The vascular microenvironment within gliomas has been studied primarily through morphological, circulatory and perfusion based experiments (for review see Vajkoczy, P., and Menger, M. D. (2000). Vascular microenvironment in gliomas. J Neurooncol 50, 99-108; and Bart, J., Groen, H. J., Hendrikse, N. H., van der Graaf, W. T., Vaalburg, W., and de Vries, E. G. (2000). The blood-brain barrier and oncology: new insights into function and modulation. Cancer Treat Rev 26, 449-62.) These studies demonstrate profound changes in vasculature architecture associated with tumor progression. Increased fenestrations, malperfusion, hyperpermeability, and reduced leukocyte-EC interaction are all phenotypic observations linked to glioma microvasculature Bernsen, H. J., Rijken, P. F., Oostendorp, T., and van der Kogel, A. J. (1995). Vascularity and perfusion of human gliomas xenografted in the athymic nude mouse. Br J Cancer 71, 721-6; Vick, N. A., and Bigner, D. D. (1972). Microvascular abnormalities in virally-induced canine brain tumors. Structural bases for altered blood-brain barrier function. J Neurol Sci 17, 29-39; and Hobbs, S. K., Monsky, W. L., Yuan, F., Roberts, W. G., Griffith, L., Torchilin, V. P., and Jain, R. K. (1998). Regulation of transport pathways in tumor vessels: role of tumor type and microenvironment. Proc Natl Acad Sci USA 95, 4607-12. It is also suggested that higher grade gliomas utilize intussuceptive capillary growth to a much larger degree than earlier staged gliomas that primarily utilize both sprouting an cooption to advance vessel growth. Vajkoczy, P., Schilling, L., Ullrich, A., Schmiedek, P., and Menger, M. D. (1998). Characterization of angiogenesis and microcirculation of high-grade glioma: an intravital multifluorescence microscopic approach in the athymic nude mouse. J Cereb Blood Flow Metab 18, 510-20. The molecular characterization of glioma ECs has thus far been limited to the evaluation of common growth factors or previously defined brain EC transporters. Holash, J., Maisonpierre, P. C., Compton, D., Boland, P., Alexander, C. R., Zagzag, D., Yancopoulos, G. D., and Wiegand, S. J. (1999). Vessel cooption, regression, and growth in tumors mediated by angiopoietins and VEGF. Science 284, 1994-8; Guerin, C., Wolff, J. E., Laterra, J., Drewes, L. R., Brem, H., and Goldstein, G. W. (1992). Vascular differentiation and glucose transporter expression in rat gliomas: effects of steroids. Ann Neurol 31, 481-7.

To date, global gene expression profiles from endothelial cell-specific populations is limited to normal and tumorigenic colon tissue. St Croix, B., Rago, C., Velculescu, V., Traverso, G., Romans, K. E., Montgomery, E., Lal, A., Riggins, G. J., Lengauer, C., Vogelstein, B., and Kinzler, K. W. (2000). Genes expressed in human tumor endothelium. Science 289, 1197-202. There is a need in the art for analysis of endothelial cells from other tissue, so that diagnostic and therapeutic for non-colonic tumors can be developed.

### SUMMARY OF THE INVENTION

According to one embodiment of the invention a method is provided to aid in diagnosing glioma. An expression product of at least one gene in a first brain tissue sample suspected of being neoplastic is detected. The at least one gene is selected from the group consisting of signal sequence receptor, delta (translocon-associated protein delta); DC2 protein; KIAA0404 protein; symplekin; Huntingtin interacting protein I; plasmalemma vesicle associated protein; KIAA0726 gene product; latexin protein; transforming growth factor, beta 1; hypothetical protein FLJ22215; Rag C protein; hypothetical protein FLJ23471; N-myristoyltransferase 1; hypothetical protein dJ1181N3.1; ribosomal protein L27; secreted protein, acidic, cysteine-rich (osteonectin); Hs 111988; Hs 112238; laminin, alpha 5; protective protein for beta-galactosidase (galactosialidosis); Melanoma associated gene; Melanoma associated gene; E3 ubiquitin ligase SMURF1; collagen, type N, alpha 1; collagen, type IV, alpha 1; collagen, type IV, alpha 1; insulin-like growth factor binding protein 7; gene predicted from cDNA with a complete coding sequence; Thy-1 cell surface antigen; Hs 127824; GTP binding protein 2; Homo sapiens mRNA; cDNA DKFZp586D0918 (from clone DKFZp586D0918); cutaneous T-cell lymphoma-associated tumor antigen se20-4; differentially expressed nucleolar TGF-beta1 target protein (DENTT); dysferlin, limb girdle muscular dystrophy 2B (autosomal recessive); smoothelin; integrin, alpha 5 (fibronectin receptor, alpha polypeptide); putative translation initiation factor; retinoic acid induced 14; matrix metalloproteinase 9 (gelatinase B, 92 kD gelatinase, 92 kD type IV collagenase); Lutheran blood group (Auberger b antigen included); stanniocalcin 2; nuclear factor (erythroid-derived 2)-like 2; protein tyrosine phosphatase, non-receptor type 1; integrin, alpha 10; collagen, type VI, alpha 2; chromosome 21 open reading frame 25; CDC37 (cell division cycle 37, S. cerevisiae, homolog); Hs 16450; Rho guanine nucleotide exchange factor (GEF) 7; creatine kinase, brain; hypothetical protein FLJ10297; hypothetical protein FLJ10350; TNF-induced protein; tumor necrosis factor receptor superfamily, member 12 (translocating chain-association membrane protein); cofilin 1 (non-muscle); splicing factor proline/glutamine rich (polypyrimidine tract-binding protein-associated); splicing factor proline/glutamine rich (polypyrimidine tract-binding protein-associated); v-ets avian erythroblastosis virus E26 oncogene homolog 1; protease, cysteine, 1 (legumain); ribosomal protein L13; chromosome 22 open reading frame 5; zinc finger protein 144 (MeI-18); degenerative spermatocyte (homolog Drosophila; lipid desaturase); eukaryotic translation initiation factor 2C, 2; mitochondrial ribosomal protein 145; prostate tumor over expressed gene 1; NADH dehydrogenase (ubiquinone) 1 alpha subcomplex, 7 (14.5 kD, B14.5a); glioma endothelial marker 1 precursor; NS1-binding protein; ribosomal protein L38; tuftelin-interacting protein; HLA class II region expressed gene KE2; translocase of inner mitochondrial membrane 17 homolog A (yeast); sudD (suppressor of bimD6, Aspergillus nidulans) homolog; heparan sulfate proteoglycan 2 (perlecan); SEC24 (S. cerevisiae) related gene family, member A; NADH dehydrogenase (ubiquinone) Fe—S protein 7 (20 kD) (NADH-coenzyme Q reductase); DNA segment on chromosome X and Y (unique) 155 expressed sequence; annexin A2; Homo sapiens clone 24670 mRNA sequence; hypothetical protein; matrix metalloproteinase 10 (stromelysin 2); KIAA1049 protein; G protein-coupled receptor; hypothetical protein FLJ20401; matrix metalloproteinase 14 (membrane-inserted); KIAA0470 gene product; solute carrier family 29 (nucleoside transporters), member 1; stanniocalcin 1; stanniocalcin 1; stanniocalcin 1; tumor suppressor deleted in oral cancer-related 1; tumor suppressor deleted in oral cancer-related 1; apolipoprotein C-I; glutathione peroxidase 4 (phospholipid hydroperoxidase); Hs 272106; transcription factor binding to IGHM enhancer 3; hypothetical protein DKFZp762A227; hypothetical protein FLJ22362; CD59 antigen p18-20 (antigen identified by monoclonal antibodies 16.3A5, EJ16, EJ30, EL32 and G344); PRO0628 protein; melanoma-associated antigen recognised by cytotoxic T lymphocytes; LOC88745; Homo sapiens beta-1,3-galactosyltransferase-6 (B3GALT6) mRNA, complete cds; sprouty (Drosophila) homolog 4; sprouty (Drosophila) homolog 4; Homo sapiens mRNA; cDNA DKFZp434E1515 (from clone DKFZp434E1515); coactosin-like protein; hypothetical protein FLJ21865; Hs296234; KIAA0685 gene product; hypothetical protein FLJ10980; ribosomal protein L10; ribosomal protein S19; Hs 299251; Huntingtin interacting protein K; Homo sapiens mRNA full length insert cDNA clone EUROIMAGE 50374; Hs 311780; Hs 212191; v-akt murine thymoma viral oncogene homolog 2; Hs 328774; transducin-like enhancer of split 2, homolog of Drosophila E(sp1); KIAA1870 protein; ribosomal protein L10a; peptidylprolyl isomerase A (cyclophilin A); Hs 344224; hypothetical protein FLJ23239; hypothetical protein DKFZp761H221; KIAA1887 protein; Homo sapiens mRNA full length insert cDNA clone EUROIMAGE 701679; Homo sapiens cDNA FLJ30634 fis, clone CTONG2002453; Homo sapiens cDNA FLJ32203 fis, clone PLACE6003038, weakly similar to ZINC FINGER PROTEIN 84; Homo sapiens mRNA full length insert cDNA clone EUROIMAGE 1035904; hypothetical protein L0057333; myosin ID; plexin B2; lectin, galactoside-binding, soluble, 8 (galectin 8); double ring-finger protein, Dorfin; DKFZP434B168 protein; LIM domain binding 2; integrin beta 4 binding protein; synaptopodin; Hs 54828; insulin induced gene 1; acetyl LDL receptor; SREC; excision repair cross-complementing rodent repair deficiency, complementation group 1 (includes overlapping antisense sequence); hypothetical protein FLJ22329; schwannomin-interacting protein 1; PTEN induced putative kinase 1; myosin X; Homo sapiens cDNA FLJ32424 fis, clone SKMUS2000954, moderately similar to Homo sapiens F-box protein Fbx25 (FBX25) 97; golgi phosphoprotein 1; splicing factor, arginine/serine-rich 6; laminin, gamma 3; cysteine-rich protein 2; U6 snRNA-associated Sm-like protein LSm7; hypothetical protein FLJ10707; Homo sapiens, Similar to RIKEN cDNA 2310012N15 gene, clone IMAGE:3342825, mRNA, partial cds; macrophage migration inhibitory factor (glycosylation-inhibiting factor); ubiquinol-cytochrome c reductase hinge protein; gap junction protein, alpha 1, 43 kD (connexin 43); dihydropyrimidinase-like 3; aquaporin 1 (channel-forming integral protein, 28 kD); protein expressed in thyroid; macrophage myristoylated alanine-rich C kinase substrate; procollagen-lysine, 2-oxoglutarate 5-dioxygenase (lysine hydroxylase, Ehlers-Danlos syndrome type VI); protease, serine, 11 (IGF binding); 24-dehydrocholesterol reductase; collagen, type IV, alpha 2; profilin 1; apolipoprotein D; hyaluronoglucosaminidase 2; hypothetical protein FLJ22678; quiescin Q6; ras homolog gene family, member A; ras homolog gene family, member A; plasminogen activator, urokinase; insulin-like growth factor binding protein 3; uridine phosphorylase; KIAA0638 protein; B7 homolog 3; lamin A/C; lamin A/C; lamin A/C; regulator of G-protein signalling 12; proteasome (prosome, macropain) 26S subunit, non-ATPase, 8; Homo sapiens, Similar to RIKEN cDNA 5730528L13 gene, clone MGC:17337 IMAGE:4213591, mRNA, complete cds; prosaposin (variant Gaucher disease and variant metachromatic leukodystrophy); laminin, alpha 4; transcription elongation factor A (SII), 1; lectin, galactoside-binding, soluble, 3 binding protein; ribosomal protein S16; glycophorin C (Gerbich blood group); endothelin receptor type B; serine (or cysteine) proteinase inhibitor, clade E (nexin, plasminogen activator inhibitor type 1), member 1; biglycan; small nuclear ribonucleoprotein polypeptide B″; transmembrane 4 superfamily member 2; TAF11 RNA polymerase II, TATA box binding protein (TBP)-associated factor, 28 kD; lysyl oxidase-like 2; SRY (sex determining region Y)-box 4; SOX4 SRY (sex determining region Y)-box 4; SRY (sex determining region Y)-box 4; actin related protein 2/3 complex, subunit 2 (34 kD); Homo sapiens cDNA: FLJ23507 fis, clone LNG03128; hypothetical protein FLJ12442; Fas (TNFRSF6)-associated via death domain; mitogen-activated protein kinase kinase kinase 11; TEK tyrosine kinase, endothelial (venous malformations, multiple cutaneous and mucosal); insulin receptor; cell membrane glycoprotein, 110000M(r) (surface antigen); Homo sapiens cDNA FLJ11863 fis, clone HEMBA1006926; jagged 1 (Alagille syndrome); KIAA0304 gene product; pre-B-cell leukemia transcription factor 2; Homo sapiens cDNA FLJ31238 fis, clone KIDNE2004864; p53-induced protein; complement component 1, q subcomponent, receptor 1; complement component 1, q subcomponent, receptor 1; apolipoprotein E; chemokine (C—C motif) ligand 3; coagulation factor II (thrombin) receptor-like 3; coagulation factor III (thromboplastin, tissue factor); collagen, type I, alpha 1; collagen, type III, alpha 1 (Ehlers-Danlos syndrome type IV, autosomal dominant); C-type (calcium dependent, carbohydrate-recognition domain) lectin, superfamily member 9; cystatin C (amyloid angiopathy and cerebral hemorrhage); endoplasmic reticulum associated protein 140 kDa; ESTs; ESTs; ESTs, Highly similar to hypothetical protein FLJ10350 [Homo sapiens] [H. sapiens]; ESTs, Highly similar to ITB1_HUMAN Integrin beta-1 precursor (Fibronectin receptor beta subunit) (CD29) (Integrin VLA-4 beta subunit) [H. sapiens]; ESTs, Weakly similar to hypothetical protein F1120489 [Homo sapiens] [H. sapiens]; ESTs, Weakly similar to T17346 hypothetical protein DKFZp586O1624.1—human (fragment) [H. sapiens]; ESTs, Weakly similar to T21371 hypothetical protein F25H8.3—Caenorhabditis elegans [C. elegans]; eukaryotic translation initiation factor 4A, isoform 1; heme oxygenase (decycling) 1; Hermansky-Pudlak syndrome 4; Homo sapiens cDNA FLJ34888 fis, clone NT2NE2017332; Homo sapiens cDNA FLJ39848 fis, clone SPLEN2014669; Homo sapiens mRNA full length insert cDNA clone EUROIMAGE 1977059; Homo sapiens, clone IMAGE:4845226, mRNA; hypothetical protein FLJ22329; hypothetical protein FLJ32205; hypothetical protein MGC4677; inhibin, beta B (activin AB beta polypeptide); insulin-like growth factor binding protein 5; junction plakoglobin; KIAA0620 protein; KIAA0943 protein; likely ortholog of rat vacuole membrane protein 1; Lysosomal-associated multispanning membrane protein-5; major histocompatibility complex, class I, B; major histocompatibility complex, class I, C; matrix Gla protein; matrix metalloproteinase 1 (interstitial collagenase); microtubule-associated protein 1 light chain 3 beta; nerve growth factor receptor (TNFR superfamily, member 16); ribosomal protein S9; ring finger protein 40; S100 calcium binding protein, beta (neural); sema domain, transmembrane domain (TM), and cytoplasmic domain, (semaphorin) 6B; SPARC-like 1 (mast9, hevin); tumor necrosis factor, alpha-induced protein 3; UDP-Gal:betaGlcNAc beta 1,4-galactosyltransferase, polypeptide 3; UDP-GlcNAc:betaGal beta-1,3-N-acetylglucosaminyltransferase 5; von Willebrand factor; v-akt murine thymoma vial oncogene homolog 2; cyclin-dependent kinase (cdc2-like) 10; ortholog mouse myocytic induction/differentiation originator; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16); insulin-like growth factor binding protein; leukemia inhibitory factor; protein tyrosine phosphatase, nonreceptor type I; and Homo sapiens, clone IMAGE:3908182, mRNA, partial cds. Expression of the at least one gene in the first brain tissue sample is compared to expression of the at least one gene in a second brain tissue sample which is normal. Increased expression of the at least one gene in the first brain tissue sample relative to the second tissue sample identifies the first brain tissue sample as likely to be neoplastic.

According to another embodiment of the invention a method is provided of treating a glioma. Cells of the glioma are contacted with an antibody. The antibody specifically binds to an extracellular epitope of a protein selected from the group consisting of plasmalemma associated protein; KIAA0726 gene product; osteonectin: laminin, alpha 5; collagen, type IV, alpha 1; insulin-like growth factor binding protein 7; Thy-1 cell surface antigen; dysferlin, limb girdle muscular dystrophy 2B; integrin, alpha 5; matrix metalloproteinase 9; Lutjheran blood group, integrink, alpha 10, collagen, type VI, alpha 2; glioma endothelial marker 1 precursor; translocase of inner mitochondrial membrane 17 homolog A; heparan sulfate proteoglycan 2; annexin A2; matrix metalloproteinase 10; G protein-coupled receptor; matrix metalloproteinase 14; solute carrier family 29, member 1; CD59 antigen p18-20; KIAA 1870 protein; plexin B2; lectin, glactoside-binding, soluble, 8; integrin beta 4 binding protein; acetyl LDL receptor; laminin, gamma 3; macrophage migration inhibitory factor; gap junction protein, alpha 1, 43 kD; aquaporin 1; protease, serine, 11; collagen, type IV, alpha 2; apolipoprotein D; plasminogen activator, urokinase; insulin-like growth factor binding protein 3; regulator of G-protein signaling 12; prosaposin; laminin, alpha 4; lectin, galactoside-binding, soluble, 3 binding protein; glycophorin C; endothelin receptor type B; biglycan; transmembrane 4 superfamilyh member 2; lysyl osidase-like 2; TEK tyrosine kinase, endothelial; insulin receptore; cell membrane glycoprotein, 110000M(r); jagged 1; plasmalemma vesicle associated protein; TEM13, Thy-1 cell surface antigen; coagulation factor II (thrombin) receptor-like 3; dysferlin, limb girdle muscular dystrophy 2B (autosomal recessive); sema domain, transmembrane domain (TM), and cytoplasmic domain, (semaphorin) 6B; integrin, alpha 5 (fibronectin receptor, alpha polypeptide); likely ortholog of rat vacuole membrane protein 1; nerve growth factor receptor (TNFR superfamily, member 16); degenerative spermatocyte homolog, lipid desaturase (Drosophila); TEM1, endosialin; heme oxygenase (decycling) 1; G protein-coupled receptor; C-type (calcium dependent, carbohydrate-recognition domain) lectin, superfamily member 9; matrix metalloproteinase 14 (membrane-inserted); solute carrier family 29 (nucleoside transporters), member 1; likely ortholog of mouse embryonic epithelial gene 1; major histocompatibility complex, class I, C; likely ortholog of mouse fibronectin type III repeat containing protein 1; sprouty homolog 4 (Drosophila); KIAA0620 protein; coagulation factor III (thromboplastin, tissue factor); aquaporin 1 (channel-forming integral protein, 28 kDa); major histocompatibility complex, class I, B; Lysosomal-associated multispanning membrane protein-5; endothelin receptor type B; insulin receptor; complement component 1, q subcomponent, receptor 1; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16) and complement component 1, q subcomponent, receptor 1. Immune destruction of cells of the glioma is thereby triggered.

According to still another embodiment of the invention a method is provided for identifying a test compound as a potential anti-cancer or anti-glioma drug. A test compound is contacted with a cell which expresses at least one gene selected from the group consisting of: signal sequence receptor, delta (translocon-associated protein delta); DC2 protein; KIAA0404 protein; symplekin; Huntingtin interacting protein I; plasmalemma vesicle associated protein; KIAA0726 gene product; latexin protein; transforming growth factor, beta 1; hypothetical protein FLJ22215; Rag C protein; hypothetical protein FLJ23471; N-myristoyltransferase 1; hypothetical protein dJ1181N3.1; ribosomal protein L27; secreted protein, acidic, cysteine-rich (osteonectin); Hs 111988; Hs 112238; laminin, alpha 5; protective protein for beta-galactosidase (galactosialidosis); Melanoma associated gene; Melanoma associated gene; E3 ubiquitin ligase SMURF1; collagen, type IV, alpha 1; collagen, type IV, alpha 1; collagen, type IV, alpha 1; insulin-like growth factor binding protein 7; gene predicted from cDNA with a complete coding sequence; Thy-1 cell surface antigen; Hs 127824; GTP binding protein 2; Homo sapiens mRNA; cDNA DKFZp586D0918 (from clone DKFZp586D0918); cutaneous T-cell lymphoma-associated tumor antigen se20-4; differentially expressed nucleolar TGF-beta1 target protein (DENTT); dysferlin, limb girdle muscular dystrophy 2B (autosomal recessive); smoothelin; integrin, alpha 5 (fibronectin receptor, alpha polypeptide); putative translation initiation factor; retinoic acid induced 14; matrix metalloproteinase 9 (gelatinase B, 92 kD gelatinase, 92 kD type IV collagenase); Lutheran blood group (Auberger b antigen included); stanniocalcin 2; nuclear factor (erythroid-derived 2)-like 2; protein tyrosine phosphatase, non-receptor type 1; integrin, alpha 10; collagen, type VI, alpha 2; chromosome 21 open reading frame 25; CDC37 (cell division cycle 37, S. cerevisiae, homolog); Hs 16450; Rho guanine nucleotide exchange factor (GEF) 7; creatine kinase, brain; hypothetical protein FLJ10297; hypothetical protein FLJ10350; TNF-induced protein; tumor necrosis factor receptor superfamily, member 12 (translocating chain-association membrane protein); cofilin 1 (non-muscle); splicing factor proline/glutamine rich (polypyrimidine tract-binding protein-associated); splicing factor proline/glutamine rich (polypyrimidine tract-binding protein-associated); v-ets avian erythroblastosis virus E26 oncogene homolog 1; protease, cysteine, 1 (legumain); ribosomal protein L13; chromosome 22 open reading frame 5; zinc finger protein 144 (MeI-18); degenerative spermatocyte (homolog Drosophila; lipid desaturase); eukaryotic translation initiation factor 2C, 2; mitochondrial ribosomal protein L45; prostate tumor over expressed gene 1; NADH dehydrogenase (ubiquinone) 1 alpha subcomplex, 7 (14.5 kD, B14.5a); glioma endothelial marker 1 precursor; NS1-binding protein; ribosomal protein L38; tuftelin-interacting protein; HLA class II region expressed gene KE2; translocase of inner mitochondrial membrane 17 homolog A (yeast); sudD (suppressor of bimD6, Aspergillus nidulans) homolog; heparan sulfate proteoglycan 2 (perlecan); SEC24 (S. cerevisiae) related gene family, member A; NADH dehydrogenase (ubiquinone) Fe—S protein 7 (20 kD) (NADH-coenzyme Q reductase); DNA segment on chromosome X and Y (unique) 155 expressed sequence; annexin A2; Homo sapiens clone 24670 mRNA sequence; hypothetical protein; matrix metalloproteinase 10 (stromelysin 2); KIAA1049 protein; G protein-coupled receptor; hypothetical protein FLJ20401; matrix metalloproteinase 14 (membrane-inserted); KIAA0470 gene product; solute carrier family 29 (nucleoside transporters), member 1; stanniocalcin 1; stanniocalcin 1; stanniocalcin 1; tumor suppressor deleted in oral cancer-related 1; tumor suppressor deleted in oral cancer-related 1; apolipoprotein C—I; glutathione peroxidase 4 (phospholipid hydroperoxidase); Hs 272106; transcription factor binding to IGHM enhancer 3; hypothetical protein DKFZp762A227; hypothetical protein FLJ22362; CD59 antigen p 18-20 (antigen identified by monoclonal antibodies 16.3A5, EJ16, EJ30, EL32 and G344); PRO0628 protein; melanoma-associated antigen recognised by cytotoxic T lymphocytes; LOC88745; Homo sapiens beta-1,3-galactosyltransferase-6 (B3GALT6) mRNA, complete cds; sprouty (Drosophila) homolog 4; sprouty (Drosophila) homolog 4; Homo sapiens mRNA; cDNA DKFZp434E1515 (from clone DKFZp434E1515); coactosin-like protein; hypothetical protein FLJ21865; Hs296234; KIAA0685 gene product; hypothetical protein FLJ10980; ribosomal protein L10; ribosomal protein S19; Hs 299251; Huntingtin interacting protein K; Homo sapiens mRNA full length insert cDNA clone EUROIMAGE 50374; Hs 311780; Hs 212191; v-akt murine thymoma viral oncogene homolog 2; Hs 328774; transducin-like enhancer of split 2, homolog of Drosophila E(sp1); KIAA1870 protein; ribosomal protein L10a; peptidylprolyl isomerase A (cyclophilin A); Hs 344224; hypothetical protein F1123239; hypothetical protein DKFZp761H221; KIAA1887 protein; Homo sapiens mRNA full length insert cDNA clone EUROIMAGE 701679; Homo sapiens cDNA F1130634 fis, clone CTONG2002453; Homo sapiens cDNA FLJ32203 fis, clone PLACE6003038, weakly similar to ZINC FINGER PROTEIN 84; Homo sapiens mRNA full length insert cDNA clone EUROIMAGE 1035904; hypothetical protein L0057333; myosin ID; plexin B2; lectin, galactoside-binding, soluble, 8 (galectin 8); double ring-finger protein, Dorfin; DKFZP434B168 protein; LIM domain binding 2; integrin beta 4 binding protein; synaptopodin; Hs 54828; insulin induced gene 1; acetyl LDL receptor; SREC; excision repair cross-complementing rodent repair deficiency, complementation group 1 (includes overlapping antisense sequence); hypothetical protein F1122329; schwannomin-interacting protein 1; PTEN induced putative kinase 1; myosin X; Homo sapiens cDNA FLJ32424 fis, clone SKMUS2000954, moderately similar to Homo sapiens F-box protein Fbx25 (FBX25) 97; golgi phosphoprotein 1; splicing factor, arginine/serine-rich 6; laminin, gamma 3; cysteine-rich protein 2; U6 snRNA-associated Sm-like protein LSm7; hypothetical protein FLJ10707; Homo sapiens, Similar to RIKEN cDNA 2310012N15 gene, clone IMAGE:3342825, mRNA, partial cds; macrophage migration inhibitory factor (glycosylation-inhibiting factor); ubiquinol-cytochrome c reductase hinge protein; gap junction protein, alpha 1, 43 kD (connexin 43); dihydropyrimidinase-like 3; aquaporin 1 (channel-forming integral protein, 28 kD); protein expressed in thyroid; macrophage myristoylated alanine-rich C kinase substrate; procollagen-lysine, 2-oxoglutarate 5-dioxygenase (lysine hydroxylase, Ehlers-Danlos syndrome type VI); protease, serine, 11 (IGF binding); 24-dehydrocholesterol reductase; collagen, type IV, alpha 2; profilin 1; apolipoprotein D; hyaluronoglucosaminidase 2; hypothetical protein FLJ22678; quiescin Q6; ras homolog gene family, member A; ras homolog gene family, member A; plasminogen activator, urokinase; insulin-like growth factor binding protein 3; uridine phosphorylase; KIAA0638 protein; B7 homolog 3; lamin A/C; lamin A/C; lamin A/C; regulator of G-protein signalling 12; proteasome (prosome, macropain) 26S subunit, non-ATPase, 8; Homo sapiens, Similar to RIKEN cDNA 5730528L13 gene, clone MGC:17337 IMAGE:4213591, mRNA, complete cds; prosaposin (variant Gaucher disease and variant metachromatic leukodystrophy); laminin, alpha 4; transcription elongation factor A (SII), 1; lectin, galactoside-binding, soluble, 3 binding protein; ribosomal protein S16; glycophorin C (Gerbich blood group); endothelin receptor type B; serine (or cysteine) proteinase inhibitor, Glade E (nexin, plasminogen activator inhibitor type 1), member 1; biglycan; small nuclear ribonucleoprotein polypeptide B″; transmembrane 4 superfamily member 2; TAF11 RNA polymerase II, TATA box binding protein (TBP)-associated factor, 28 kD; lysyl oxidase-like 2; SRY (sex determining region Y)-box 4; SOX4 SRY (sex determining region Y)-box 4; SRY (sex determining region Y)-box 4; actin related protein 2/3 complex, subunit 2 (34 kD); Homo sapiens cDNA: FLJ23507 fis, clone LNG03128; hypothetical protein FLJ12442; Fas (TNFRSF6)-associated via death domain; mitogen-activated protein kinase kinase kinase 11; TEK tyrosine kinase, endothelial (venous malformations, multiple cutaneous and mucosal); insulin receptor; cell membrane glycoprotein, 110000M(r) (surface antigen); Homo sapiens cDNA FLJ11863 fis, clone HEMBA1006926; jagged 1 (Alagille syndrome); KIAA0304 gene product; pre-B-cell leukemia transcription factor 2; Homo sapiens cDNA FLJ31238 fis, clone KIDNE2004864; p53-induced protein; complement component 1, q subcomponent, receptor 1; complement component 1, q subcomponent, receptor 1; apolipoprotein E; chemokine (C—C motif) ligand 3; coagulation factor II (thrombin) receptor-like 3; coagulation factor III (thromboplastin, tissue factor); collagen, type I, alpha 1; collagen, type III, alpha 1 (Ehlers-Danlos syndrome type IV, autosomal dominant); C-type (calcium dependent, carbohydrate-recognition domain) lectin, superfamily member 9; cystatin C (amyloid angiopathy and cerebral hemorrhage); endoplasmic reticulum associated protein 140 kDa; ESTs; ESTs; ESTs, Highly similar to hypothetical protein FLJ10350 [Homo sapiens] [H. sapiens]; ESTs, Highly similar to ITB1_HUMAN Integrin beta-1 precursor (Fibronectin receptor beta subunit) (CD29) (Integrin VLA-4 beta subunit) [H. sapiens]; ESTs, Weakly similar to hypothetical protein FLJ20489 [Homo sapiens] [H. sapiens]; ESTs, Weakly similar to T17346 hypothetical protein DKFZp586O1624.1—human (fragment) [H. sapiens]; ESTs, Weakly similar to T21371 hypothetical protein F25H8.3—Caenorhabditis elegans [C. elegans]; eukaryotic translation initiation factor 4A, isoform 1; heme oxygenase (decycling) 1; Hermansky-Pudlak syndrome 4; Homo sapiens cDNA FLJ34888 fis, clone NT2NE2017332; Homo sapiens cDNA FLJ39848 fis, clone SPLEN2014669; Homo sapiens mRNA full length insert cDNA clone EUROIMAGE 1977059; Homo sapiens, clone IMAGE:4845226, mRNA; hypothetical protein FLJ22329; hypothetical protein FLJ32205; hypothetical protein MGC4677; inhibin, beta B (activin AB beta polypeptide); insulin-like growth factor binding protein 5; junction plakoglobin; KIAA0620 protein; KIAA0943 protein; likely ortholog of rat vacuole membrane protein 1; Lysosomal-associated multispanning membrane protein-5; major histocompatibility complex, class I, B; major histocompatibility complex, class I, C; matrix Gla protein; matrix metalloproteinase 1 (interstitial collagenase); microtubule-associated protein 1 light chain 3 beta; nerve growth factor receptor (TNFR superfamily, member 16); ribosomal protein S9; ring finger protein 40; S100 calcium binding protein, beta (neural); sema domain, transmembrane domain (TM), and cytoplasmic domain, (semaphorin) 6B; SPARC-like 1 (mast9, hevin); tumor necrosis factor, alpha-induced protein 3; UDP-Gal:betaGlcNAc beta 1,4-galactosyltransferase, polypeptide 3; UDP-GlcNAc:betaGal beta-1,3-N-acetylglucosaminyltransferase 5; von Willebrand factor; v-akt murine thymoma vial oncogene homolog 2; cyclin-dependent kinase (cdc2-like) 10; ortholog mouse myocytic induction/differentiation originator; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16); insulin-like growth factor binding protein; leukemia inhibitory factor; protein tyrosine phosphatase, nonreceptor type I; and Homo sapiens, clone IMAGE:3908182, mRNA, partial cds. An expression product of the at least one gene is monitored. The test compound is identified as a potential anti-cancer drug if it decreases the expression of the at least one gene.

According to yet another embodiment of the invention a method is provided to aid in diagnosing glioma. An mRNA of at least one gene in a first brain tissue sample suspected of being neoplastic is detected. The at least one gene is identified by a tag selected from the group consisting of SEQ ID NO: 1-32. Expression of the at least one gene in the first brain tissue sample is compared to expression of the at least one gene in a second brain tissue sample which is normal. If increased expression of the at least one gene in the first brain tissue sample relative to the second tissue sample if found, the first brain tissue sample is identified as likely to be neoplastic.

Another embodiment of the invention is a method of identifying a test compound as a potential anti-cancer or anti-glioma drug. A test compound is contacted with a cell. The cell expresses an mRNA of at least one gene identified by a tag selected from the group consisting of SEQ ID NO: 1-32. An mRNA of the at least one gene is monitored. The test compound is identified as a potential anti-cancer drug if it decreases the expression of at least one gene.

Still another embodiment of the invention is a method to induce an immune response to glioma. A protein or nucleic acid encoding a protein is administered to a mammal, preferably a human. The protein is selected from the group consisting of: signal sequence receptor, delta (translocon-associated protein delta); DC2 protein; KIAA0404 protein; symplekin; Huntingtin interacting protein I; plasmalemma vesicle associated protein; KIAA0726 gene product; latexin protein; transforming growth factor, beta 1; hypothetical protein FLJ22215; Rag C protein; hypothetical protein FLJ23471; N-myristoyltransferase 1; hypothetical protein dJ1181N3.1; ribosomal protein L27; secreted protein, acidic, cysteine-rich (osteonectin); Hs 111988; Hs 112238; laminin, alpha 5; protective protein for beta-galactosidase (galactosialidosis); Melanoma associated gene; Melanoma associated gene; E3 ubiquitin ligase SMURF1; collagen, type IV, alpha 1; collagen, type IV, alpha 1; collagen, type IV, alpha 1; insulin-like growth factor binding protein 7; gene predicted from cDNA with a complete coding sequence; Thy-1 cell surface antigen; Hs 127824; GTP binding protein 2; Homo sapiens mRNA; cDNA DKFZp586D0918 (from clone DKFZp586D0918); cutaneous T-cell lymphoma-associated tumor antigen se20-4; differentially expressed nucleolar TGF-beta1 target protein (DENTT); dysferlin, limb girdle muscular dystrophy 2B (autosomal recessive); smoothelin; integrin, alpha 5 (fibronectin receptor, alpha polypeptide); putative translation initiation factor; retinoic acid induced 14; matrix metalloproteinase 9 (gelatinase B, 92 kD gelatinase, 92 kD type IV collagenase); Lutheran blood group (Auberger b antigen included); stanniocalcin 2; nuclear factor (erythroid-derived 2)-like 2; protein tyrosine phosphatase, non-receptor type 1; integrin, alpha 10; collagen, type VI, alpha 2; chromosome 21 open reading frame 25; CDC37 (cell division cycle 37, S. cerevisiae, homolog); Hs 16450; Rho guanine nucleotide exchange factor (GEF) 7; creatine kinase, brain; hypothetical protein FLJ10297; hypothetical protein FLJ10350; TNF-induced protein; tumor necrosis factor receptor superfamily, member 12 (translocating chain-association membrane protein); cofilin 1 (non-muscle); splicing factor proline/glutamine rich (polypyrimidine tract-binding protein-associated); splicing factor proline/glutamine rich (polypyrimidine tract-binding protein-associated); v-ets avian erythroblastosis virus E26 oncogene homolog 1; protease, cysteine, 1 (legumain); ribosomal protein L13; chromosome 22 open reading frame 5; zinc finger protein 144 (Mel-18); degenerative spermatocyte (homolog Drosophila; lipid desaturase); eukaryotic translation initiation factor 2C, 2; mitochondrial ribosomal protein L45; prostate tumor over expressed gene 1; NADH dehydrogenase (ubiquinone) 1 alpha subcomplex, 7 (14.5 kD, B14.5a); glioma endothelial marker 1 precursor; NS1-binding protein; ribosomal protein L38; tuftelin-interacting protein; HLA class II region expressed gene KE2; translocase of inner mitochondrial membrane 17 homolog A (yeast); sudD (suppressor of bimD6, Aspergillus nidulans) homolog; heparan sulfate proteoglycan 2 (perlecan); SEC24 (S. cerevisiae) related gene family, member A; NADH dehydrogenase (ubiquinone) Fe—S protein 7 (20 kD) (NADH-coenzyme Q reductase); DNA segment on chromosome X and Y (unique) 155 expressed sequence; annexin A2; Homo sapiens clone 24670 mRNA sequence; hypothetical protein; matrix metalloproteinase 10 (stromelysin 2); KIAA1049 protein; G protein-coupled receptor; hypothetical protein FLJ20401; matrix metalloproteinase 14 (membrane-inserted); KIAA0470 gene product; solute carrier family 29 (nucleoside transporters), member 1; stanniocalcin 1; stanniocalcin 1; stanniocalcin 1; tumor suppressor deleted in oral cancer-related 1; tumor suppressor deleted in oral cancer-related 1; apolipoprotein C—I; glutathione peroxidase 4 (phospholipid hydroperoxidase); Hs 272106; transcription factor binding to IGHM enhancer 3; hypothetical protein DKFZp762A227; hypothetical protein FLJ22362; CD59 antigen p18-20 (antigen identified by monoclonal antibodies 163A5, EJ16, EJ30, EL32 and G344); PRO0628 protein; melanoma-associated antigen recognised by cytotoxic T lymphocytes; LOC88745; Homo sapiens beta-1,3-galactosyltransferase-6 (B3GALT6) mRNA, complete cds; sprouty (Drosophila) homolog 4; sprouty (Drosophila) homolog 4; Homo sapiens mRNA; cDNA DKFZp434E1515 (from clone DKFZp434E1515); coactosin-like protein; hypothetical protein FLJ21865; Hs296234; KIAA0685 gene product; hypothetical protein FLJ10980; ribosomal protein L10; ribosomal protein S19; Hs 299251; Huntingtin interacting protein K; Homo sapiens mRNA full length insert cDNA clone EUROIMAGE 50374; Hs 311780; Hs 212191; v-akt murine thymoma viral oncogene homolog 2; Hs 328774; transducin-like enhancer of split 2, homolog of Drosophila E(sp1); KIAA1870 protein; ribosomal protein L10a; peptidylprolyl isomerase A (cyclophilin A); Hs 344224; hypothetical protein FLJ23239; hypothetical protein DKFZp761H221; KIAA1887 protein; Homo sapiens mRNA full length insert cDNA clone EUROIMAGE 701679; Homo sapiens cDNA FLJ30634 fis, clone CTONG2002453; Homo sapiens cDNA FLJ32203 fis, clone PLACE6003038, weakly similar to ZINC FINGER PROTEIN 84; Homo sapiens mRNA full length insert cDNA clone EUROIMAGE 1035904; hypothetical protein L0057333; myosin ID; plexin B2; lectin, galactoside-binding, soluble, 8 (galectin 8); double ring-finger protein, Dorfin; DKFZP434B 168 protein; LIM domain binding 2; integrin beta 4 binding protein; synaptopodin; Hs 54828; insulin induced gene 1; acetyl LDL receptor; SREC; excision repair cross-complementing rodent repair deficiency, complementation group 1 (includes overlapping antisense sequence); hypothetical protein FLJ22329; schwannomin-interacting protein 1; PTEN induced putative kinase 1; myosin X; Homo sapiens cDNA FLJ32424 fis, clone SKMUS2000954, moderately similar to Homo sapiens F-box protein Fbx25 (FBX25) 97; golgi phosphoprotein 1; splicing factor, arginine/serine-rich 6; laminin, gamma 3; cysteine-rich protein 2; U6 snRNA-associated Sm-like protein LSm7 hypothetical protein FLJ10707; Homo sapiens, Similar to RIKEN cDNA 2310012N15 gene, clone IMAGE:3342825, mRNA, partial cds; macrophage migration inhibitory factor (glycosylation-inhibiting factor); ubiquinol-cytochrome c reductase hinge protein; gap junction protein, alpha 1, 43 kD (connexin 43); dihydropyrimidinase-like 3; aquaporin 1 (channel-forming integral protein, 28 kD); protein expressed in thyroid; macrophage myristoylated alanine-rich C kinase substrate; procollagen-lysine, 2-oxoglutarate 5-dioxygenase (lysine hydroxylase, Ehlers-Danlos syndrome type VI); protease, serine, 11 (IGF binding); 24-dehydrocholesterol reductase; collagen, type IV, alpha 2; profilin 1; apolipoprotein D; hyaluronoglucosaminidase 2; hypothetical protein FLJ22678; quiescin Q6; ras homolog gene family, member A; ras homolog gene family, member A; plasminogen activator, urokinase; insulin-like growth factor binding protein 3; uridine phosphorylase; KIAA0638 protein; B7 homolog 3; lamin A/C; lamin A/C; lamin A/C; regulator of G-protein signalling 12; proteasome (prosome, macropain) 26S subunit, non-ATPase, 8; Homo sapiens, Similar to RIKEN cDNA 5730528L13 gene, clone MGC:17337 IMAGE:4213591, mRNA, complete cds; prosaposin (variant Gaucher disease and variant metachromatic leukodystrophy); laminin, alpha 4; transcription elongation factor A (SII), 1; lectin, galactoside-binding, soluble, 3 binding protein; ribosomal protein S16; glycophorin C (Gerbich blood group); endothelin receptor type B; serine (or cysteine) proteinase inhibitor, Glade E (nexin, plasminogen activator inhibitor type 1), member 1; biglycan; small nuclear ribonucleoprotein polypeptide B″; transmembrane 4 superfamily member 2; TAF11 RNA polymerase II, TATA box binding protein (TBP)-associated factor, 28 kD; lysyl oxidase-like 2; SRY (sex determining region Y)-box 4; SOX4 SRY (sex determining region Y)-box 4; SRY (sex determining region Y)-box 4; actin related protein 2/3 complex, subunit 2 (34 kD); Homo sapiens cDNA: FLJ23507 fis, clone LNG03128; hypothetical protein FLJ12442; Fas (TNFRSF6)-associated via death domain; mitogen-activated protein kinase kinase kinase 11; TEK tyrosine kinase, endothelial (venous malformations, multiple cutaneous and mucosal); insulin receptor; cell membrane glycoprotein, 110000M(r) (surface antigen); Homo sapiens cDNA FLJ11863 fis, clone HEMBA1006926; jagged 1 (Alagille syndrome); KIAA0304 gene product; pre-B-cell leukemia transcription factor 2; Homo sapiens cDNA FLJ31238 fis, clone KIDNE2004864; p53-induced protein; complement component 1, q subcomponent, receptor 1; complement component 1, q subcomponent, receptor 1; apolipoprotein E; chemokine (C—C motif) ligand 3; coagulation factor II (thrombin) receptor-like 3; coagulation factor III (thromboplastin, tissue factor); collagen, type I, alpha 1; collagen, type III, alpha 1 (Ehlers-Danlos syndrome type IV, autosomal dominant); C-type (calcium dependent, carbohydrate-recognition domain) lectin, superfamily member 9; cystatin C (amyloid angiopathy and cerebral hemorrhage); endoplasmic reticulum associated protein 140 kDa; ESTs; ESTs; ESTs, Highly similar to hypothetical protein FLJ10350 [Homo sapiens] [H. sapiens]; ESTs, Highly similar to ITB1_HUMAN Integrin beta-1 precursor (Fibronectin receptor beta subunit) (CD29) (Integrin VLA-4 beta subunit) [H. sapiens]; ESTs, Weakly similar to hypothetical protein FLJ20489 [Homo sapiens] [H. sapiens]; ESTs, Weakly similar to T17346 hypothetical protein DKFZp586O1624.1—human (fragment) [H. sapiens]; ESTs, Weakly similar to T21371 hypothetical protein F25H8.3—Caenorhabditis elegans [C. elegans]; eukaryotic translation initiation factor 4A, isoform 1; heme oxygenase (decycling) 1; Hermansky-Pudlak syndrome 4; Homo sapiens cDNA FLJ34888 fis, clone NT2NE2017332; Homo sapiens cDNA FLJ39848 fis, clone SPLEN2014669; Homo sapiens mRNA full length insert cDNA clone EUROIMAGE 1977059; Homo sapiens, clone IMAGE:4845226, mRNA; hypothetical protein FLJ22329; hypothetical protein FLJ32205; hypothetical protein MGC4677; inhibin, beta B (activin AB beta polypeptide); insulin-like growth factor binding protein 5; junction plakoglobin; KIAA0620 protein; KIAA0943 protein; likely ortholog of rat vacuole membrane protein 1; Lysosomal-associated multispanning membrane protein-5; major histocompatibility complex, class 1, B; major histocompatibility complex, class I, C; matrix Gla protein; matrix metalloproteinase 1 (interstitial collagenase); microtubule-associated protein 1 light chain 3 beta; nerve growth factor receptor (TNFR superfamily, member 16); ribosomal protein S9; ring finger protein 40; S100 calcium binding protein, beta (neural); sema domain, transmembrane domain (TM), and cytoplasmic domain, (semaphorin) 6B; SPARC-like 1 (mast9, hevin); tumor necrosis factor, alpha-induced protein 3; UDP-Gal:betaGlcNAc beta 1,4-galactosyltransferase, polypeptide 3; UDP-GlcNAc:betaGal beta-1,3-N-acetylglucosaminyltransferase 5; von Willebrand factor; v-akt murine thymoma vial oncogene homolog 2; cyclin-dependent kinase (cdc2-like) 10; ortholog mouse myocytic induction/differentiation originator; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16); insulin-like growth factor binding protein; leukemia inhibitory factor; protein tyrosine phosphatase, nonreceptor type I; and Homo sapiens, clone IMAGE:3908182, mRNA, partial cds. An immune response to the protein is thereby induced.

The present invention thus provides the art with methods of diagnosing and treating gliomas and other brain tumors.

### DETAILED DESCRIPTION OF THE INVENTION

Using SAGE (Serial Analysis of Gene Expression) profiling, this study was able to identify previously unrecognized, angiogenesis-specific markers that discriminate between non-proliferative and pathologic endothelial cells. We identified 255 human genes that were expressed at significantly higher levels in brain tumor endothelium than in normal brain endothelium. See Table 1. We have named these markers GEMs (glioma endothelial markers). Any of the GEMs disclosed in any of the tables can be used in the methods of the present invention, according to the discretion of the skilled artisan.

ECs represent only a minor fraction of the total cells within normal or tumor tissues, and only those EC transcripts expressed at the highest levels would be expected to be represented in libraries constructed from unfractionated tissues. The genes described in the current study should therefore provide a valuable resource for basic and clinical studies of human brain angiogenesis in the future. Genes which have been identified as expressed more in glioma endothelial cells than in normal brain endothelial cells (GEMs) include those which correspond to tags shown in SEQ ID NOS: 1-32. The tags correspond to the segment of the cDNA that is 3′ of the 3′ most restriction endonuclease site for the restriction enzyme NlaIII which was used as the anchoring enzyme. The tag shown is the same strand as the mRNA. Other such genes are listed in Tables 1 and 2.

TABLE 1

| StdTag | SEQ | LongTag | SEQ ID | Function |
|---|---|---|---|---|
| AAACCATTCT | 1 | AAACCATTCTCCTCCGC | 256 | |
| AAGGCAGGGA | 2 | AAGGCAGGGAGGGAGGG | 257 | |
| ACACAGCAAG | 3 | ACACAGCAAGACGAGAA | 258 | |
| AGCTGGAGTC | 4 | AGCTGGAGTCCTAGGCA | 259 | |
| AGCTGGCACC | 5 | AGCTGGCACCAGAGCCC | 260 | |
| ATAAATGAGG | 6 | ATAAATGAGGTAAGGTC | 261 | |
| CAAGCACCCC | 7 | CAAGCACCCCCGTTCCA | 262 | |
| CACTACCCAC | 8 | CACTACCCACCAGACGC | 263 | |
| CACTACTCAC | 9 | CACTACTCACCAGGCGC | 264 | |
| CCCACCTCCA | 10 | CCCACCTCCAGTCCAGC | 265 | |
| CCCGCCTCTT | 11 | CCCGCCTCTTCACGGGC | 266 | |
| CCTCAGATGT | 12 | CCTCAGATGTTTGAAAA | 267 | |
| CGCTACTCAC | 13 | CGCTACTCACCAGACGC | 268 | |
| CTAAGACCTC | 14 | CTAAGACCTCACCAGTC | 269 | |
| CTAAGACTTC | 15 | CTAAGACTTCACCGGTC | 270 | |
| GAGTGGGTGC | 16 | GAGTGGGTGCAGCCTCC | 271 | |
| GGGACAGCTG | 17 | GGGACAGCTGTCTGTGG | 272 | |
| GGGTTGGCTT | 18 | GGGTTGGCTTGAAACCA | 273 | |
| GTAAGTGTAC | 19 | GTAAGTGTACTGGAAGT | 274 | |
| GTAAGTGTAC | 20 | GTAAGTGTACTGGTAAG | 275 | |
| GTAGGGGTAA | 21 | GTAGGGGTAAAAGGAGG | 276 | |
| TAACCACTGC | 22 | TAACCACTGCACTTTCC | 277 | |
| TACTGCTCGG | 23 | TACTGCTCGGAGGTCGG | 278 | |
| TCAGGCTGAA | 24 | TCAGGCTGAAGTCAGGC | 279 | |
| TCCATACACC | 25 | TCCATACACCTATCCCC | 280 | |
| TCCTTTTAAA | 26 | TCCTTTTAAAACAAAAC | 281 | |
| TGATTAAGGT | 27 | TGATTAAGGTCGGCGCT | 282 | |
| TGGTATCACA | 28 | TGGTATCACACAAGGGG | 283 | |
| TGGTGTATGC | 29 | TGGTGTATGCATCGGGG | 284 | |
| TGTCACTGGG | 30 | TGTCACTGGGCAGGCGG | 285 | |
| TGTGGGAGGC | 31 | TGTGGGAGGCTGATGGG | 286 | |
| TTTAACGGCC | 32 | TTTAACGGCCGCGGTAC | 287 | |
| GCTCTCTATG | 33 | GCTCTCTATGCTGACGT | 288 | signal sequence receptor, delta (translocon-associated |
| | | | | protein delta) |
| AGAATGAAAC | 34 | AGAATGAAACTGCCGGG | 289 | DC2 protein |
| AAGTGGAATA | 35 | AAGTGGAATAAACTGCC | 290 | KIAA0404 protein |
| GATGACGACT | 36 | GATGACGACTCGGGGCT | 291 | symplekin; Huntingtin interacting protein I |
| CCCTTTCACA | 37 | CCCTTTCACACACACTT | 292 | plasmalemma vesicle associated protein |
| TCCTGGGGCA | 38 | TCCTGGGGCAGGGGCGG | 293 | KIAA0726 gene product |
| TCTATTGATG | 39 | TCTATTGATGTGTATGC | 294 | latexin protein |
| GGGGCTGTAT | 40 | GGGGCTGTATTTAAGGA | 295 | transforming growth factor, beta 1 |
| CCCAGGACAC | 41 | CCCAGGACACCAGCTGG | 296 | hypothetical protein FLJ22215 |
| GGAGCTGCTG | 42 | GGAGCTGCTGCTTGTGG | 297 | Rag C protein |
| TGGACAGCAG | 43 | TGGACAGCAGGGACCTG | 298 | hypothetical protein FLJ23471 |
| TCTGGGAACA | 44 | TCTGGGAACAGGGACGG | 299 | N-myristoyltransferase 1 |
| CCTGTGTATG | 45 | CCTGTGTATGTGTGTAA | 300 | hypothetical protein dJ1181N3.1 |
| GGCAAGAAGA | 46 | GGCAAGAAGAAGATCGC | 301 | ribosomal protein L27 |
| AAATGCTTGG | 47 | AAATGCTTGGAGGTGAA | 302 | secreted protein, acidic, cysteine-rich (osteonectin) |
| CTAAAAACCT | 48 | CTAAAAACCTTATGACA | 303 | secreted protein, acidic, cysteine-rich (osteonectin) |
| GAGCATTGCA | 49 | GAGCATTGCACCACCCG | 304 | secreted protein, acidic, cysteine-rich (osteonectin) |
| GGTGGACACG | 50 | GGTGGACACGGATCTGC | 305 | secreted protein, acidic, cysteine-rich (osteonectin) |
| GCTCCTGAGC | 51 | GCTCCTGAGCCCCGGCC | 306 | ESTs, Weakly similar to I65992 gene MLL protein |
| | | | | [H. sapiens] |
| AAGAAGTGGA | 52 | AAGAAGTGGAGATTGTC | 307 | ESTs |
| TGGGAAGTGG | 53 | TGGGAAGTGGGCTCCTT | 308 | maternally expressed 3 |
| ACTCGCTCTG | 54 | ACTCGCTCTGTGGAGGT | 309 | laminin, alpha 5 |
| TTTCAGGGGA | 55 | TTTCAGGGGAGGGGGAA | 310 | protective protein for beta-galactosidase |
| | | | | (galactosialidosis) |
| ACAACGTCCA | 56 | ACAACGTCCAGCTGGTG | 311 | Melanoma associated gene |
| GTCTCAGTGC | 57 | GTCTCAGTGCTGAGGCG | 312 | Melanoma associated gene |
| CCCCCTGCCC | 58 | CCCCCTGCCCCTCTGCC | 313 | E3 ubiquitin ligase SMURF1 |
| AGAAACCACG | 59 | AGAAACCACGGAAATGG | 314 | collagen, type IV, alpha 1 |
| GACCGCAGGA | 60 | GACCGCAGGAGGGCAGA | 315 | collagen, type IV, alpha 1 |
| GTGCTACTTC | 61 | GTGCTACTTCTTCTTCT | 316 | collagen, type IV, alpha 1 |
| GATAACTACA | 62 | GATAACTACATTACCTG | 317 | insulin-like growth factor binding protein 7 |
| TGGCTGTGAC | 63 | TGGCTGTGACTGTGACT | 318 | gene predicted from cDNA with a complete coding |
| | | | | sequence |
| GAGTGAGACC | 64 | GAGTGAGACCCAGGAGC | 319 | Thy-1 cell surface antigen |
| GAGTGGCTAC | 65 | GAGTGGCTACCCGCCGC | 320 | ESTs, Weakly similar to T28770 hypothetical protein |
| | | | | W03D2.1-Caenorhabditis elegans |
| GACTCAGGGA | 66 | GACTCAGGGATTTGTTG | 321 | GTP binding protein 2 |
| GTTATATGCC | 67 | GTTATATGCCCGGGAGA | 322 | Homo sapiens mRNA; cDNA DKFZp586D0918 (from clone |
| GAGGCGCTGC | 68 | GAGGCGCTGCTGCCACC | 323 | cutaneous T-cell lymphoma-associated tumor antigen |
| | | | | se20-4; differentially expressed nucleolar TGF-beta1 |
| | | | | target protein (DENTT) |
| GAGCTCTGAG | 69 | GAGCTCTGAGATCACCC | 324 | dysferlin, limb girdle muscular dystrophy 2B |
| | | | | (autosomal recessive) |
| GCCAGCCAGT | 70 | GCCAGCCAGTGGCAAGC | 325 | Smoothelin |
| ATGGCAACAG | 71 | ATGGCAACAGATCTGGA | 326 | integrin, alpha 5 (fibronectin receptor, alpha |
| | | | | polypeptide) |
| AAGGAGTTAC | 72 | AAGGAGTTACACTAGTC | 327 | putative translation initiation factor |
| TCCCACAAGG | 73 | TCCCACAAGGCTGCTTG | 328 | retinoic acid induced 14 |
| TAAATCCCCA | 74 | TAAATCCCCACTGGGAC | 329 | matrix metalloproteinase 9 (gelatinase B, 92 kD |
| | | | | gelatinase, 92 kD type IV collagenase) |
| CCCGCCCCCG | 75 | CCCGCCCCCGCCTTCCC | 330 | Lutheran blood group (Auberger b antigen included) |
| CCCGAGGCAG | 76 | CCCGAGGCAGAGTCGGG | 331 | stanniocalcin 2 |
| CTACGTGATG | 77 | CTACGTGATGAAGATGG | 332 | nuclear factor (erythroid-derived 2)-like 2 |
| ATGGGTTTGC | 78 | ATGGGTTTGCATTTTAG | 333 | protein tyrosine phosphatase, non-receptor type 1 |
| GGCATTGTCT | 79 | GGCATTGTCTCTGTTTC | 334 | integrin, alpha 10 |
| GTGCTAAGCG | 80 | GTGCTAAGCGGGCCCGG | 335 | collagen, type VI, alpha 2 |
| ACCGTTTGCA | 81 | ACCGTTTGCATTCGAAA | 336 | chromosome 21 open reading frame 25 |
| CAGCGCTGCA | 82 | CAGCGCTGCATTGACTC | 337 | CDC37 (cell division cycle 37, S. cerevisiae, |
| | | | | homolog) |
| GAAGACACTT | 83 | GAAGACACTTGGTTTGA | 338 | ESTs |
| CGCTGGGCGT | 84 | CGCTGGGCGTCTGGGAC | 339 | Rho guanine nucleotide exchange factor (GEF) 7 |
| CACCCCTGAT | 85 | CACCCCTGATGTTCGCC | 340 | creatine kinase, brain |
| GCCCCCCTGC | 86 | GCCCCCCTGCCCCGTGC | 341 | hypothetical protein FLJ10297 |
| CCCCCTGCCC | 87 | CCCCCTGCCCTCGCCTG | 342 | hypothetical protein FLJ10350 |
| AGCATAAAAA | 88 | AGCATAAAAATGCGTGC | 343 | TNF-induced protein |
| GGGCTGGACG | 89 | GGGCTGGACGGCTGCGT | 344 | tumor necrosis factor receptor superfamily, member |
| | | | | 12 (translocating chain-association membrane protein) |
| CTGCCAACTT | 90 | CTGCCAACTTCTAACCG | 345 | cofillin 1 (non-muscle) |
| AAGTGGATAG | 91 | AAGTGGATAGATACTTC | 346 | splicing factor proline/glutamine rich (poly- |
| | | | | pyrimidine tract-binding protein-associated) |
| CGTACTGAGC | 92 | CGTACTGAGCGCTTTGG | 347 | splicing factor proline/glutamine rich (poly- |
| | | | | pyrimidine tract-binding protein-associated) |
| CCGCTTACTC | 93 | CCGCTTACTCTGTTGGG | 348 | v-ets avian erythroblastosis virus E26 oncogene |
| | | | | binding 1 |
| GGGGCTTCTG | 94 | GGGGCTTCTGTAGCCCC | 349 | protease, cysteine, 1 (legumain) |
| CCCGTCCGGA | 95 | CCCGTCCGGAACGTCTA | 350 | ribosomal protein L13 |
| AGTTCCACCA | 96 | AGTTCCACCAGAAAGCC | 351 | chromosome 22 open reading frame 5 |
| GGCCTCCAGC | 97 | GGCCTCCAGCCACCCAC | 352 | zinc finger protein 144 (Mel-18) |
| GGAGGCTGAG | 98 | GGAGGCTGAGGTGGGAG | 353 | degenerative spermatocyte (homolog Drosophilia; |
| | | | | lipid desaturase) |
| CAGAGGCGTC | 99 | CAGAGGCGTCCGCAGGT | 354 | eukaryotic translation initiation factor 2C, 2 |
| GACCAGCCTT | 100 | GACCAGCCTTCAGATGG | 355 | mitochondrial ribosomal protein L45 |
| GAGGATGGTG | 101 | GAGGATGGTGTCCTGAG | 356 | prostate tumor over expressed gene 1 |
| TCGTCGCAGA | 102 | TCGTCGCAGAAGGCGCT | 357 | NADH dehydrogenase (ubiquinone) 1 alpha subcomplex, |
| | | | | 7 (14.5 kD, B14.5a) |
| GGGGCTGCCC | 103 | GGGGCTGCCCAGCTGGA | 358 | tumor endothelial marker 1 precursor |
| CTGTACATAC | 104 | CTGTACATACTTTTTGG | 359 | NS1-binding protein |
| GCGACGAGGC | 105 | GCGACGAGGCGCGCTGG | 360 | ribosomal protein L38 |
| GCCAAGTGAA | 106 | GCCAAGTGAACTGTGGC | 361 | tuftelin-interacting protein |
| AAGATAAACT | 107 | AAGATAAACTCTGGGCC | 362 | HLA class II region expressed gene KE2 |
| GAGAGTGTAC | 108 | GAGAGTGTACTGGCACT | 363 | translocase of inner mitochondrial membrane 17 |
| | | | | homolog A (yeast) |
| CCACTGCACT | 109 | CCACTGCACTCCGGCCT | 364 | sudD (suppressor of bimD6, Aspergillus nidulans) |
| | | | | homolog |
| CCACCCTCAC | 110 | CCACCCTCACACACACA | 365 | heparan sulfate proteoglycan 2 (perlecan) |
| CAGACCATTG | 111 | CAGACCATTGTTTGATC | 366 | SEC24 (S. cerevisiae) related gene family, member A |
| GGGAGCTGCG | 112 | GGGAGCTGCGCCAACGG | 367 | NADH dehydrogenase (ubiquinone) Fe—S protein 7 |
| | | | | (20 kD) (NADH-coenzyme Q reductase) |
| GGGATTTCTG | 113 | GGGATTTCTGTGTCTGC | 368 | DNA segment on chromosome X and Y (unique) 155 |
| | | | | expressed sequence |
| CTTCCAGCTA | 114 | CTTCCAGCTAACAGGTC | 369 | annexin A2 |
| CAGAAACAGA | 115 | CAGAAACAGACTGGGGG | 370 | Homo sapiens clone 24670 mRNA sequence |
| TCTGTGCTCA | 116 | TCTGTGCTCAGGAAGAG | 371 | hypothetical protein |
| TGCAATAGGT | 117 | TGCAATAGGTGAGAGAA | 372 | matrix metalloproteinase 10 (stromelysin 2) |
| ATGGCCAACT | 118 | ATGGCCAACTTCCACCT | 373 | KIAA1049 protein |
| TCACACAGTG | 119 | TCACACAGTGCCTGTCG | 374 | G protein-coupled receptor |
| GGCTTAGGAT | 120 | GGCTTAGGATGTGAATG | 375 | hypothetical protein FLJ20401 |
| GGGAGGGGTG | 121 | GGGAGGGGTGGGGGGTG | 376 | matrix metalloproteinase 14 (membrane-inserted) |
| GAAGTAGAAG | 122 | GAAGTAGAAGGTAAGGA | 377 | KIAA0470 gene product |
| CACCCTGTAC | 123 | CACCCTGTACAGTTGCC | 378 | solute carrier family 29 (nucleoside transporters), |
| | | | | member 1 |
| ATGTTTACAA | 124 | ATGTTTACAAGATGGCG | 379 | stanniocalcin 1 |
| CAAACTGGTC | 125 | CAAACTGGTCTAGGTCA | 380 | stanniocalcin 1 |
| GTAATGACAG | 126 | GTAATGACAGATGCAAG | 381 | stanniocalcin 1 |
| ACCTGCCGAC | 127 | ACCTGCCGACAGTGTTG | 382 | tumor suppressor deleted in oral cancer-related 1 |
| TGATGCGCGC | 128 | TGATGCGCGCTTTGTTG | 383 | tumor suppressor deleted in oral cancer-related 1 |
| TGGCCCCAGG | 129 | TGGCCCCAGGTGCCACC | 384 | apolipoprotein C-1 |
| GCCTGCTGGG | 130 | GCCTGCTGGGCTTGGCT | 385 | glutathione peroxidase 4 (phospholipid |
| | | | | hydroperoxidase) |
| TGCCTGTGGT | 131 | TGCCTGTGGTCCCAGCT | 386 | ETSs |
| GAGGGTATAC | 132 | GAGGGTATACTGAGGGG | 387 | transcription factor binding to IGHM enhancer 3 |
| GGAGCCAGCT | 133 | GGAGCCAGCTGACCTGC | 388 | hypothetical protein DKFZp762A227 |
| GAGCCTCAGG | 134 | GAGCCTCAGGTGCTCCC | 389 | hypothetical protein FLJ22362 |
| TACTTCACAT | 135 | TACTTCACATACAGTGC | 390 | CD59 antigen p18-20 (antigen identified by monoclonal |
| | | | | antibodies 16.3A5, EJ16, EJ30, EL32 and G344) |
| TAATCCCAGC | 136 | TAATCCCAGCACTTTGG | 391 | PRO0628 protein |
| CACCTTCCAG | 137 | CACCTTCCAGCCCGGGG | 392 | melanoma-associated antigen recognised by cytotoxic |
| | | | | T lymphocytes |
| GAGTCTGTTC | 138 | GAGTCTGTTCGTGACTC | 393 | LOC88745 |
| GGATTTTGGT | 139 | GGATTTTGGTCTCTGTC | 394 | Homo sapiens beta-1,3-galactosyltransferase-6 |
| | | | | (B3GALT6) mRNA |
| TGCCTGTAGT | 140 | TGCCTGTAGTCCTAGTT | 395 | sprouty (Drosophila) homolog 4 |
| TTACAAACAG | 141 | TTACAAACAGAAAAGCT | 396 | sprouty (Drosophila) homolog 4 |
| TCTTCTTTCA | 142 | TCTTCTTTCAGAATGGG | 397 | Homo sapiens mRNA; cDNA DKFZp434E1515 (from clone |
| AGCACATTTG | 143 | AGCACATTTGATATAGC | 398 | coactosin-like protein |
| CAGGGCTCGC | 144 | CAGGGCTCGCGTGCGGG | 399 | hypothetical protein FLJ21865 |
| GCTGGTCCCA | 145 | GCTGGTCCCAGGGCCAG | 400 | ESTs, Weakly similar to T31613 hypothetical protein |
| | | | | Y50E8A.i-Caenorhabditis elegans [C. elegans] |
| TCCACGCCCT | 146 | TCCACGCCCTTCCTGGC | 401 | KIAA0685 gene product |
| TTGCAATAGC | 147 | TTGCAATAGCAAAACCC | 402 | hypothetical protein FLJ10980 |
| AGGGCTTCCA | 148 | AGGGCTTCCAATGTGCT | 403 | ribosomal protein L10 |
| CTGGGTTAAT | 149 | CTGGGTTAATAAATTGC | 404 | ribosomal protein S19 |
| AACCTGGGAG | 150 | AACCTGGGAGGTGGAGG | 405 | ESTs |
| GGCAACGTGG | 151 | GGCAACGTGGTAGAGGC | 406 | Huntingtin interacting protein K |
| GGATGCGCAG | 152 | GGATGCGCAGGGGAGGC | 407 | Homo sapiens mRNA full length insert cDNA clone |
| | | | | EUROIMAGE 50374 |
| CACCTGTAGT | 153 | CACCTGTAGTCCTAGCT | 408 | EST |
| GTGGTGGGCG | 154 | GTGGTGGGCGCCTGTAG | 409 | EST |
| GCAGGGTGGG | 155 | GCAGGGTGGGGAGGGG | 410 | v-akt murine thymoma viral oncogene homolog 2 |
| CAAGCATCCC | 156 | CAAGCATCCCCGTTCCA | 411 | EST |
| TGGGGGCCGA | 157 | TGGGGGCCGATGGGCAG | 412 | transducin-like enhancer of split 2, homolog of |
| | | | | Drosophila E(sp1) |
| TCAGTGTATT | 158 | TCAGTGTATTAAAACCC | 413 | KIAA1870 protein |
| GGCAAGCCCC | 159 | GGCAAGCCCCAGCGCCT | 414 | ribosomal protein L10a |
| CCTAGCTGGA | 160 | CCTAGCTGGATTGCAGA | 415 | peptidylprolyl isomerase A (cyclophilin A) |
| GCAAAACCCT | 161 | GCAAAACCCTGCTCTCC | 416 | ESTs, Weakly similar to ubiquitous TPR motif, Y |
| | | | | isoform [H. sapiens] |
| GCTGGTTCCT | 162 | GCTGGTTCCTGAGTGGC | 417 | hypothetical protein FLJ23239 |
| GCACCTCAGC | 163 | GCACCTCAGCCAGGGGT | 418 | hypothetical protein DKFZp761H221 |
| ACCAGCTGTC | 164 | ACCAGCTGTCCAGGGGC | 419 | KIAA1887 protein |
| TTTGAATCAG | 165 | TTTGAATCAGTGCTAGA | 420 | Homo sapiens mRNA full length insert cDNA clone |
| | | | | EUROIMAGE 701679 |
| AGACTAGGGG | 166 | AGACTAGGGGCCGGAGC | 421 | Homo sapiens cDNA FLJ30634 fis, clone CTONG2002453 |
| AGCTCAGTGA | 167 | AGCTCAGTGAGAAGGGC | 422 | Homo sapiens cDNA FLJ32203 fis, clone PLACE6003038, |
| | | | | weakly similar to ZINC FINGER PROTEIN 84 |
| GGCCAACATT | 168 | GGCCAACATTTGGTCCA | 423 | Homo sapiens mRNA full length insert cDNA clone |
| | | | | EUROIMAGE 1035904 |
| TTTGTGGGCA | 169 | TTTGTGGGCAGTCAGGC | 424 | hypothetical protein LOC57333 |
| ATTGTAGACA | 170 | ATTGTAGACAATGAGGG | 425 | myosin ID |
| CCCTAGGTTG | 171 | CCCTAGGTTGGGCCCCT | 426 | plexin B2 |
| AAATCACCAA | 172 | AAATCACCAATCAAGGC | 427 | lectin, galactoside-binding, soluble, 8 (galectin 8) |
| GGCTGCAGTC | 173 | GGCTGCAGTCTTCTTCC | 428 | double ring-finger protein, Dorfin |
| GTGGCAGGCG | 174 | GTGGCAGGCGCCTGTAG | 429 | DKFZP434B168 protein |
| TAAAGGCACA | 175 | TAAAGGCACAGTGGCTC | 430 | LIM domain binding 2 |
| GGCTCCTGGC | 176 | GGCTCCTGGCTCTGGAC | 431 | integrin beta 4 binding protein |
| ATATTAGGAA | 177 | ATATTAGGAAGTCGGGG | 432 | Synaptopodin |
| GCTTCAGTGG | 178 | GCTTCAGTGGGGGAGAG | 433 | ESTs |
| TGATTAAAAC | 179 | TGATTAAAACAAGTTGC | 434 | insulin induced gene 1 |
| AGCCACCACG | 180 | AGCCACCACGCCTGGTC | 435 | acetyl LDL receptor; SREC |
| GGCGGCTGCA | 181 | GGCGGCTGCAGAGCCTG | 436 | excision repair cross-complementing rodent repair |
| | | | | deficiency, complementation group 1 |
| | | | | (induces overlapping antisense sequence) |
| TGTTTGGGGG | 182 | TGTTTGGGGGCTTTTAG | 437 | hypothetical protein FLJ22329 |
| CCTGCCTCGT | 183 | CCTGCCTCGTAGTGAAG | 438 | schwannomin-interacting protein 1 |
| AGGCCTGGGC | 184 | AGGCCTGGGCCTCTGCG | 439 | PTEN induced putative kinase 1 |
| CAAAACTGTT | 185 | CAAAACTGTTTGTTGGC | 440 | myosin X |
| GAGAGGACAT | 186 | GAGAGGACATTGGAGGG | 441 | Homo sapiens cDNA FLJ32424 fis, clone SKMUS2000954, |
| | | | | moderately similar to Homo sapiens F-box protein |
| | | | | Fbx25 (FBX25) 97 |
| GAGTTAGGCA | 187 | GAGTTAGGCACTTCCTG | 442 | golgi phosphoprotein 1 |
| CCGTAGTGCC | 188 | CCGTAGTGCCTTTATGG | 443 | splicing factor, arginine/serine-rich 6 |
| CATAAACGGG | 189 | CATAAACGGGCACACCC | 444 | laminin, gamma 3 |
| TCCCTGGCAG | 190 | TCCCTGGCAGAGGGCTT | 445 | cysteine-rich protein 2 |
| GAGGCCATCC | 191 | GAGGCCATCCCCAACCC | 446 | U6 snRNA-associated Sm-like protein LSm7 |
| TTGCCTGGGA | 192 | TTGCCTGGGATGCTGGT | 447 | hypothetical protein FLJ10707 |
| CTGTCAGCGG | 193 | CTGTCAGCGGCTGCCCC | 448 | Homo sapiens, Similar to RIKEN cDNA 2310012N15 gene, |
| | | | | clone IMAGE: 3342825, mRNA, partial cds |
| AACGCGGCCA | 194 | AACGCGGCCAATGTGGG | 449 | macrophage migration inhibitory factor |
| | | | | (glycosylation-inhibiting factor) |
| GGTTTGGCTT | 195 | GGTTTGGCTTAGGCTGG | 450 | ubiquinol-cytochrome c reductase hinge protein |
| GATTTTTGTG | 196 | GATTTTTGTGGTGTGGG | 451 | gap junction protein, alpha 1, 43 kD (connexin 43) |
| GGCTGCCCTG | 197 | GGCTGCCCTGGGCAGCC | 452 | dihydropyrimidinase-like 3 |
| ATGGCAACAG | 198 | ATGGCAACAGAAACCAA | 453 | aquaporin 1 (channel-forming integral protein, 28 kD) |
| CGCTGTGGGG | 199 | CGCTGTGGGGTGCAGAC | 454 | protein expressed in thyroid |
| GGCAGCCAGA | 200 | GGCAGCCAGAGCTCCAA | 455 | macrophage myristoylated alanine-rich C kinase |
| | | | | substrate |
| AGAGCAAACC | 201 | AGAGCAAACCGTAGTCC | 456 | procollagen-lysine, 2-oxoglutarate 5-dioxygenase |
| | | | | (lysine hydroxylase, Ehlers-Danlos syndrome type VI) |
| TTTCCCTCAA | 202 | TTTCCCTCAAAGACTCT | 457 | protease, serine, 11 (IGF binding) |
| TCCCCGTGGC | 203 | TCCCCGTGGCTGTGGGG | 458 | 24-dehydrocholesterol reductase |
| TTCTCCCAAA | 204 | TTCTCCCAAATACCGTT | 459 | collagen, type IV, alpha 2 |
| GGCTGGGGGC | 205 | GGCTGGGGGCCAGGGCT | 460 | profilin 1 |
| CCCTACCCTG | 206 | CCCTACCCTGTTACCTT | 461 | apolipoprotein D |
| TAGGACCCTG | 207 | TAGGACCCTGCAGGGGG | 462 | hyaluronoglucosaminidase 2 |
| GTTTTTGCTT | 208 | GTTTTTGCTTCAGCGGC | 463 | hypothetical protein FLJ22678 |
| CTTGATTCCC | 209 | CTTGATTCCCACGCTAC | 464 | quiescin Q6 |
| GCTTGGCTCC | 210 | GCTTGGCTCCCAAAGGG | 465 | ras homolog gene family, member A |
| GGTGGCACTC | 211 | GGTGGCACTCAGTCTCT | 466 | ras homolog gene family, member A |
| ACCTGTGACC | 212 | ACCTGTGACCAGCACTG | 467 | plasminogen activator, urokinase |
| ACTGAGGAAA | 213 | ACTGAGGAAAGGAGCTC | 468 | insulin-like growth factor binding protein 3 |
| TGCAGCGCCT | 214 | TGCAGCGCCTGCGGCCT | 469 | uridine phosphorylase |
| CTGGGGGGAA | 215 | CTGGGGGGAAGGGACTG | 470 | KIAA0638 protein |
| GTGCTATTCT | 216 | GTGCTATTCTGGGGCTG | 471 | B7 homolog 3 |
| GGAGGGGGCT | 217 | GGAGGGGGCTTGAAGCC | 472 | lamin A/C |
| GTGCCTGAGA | 218 | GTGCCTGAGAGGCAGGC | 473 | lamin A/C |
| TCACAGGGTC | 219 | TCACAGGGTCCCCGGGG | 474 | lamin A/C |
| GGGCTCCCTG | 220 | GGGCTCCCTGGCCCTGG | 475 | regulator of G-protein signalling 12 |
| GCCCCAGGTA | 221 | GCCCCAGGTAGGGGGAC | 476 | proteasome (prosome, macropain) 26S subunit, |
| | | | | non-ATPase, 8 |
| GAAAGTGGCT | 222 | GAAAGTGGCTGTCCTGG | 477 | Homo sapiens, Similar to RIKEN cDNA 5730528L 13 gene, |
| | | | | clone MGC: 17337 IMAGE: 4213591, mRNA, complete cds |
| TCCCTGGCTG | 223 | TCCCTGGCTGTTGAGGC | 478 | prosaposin (variant Gaucher disease and variant |
| | | | | metachromatic |
| ACAGAGCACA | 224 | ACAGAGCACAGCTGCCC | 479 | laminin, alpha 4 |
| CTTTGCACTC | 225 | CTTTGCACTCTCCTTTG | 480 | transcription elongation factor A (SII), 1 |
| ATGCTCCCTG | 226 | ATGCTCCCTGAGGAGCT | 481 | lectin, galactoside-binding, soluble, 3 binding |
| | | | | protein |
| CCGTCCAAGG | 227 | CCGTCCAAGGGTCCGCT | 482 | ribosomal protein S16 |
| GGGCCCCCTG | 228 | GGGCCCCCTGGGCAGTG | 483 | glycophorin C (Gerbich blood group) |
| CTTATGCTGC | 229 | CTTATGCTGCTGGTGCC | 484 | endothelin receptor type B |
| GGTTATTTTG | 230 | GGTTATTTTGGAGTGTA | 485 | serine (or cysteine) proteinase inhibitor, clade E |
| | | | | (nexin, plasminogen activator inhibitor type 1), |
| | | | | member 1 |
| GCCTGTCCCT | 231 | GCCTGTCCCTCCAAGAC | 486 | Biglycan |
| AAGATGAGGG | 232 | AAGATGAGGGGGCAGGC | 487 | small nuclear ribonucleoprotein polypeptide B″ |
| CCAACAAGAA | 233 | CCAACAAGAATGCATTG | 488 | transmembrane 4 superfamily member 2 |
| AAGGATGCGG | 234 | AAGGATGCGGTGATGGC | 489 | TAF11 RNA polymerase II, TATA box binding protein |
| | | | | (TBP)-associated factor, 28 kD |
| TGTCATCACA | 235 | TGTCATCACAGACACTT | 490 | lysyl oxidase-like 2 |
| CAGGCTTTTT | 236 | CAGGCTTTTTGGCTTCC | 491 | SRY (sex determining region Y)-box 4 |
| TCAAGTTCAC | 237 | TCAAGTTCACTGCCTGT | 492 | SOX4 SRY (sex determining region Y)-box 4 |
| TCCCTGGGCA | 238 | TCCCTGGGCAGCTTCAG | 493 | SRY (sex determining region Y)-box 4 |
| CAGGAGTTCA | 239 | CAGGAGTTCAAAGAAGG | 494 | actin related protein 2/3 complex, subunit 2 (34 kD) |
| CAGGTGGTTC | 240 | CAGGTGGTTCTGCCATC | 495 | Homo sapiens cDNA: FLJ23507 fis, clone LNG03128 |
| GCCCACATCC | 241 | GCCCACATCCGCTGAGG | 496 | hypothetical protein FLJ12442 |
| GCTGGGGTGG | 242 | GCTGGGGTGGGGGTGG | 497 | Fas (TNFRSF6)-associated via death domain |
| GACCTCCTGC | 243 | GACCTCCTGCCCTGGGG | 498 | mitogen-activated protein kinase kinase kinase 11 |
| AGTGAATAAA | 244 | AGTGAATAAATGTCTTG | 499 | TEK tyrosine kinase, endothelial (venous |
| | | | | malformations, multiple cutaneous and mucosal) |
| AAGGTTCTTC | 245 | AAGGTTCTTCTCAAGGG | 500 | insulin receptor |
| AGCCTGGACT | 246 | AGCCTGGACTGAGCCAC | 501 | cell membrane glycoprotein, 110000M(r) |
| | | | | (surface antigen) |
| CAACCCAGAT | 247 | CAACCCAGATTGGGGTG | 502 | Homo sapiens cDNA FLJ11863 fis, clone HEMBA1006926 |
| TGCTTCTGCC | 248 | TGCTTCTGCCACCCTGC | 503 | jagged 1 (Alagille syndrome) |
| CAGGTGACAA | 249 | CAGGTGACAAGGGCCCT | 504 | KIAA0304 gene product |
| GGCCGGGGGC | 250 | GGCCGGGGGCAGTTCTC | 505 | pre-B-cell leukemia transcription factor 2 |
| GTGCGCTAGG | 251 | GTGCGCTAGGGCCCCGG | 506 | Homo sapiens cDNA FLJ31238 fis, clone KIDNE2004864 |
| AGGCTGTCCA | 252 | AGGCTGTCCAGGCTCTG | 507 | p53-induced protein |
| TGTTATGTCC | 253 | TGTTATGTCCATTTTGC | 508 | complement component 1, q subcomponent, receptor 1 |
| TTTCCCAAAC | 254 | TTTCCCAAACTGTGAGG | 509 | complement component 1, q subcomponent, receptor 1 |
| GGGGATGGGG | 255 | GGGGATGGGGTACTGCC | 510 | Homo sapiens, clone IMAGE: 3908182, mRNA, partial cds |

TABLE 2

| SEQ ID NO: | Unigene ID | OMIMID | gene symbol | locuslink id | Cellular Component |
|---|---|---|---|---|---|
| 33 | Hs.102135 | 300090 | SS | 6748 | endoplasmic reticulum, membrane |
| 34 | Hs.103180 | | | | |
| 35 | Hs.105850 | | KIAA0404 | 23130 | |
| 36 | Hs.107019 | 602388 | SPK | 8189 | cytoplasm, nucleoplasm |
| 37 | Hs.107125 | | | | membrane |
| 38 | Hs.107809 | | KIAA0726 | 9746 | membrane |
| 39 | Hs.109276 | | | | |
| 40 | Hs.1103 | 190180 | TGFB1 | 7040 | |
| 41 | Hs.110443 | | | | |
| 42 | Hs.110950 | | | | |
| 43 | Hs.110964 | | | | |
| 44 | Hs.111039 | 160993 | NMT1 | 4836 | |
| 45 | Hs.11114 | | DJ1181N3 | 58476 | |
| 46 | Hs.111611 | | RPL27 | 6155 | intracellular, ribosome |
| 47 | Hs.111779 | 182120 | SPARC | 6678 | basement membrane |
| 48 | Hs.111779 | 182120 | SPARC | 6678 | basement membrane |
| 49 | Hs.111779 | 182120 | SPARC | 6678 | basement membrane |
| 50 | Hs.111779 | 182120 | SPARC | 6678 | basement membrane |
| 51 | Hs.111988 | | | | |
| 52 | Hs.112238 | | | | |
| 53 | Hs.112844 | | | | |
| 54 | Hs.11669 | 601033 | LAMA5 | 3911 | basement lamina |
| 55 | Hs.118126 | 256540 | PPGB | 5476 | endoplasmic reticulum, lysosome |
| 56 | Hs.118893 | 600134 | D2S448 | 7837 | cellular_component unknown |
| 57 | Hs.118893 | 600134 | D2S448 | 7837 | cellular_component unknown |
| 58 | Hs.119120 | 605568 | SMURF1 | 57154 | intracellular |
| 59 | Hs.119129 | 120130 | COL4A1 | 1282 | collagen |
| 60 | Hs.119129 | 120130 | COL4A1 | 1282 | collagen |
| 61 | Hs.119129 | 120130 | COL4A1 | 1282 | collagen |
| 62 | Hs.119206 | 602867 | IGFBP7 | 3490 | extracellular |
| 63 | Hs.124 | | | | |
| 64 | Hs.125359 | 188230 | THY1 | 7070 | integral plasma membrane protein |
| 65 | Hs.127824 | | | | |
| 66 | Hs.13011 | | GTPBP2 | 54676 | |
| 67 | Hs.13350 | | | | |
| 68 | Hs.136164 | | | | |
| 69 | Hs.143897 | 603009 | DYSF | 8291 | plasma membrane |
| 70 | Hs.149098 | 602127 | SMTN | 6525 | actin cytoskeleton |
| 71 | Hs.149666 | 135620 | ITGA5 | 3678 | cytoskeleton, extracellular matrix, |
| 72 | Hs.150580 | | SUI1 | 10209 | cellular_component unknown |
| 73 | Hs.15165 | | | | |
| 74 | Hs.151738 | 120361 | MMP9 | 4318 | extracellular matrix, extracellular space |
| 75 | Hs.155048 | 111200 | LU | 4059 | integral plasma membrane protein, |
| 76 | Hs.155223 | 603665 | STC2 | 8614 | |
| 77 | Hs.155396 | 600492 | NFE2L2 | 4780 | nucleus |
| 78 | Hs.155894 | 176885 | PTPN1 | 5770 | cytoplasm, soluble fraction |
| 79 | Hs.158237 | 604042 | ITGA10 | 8515 | cytoskeleton, extracellular matrix, |
| 80 | Hs.159263 | 120240 | COL6A2 | 1292 | extracellular matrix |
| 81 | Hs.16007 | | | | |
| 82 | Hs.160958 | 605065 | CDC37 | 11140 | |
| 83 | Hs.16450 | | | | |
| 84 | Hs.172813 | 605477 | P85SPR | 8874 | |
| 85 | Hs.173724 | 123280 | CKB | 1152 | cytoplasm |
| 86 | Hs.173739 | | | | |
| 87 | Hs.177596 | | | | |
| 88 | Hs.17839 | | GG2 | 25816 | |
| 89 | Hs.180338 | 603366 | TNFRSF12 | 8718 | integral plasma membrane protein, |
| 90 | Hs.180370 | 601442 | CFL1 | 1072 | cytoskeleton, nucleus |
| 91 | Hs.180610 | 605199 | SFPQ | 6421 | nucleus |
| 92 | Hs.180610 | 605199 | SFPQ | 6421 | nucleus |
| 93 | Hs.18063 | 164720 | ETS1 | 2113 | nucleus |
| 94 | Hs.18069 | 602620 | PRSC1 | 5641 | |
| 95 | Hs.180842 | 113703 | RPL13 | 6137 | cytosolic ribosome, intracellular |
| 96 | Hs.182626 | | | | |
| 97 | Hs.184669 | 600346 | ZNF144 | 7703 | nucleus |
| 98 | Hs.185973 | | DEGS | 8560 | endoplasmic reticulum, integral plasma |
| 99 | Hs.193053 | 606229 | EIF2C2 | 27161 | cellular_component unknown |
| 100 | Hs.19347 | | MRPL45 | 84311 | mitochondrion |
| 101 | Hs.19555 | | | | |
| 102 | Hs.19561 | 602139 | NDUFA7 | 4701 | membrane fraction, mitochondrion, |
| 103 | Hs.195727 | 606064 | TEM1 | 57124 | extracellular matrix |
| 104 | Hs.197298 | | NS1 | 10625 | spliceosome, transcription factor |
| 105 | Hs.2017 | 604182 | RPL38 | 6169 | 60S ribosomal subunit, intracellular, |
| 106 | Hs.20225 | | | | |
| 107 | Hs.205736 | 605660 | HKE2 | 10471 | prefoldin |
| 108 | Hs.20716 | 605057 | TIM17 | 10440 | integral plasma membrane protein, |
| 109 | Hs.209061 | 603579 | SUDD | 8780 | |
| 110 | Hs.211573 | 142461 | HSPG2 | 3339 | basement membrane, extracellular |
| 111 | Hs.211612 | | SEC24A | 10802 | COPII vesicle coat, endoplasmic |
| 112 | Hs.211914 | 601825 | NDUFS7 | 4727 | mitochondrion, NADH dehydrogenase |
| 113 | Hs.21595 | 312095 | DXYS155E | 8227 | cellular_component unknown |
| 114 | Hs.217493 | 151740 | ANXA2 | 302 | plasma membrane, soluble fraction |
| 115 | Hs.21906 | | | | |
| 116 | Hs.22129 | | | | |
| 117 | Hs.2258 | 185260 | MMP10 | 4319 | extracellular matrix, extracellular space |
| 118 | Hs.227835 | | | | |
| 119 | Hs.23016 | | RDC1 | 57007 | integral membrane protein, membrane |
| 120 | Hs.233955 | | | | |
| 121 | Hs.2399 | 600754 | MMP14 | 4323 | extracellular matrix, integral plasma |
| 122 | Hs.25132 | | | | |
| 123 | Hs.25450 | 602193 | SLC29A1 | 2030 | integral plasma membrane protein, |
| 124 | Hs.25590 | 601185 | STC1 | 6781 | |
| 125 | Hs.25590 | 601185 | STC1 | 6781 | |
| 126 | Hs.25590 | 601185 | STC1 | 6781 | |
| 127 | Hs.25664 | | DOC | 10263 | |
| 128 | Hs.25664 | | DOC | 10263 | |
| 129 | Hs.268571 | 107710 | APOC1 | 341 | |
| 130 | Hs.2706 | 138322 | GPX4 | 2879 | mitochondrion |
| 131 | Hs.272106 | | | | |
| 132 | Hs.274184 | 314310 | TFE3 | 7030 | nucleus |
| 133 | Hs.274453 | | | | |
| 134 | Hs.27836 | | | | |
| 135 | Hs.278573 | 107271 | CD59 | 966 | membrane fraction, plasma membrane |
| 136 | Hs.278941 | | | | |
| 137 | Hs.279869 | 604853 | MAAT1 | 10573 | |
| 138 | Hs.283636 | | | | |
| 139 | Hs.284284 | | | | |
| 140 | Hs.285814 | | | | |
| 141 | Hs.285814 | | | | |
| 142 | Hs.287830 | | | | |
| 143 | Hs.289092 | | CLP | 23406 | intracellular |
| 144 | Hs.29288 | | | | |
| 145 | Hs.296234 | | | | |
| 146 | Hs.296406 | | | | |
| 147 | Hs.29716 | | | | |
| 148 | Hs.29797 | 312173 | RPL10 | 6134 | 60S ribosomal subunit, intracellular, |
| 149 | Hs.298262 | 603474 | RPS19 | 6223 | 40S ribosomal subunit, intracellular, |
| | | | | | ribosome |
| 150 | Hs.299257 | | | | |
| 151 | Hs.300954 | | | | |
| 152 | Hs.302741 | | | | |
| 153 | Hs.311780 | | | | |
| 154 | Hs.312191 | | | | |
| 155 | Hs.326445 | 164731 | AKT2 | 208 | |
| 156 | Hs.327884 | | | | |
| 157 | Hs.332173 | 601041 | TLE2 | 7089 | nucleus |
| 158 | Hs.334604 | | KIAA1870 | 85301 | collagen |
| 159 | Hs.334895 | | RPL10A | 4736 | 60S ribosomal subunit, intracellular, |
| 160 | Hs.342389 | 123840 | PPIA | 5478 | cytoplasm |
| 161 | Hs.344224 | | | | |
| 162 | Hs.34516 | | | | |
| 163 | Hs.347297 | | | | |
| 164 | Hs.348428 | | | | |
| 165 | Hs.348967 | | | | |
| 166 | Hs.350065 | | | | |
| 167 | Hs.351706 | | | | |
| 168 | Hs.36353 | | | | |
| 169 | Hs.39619 | | LOC57333 | 57333 | |
| 170 | Hs.39871 | 606539 | MYO1D | 4642 | myosin |
| 171 | Hs.3989 | 604293 | PLXNB2 | 23654 | membrane |
| 172 | Hs.4082 | 606099 | LGALS8 | 3964 | extracellular space |
| 173 | Hs.48320 | | DORFIN | 25897 | centrosome |
| 174 | Hs.48604 | | | | |
| 175 | Hs.4980 | 603450 | LDB2 | 9079 | nucleus |
| 176 | Hs.5215 | 602912 | ITGB4BP | 3692 | extrinsic plasma membrane protein, |
| 177 | Hs.5307 | | | | |
| 178 | Hs.54828 | | | | |
| 179 | Hs.56205 | 602055 | INSIG1 | 3638 | |
| 180 | Hs.57735 | | SREC | 8578 | membrane |
| 181 | Hs.59544 | 126380 | ERCC1 | 2067 | nucleus |
| 182 | Hs.61478 | | | | |
| 183 | Hs.61490 | | | | |
| 184 | Hs.6163 | | PINK1 | 65018 | |
| 185 | Hs.61638 | | | | |
| 186 | Hs.61661 | | | | |
| 187 | Hs.6831 | | | | |
| 188 | Hs.6891 | 601944 | SFRS6 | 6431 | nucleus |
| 189 | Hs.69954 | 604349 | LAMC3 | 10319 | extracellular matrix, membrane |
| 190 | Hs.70327 | 601183 | CRIP2 | 1397 | |
| 191 | Hs.70830 | | LOC51690 | 51690 | nucleus, small nucleolar |
| 192 | Hs.7187 | | | | |
| 193 | Hs.7247 | | | | |
| 194 | Hs.73798 | 153620 | MIF | 4282 | extracellular space |
| 195 | Hs.73818 | | UQCRH | 7388 | mitochondrial electron transport chain |
| 196 | Hs.74471 | 121014 | GJA1 | 2697 | connexon, integral plasma membrane |
| 197 | Hs.74566 | 601168 | DPYSL3 | 1809 | |
| 198 | Hs.74602 | 107776 | AQP1 | 358 | integral plasma membrane protein, |
| 199 | Hs.7486 | | | | |
| 200 | Hs.75061 | | MLP | 65108 | |
| 201 | Hs.75093 | 153454 | PLOD | 5351 | endoplasmic reticulum |
| 202 | Hs.75111 | 602194 | PRSS11 | 5654 | extracellular space |
| 203 | Hs.75616 | | | | |
| 204 | Hs.75617 | 120090 | COL4A2 | 1284 | collagen, collagen type IV |
| 205 | Hs.75721 | 176610 | PFN1 | 5216 | actin cytoskeleton |
| 206 | Hs.75736 | 107740 | APOD | 347 | extracellular space |
| 207 | Hs.76873 | 603551 | HYAL2 | 8692 | lysosome |
| 208 | Hs.7718 | | | | |
| 209 | Hs.77266 | 603120 | QSCN6 | 5768 | |
| 210 | Hs.77273 | 165390 | ARHA | 387 | cytoskeleton |
| 211 | Hs.77273 | 165390 | ARHA | 387 | cytoskeleton |
| 212 | Hs.77274 | 191840 | PLAU | 5328 | extracellular space |
| 213 | Hs.77326 | 146732 | IGFBP3 | 3486 | extracellular space |
| 214 | Hs.77573 | 191730 | UP | 7378 | |
| 215 | Hs.77864 | | | | |
| 216 | Hs.77873 | 605715 | B7 | 80381 | cellular_component unknown |
| 217 | Hs.77886 | 150330 | LMNA | 4000 | lamin, nuclear lamina, nucleus |
| 218 | Hs.77886 | 150330 | LMNA | 4000 | lamin, nuclear lamina, nucleus |
| 219 | Hs.77886 | 150330 | LMNA | 4000 | lamin, nuclear lamina, nucleus |
| 220 | Hs.78281 | 602512 | RGS12 | 6002 | extrinsic plasma membrane protein, |
| 221 | Hs.78466 | | PSMD8 | 5714 | 19S proteasome regulatory particle |
| 222 | Hs.78531 | | | | |
| 223 | Hs.78575 | 176801 | PSAP | 5660 | extracellular space, integral membrane |
| 224 | Hs.78672 | 600133 | LAMA4 | 3910 | basement lamina |
| 225 | Hs.78869 | 601425 | TCEA1 | 6917 | nucleus |
| 226 | Hs.79339 | 600626 | LGALS3BP | 3959 | extracellular space, membrane |
| 227 | Hs.80617 | 603675 | RPS16 | 6217 | 40S ribosomal subunit, intracellular, |
| 228 | Hs.81994 | 110750 | GYPC | 2995 | integral plasma membrane protein, |
| 229 | Hs.82002 | 131244 | EDNRB | 1910 | integral plasma membrane protein, |
| 230 | Hs.82085 | 173360 | SERPINE1 | 5054 | |
| 231 | Hs.821 | 301870 | BGN | 633 | extracellular matrix |
| 232 | Hs.82575 | 603520 | SNRPB2 | 6629 | nucleus, snRNP U2e |
| 233 | Hs.82749 | 300096 | TM4SF2 | 7102 | integral plasma membrane protein, |
| 234 | Hs.83126 | 600772 | TAF2I | 6882 | nucleus, TFIID complex |
| 235 | Hs.83354 | | LOXL2 | 4017 | extracellular space, membrane |
| 236 | Hs.83484 | 184430 | SOX4 | 6659 | nucleus |
| 237 | Hs.83484 | | | | |
| 238 | Hs.83484 | 184430 | SOX4 | 6659 | nucleus |
| 239 | Hs.83583 | 604224 | ARPC2 | 10109 | actin cytoskeleton, Arp2/3 protein |
| 240 | Hs.84063 | | | | |
| 241 | Hs.84753 | | | | |
| 242 | Hs.86131 | 602457 | FADD | 8772 | cytoplasm |
| 243 | Hs.89449 | 600050 | MAP3K11 | 4296 | |
| 244 | Hs.89640 | 600221 | TEK | 7010 | integral plasma membrane protein, |
| 245 | Hs.89695 | 147670 | INSR | 3643 | integral plasma membrane protein, |
| 246 | Hs.90107 | | GP110 | 11047 | integral plasma membrane protein, |
| 247 | Hs.9096 | | | | |
| 248 | Hs.91143 | 601920 | JAG1 | 182 | membrane |
| 249 | Hs.92236 | | KIAA0304 | 9757 | nucleus |
| 250 | Hs.93728 | 176311 | PBX2 | 5089 | nucleus |
| 251 | Hs.9408 | | | | |
| 252 | Hs.96908 | | PIG11 | 9537 | |
| 253 | Hs.97199 | 120577 | C1QR | 22918 | integral plasma membrane protein, |
| 254 | Hs.97199 | 120577 | C1QR | 22918 | integral plasma membrane protein, |
| 255 | Hs.99093 | | | | |

Isolated and purified nucleic acids, according to the present invention are those which are not linked to those genes to which they are linked in the human genome. Moreover, they are not present in a mixture such as a library containing a multitude of distinct sequences from distinct genes. They may be, however, linked to other genes such as vector sequences or sequences of other genes to which they are not naturally adjacent. Tags disclosed herein, because of the way that they were made, represent sequences which are 3′ of the 3′ most restriction enzyme recognition site for the tagging enzyme used to generate the SAGE tags. In this case, the tags are 3′ of the most 3′ most NlaIII site in the cDNA molecules corresponding to mRNA. Nucleic acids corresponding to tags may be RNA, cDNA, or genomic DNA, for example. Such corresponding nucleic acids can be determined by comparison to sequence databases to determine sequence identities. Sequence comparisons can be done using any available technique, such as BLAST, available from the National Library of Medicine, National Center for Biotechnology Information. Tags can also be used as hybridization probes to libraries of genomic or cDNA to identify the genes from which they derive. Thus, using sequence comparisons or cloning, or combinations of these methods, one skilled in the art can obtain full-length nucleic acid sequences. Genes corresponding to tags will contain the sequence of the tag at the 3′ end of the coding sequence or of the 3′ untranslated region (UTR), 3′ of the 3′ most recognition site in the cDNA for the restriction endonuclease which was used to make the tags. The nucleic acids may represent either the sense or the anti-sense strand. Nucleic acids and proteins although disclosed herein with sequence particularity, may be derived from a single individual. Allelic variants which occur in the population of humans are included within the scope of such nucleic acids and proteins. Those of skill in the art are well able to identify allelic variants as being the same gene or protein. Given a nucleic acid, one of ordinary skill in the art can readily determine an open reading frame present, and consequently the sequence of a polypeptide encoded by the open reading frame and, using techniques well known in the art, express such protein in a suitable host. Proteins comprising such polypeptides can be the naturally occurring proteins, fusion proteins comprising exogenous sequences from other genes from humans or other species, epitope tagged polypeptides, etc. Isolated and purified proteins are not in a cell, and are separated from the normal cellular constituents, such as nucleic acids, lipids, etc. Typically the protein is purified to such an extent that it comprises the predominant species of protein in the composition, such as greater than 50, 60 70, 80, 90, or even 95% of the proteins present.

Using the proteins according to the invention, one of ordinary skill in the art can readily generate antibodies which specifically bind to the proteins. Such antibodies can be monoclonal or polyclonal. They can be chimeric, humanized, or totally human. Any functional fragment or derivative of an antibody can be used including Fab, Fab′, Fab2, Fab′2, and single chain variable regions. So long as the fragment or derivative retains specificity of binding for the endothelial marker protein it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific.

Techniques for making such partially to fully human antibodies are known in the art and any such techniques can be used. According to one particularly preferred embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. See for example, Nina D. Russel, Jose R. F. Corvalan, Michael L. Gallo, C. Geoffrey Davis, Liise-Anne Pirofski. Production of Protective Human Antipneumococcal Antibodies by Transgenic Mice with Human Immunoglobulin Loci Infection and Immunity April 2000, p. 1820-1826; Michael L. Gallo, Vladimir E. Ivanov, Aya Jakobovits, and C. Geoffrey Davis. The human immunoglobulin loci introduced into mice: V (D) and J gene segment usage similar to that of adult humans European Journal of Immunology 30: 534-540, 2000; Larry L. Green. Antibody engineering via genetic engineering of the mouse: XenoMouse strains are a vehicle for the facile generation of therapeutic human monoclonal antibodies Journal of Immunological Methods 231 11-23, 1999; Yang X-D, Corvalan J R F, Wang P, Roy C M-N and Davis C G. Fully Human Anti-interleukin-8 Monoclonal Antibodies: Potential Therapeutics for the Treatment of Inflammatory Disease States. Journal of Leukocyte Biology Vol. 66, pp 401-410 (1999); Yang X-D, Jia X-C, Corvalan J R F, Wang P, C G Davis and Jakobovits A. Eradication of Established Tumors by a Fully Human Monoclonal Antibody to the Epidermal Growth Factor Receptor without Concomitant Chemotherapy. Cancer Research Vol. 59, Number 6, pp 1236-1243 (1999); Jakobovits A. Production and selection of antigen-specific fully human monoclonal antibodies from mice engineered with human Ig loci. Advanced Drug Delivery Reviews Vol. 31, pp: 33-42 (1998); Green L and Jakobovits A. Regulation of B cell development by variable gene complexity in mice reconstituted with human immunoglobulin yeast artificial chromosomes. J. Exp. Med. Vol. 188, Number 3, pp: 483-495 (1998); Jakobovits A. The long-awaited magic bullets: therapeutic human monoclonal antibodies from transgenic mice. Exp. Opin. Invest. Drugs Vol. 7 (4), pp: 607-614 (1998); Tsuda H, Maynard-Currie K, Reid L, Yoshida T, Edamura K, Maeda N, Smithies O, Jakobovits A. Inactivation of Mouse HPRT locus by a 203-bp retrotransposon insertion and a 55-kb gene-targeted deletion: establishment of new HPRT-Deficient mouse embryonic sGEM cell lines. Genomics Vol. 42, pp: 413-421 (1997); Sherman-Gold, R. Monoclonal Antibodies: The Evolution from '80s Magic Bullets To Mature, Mainstream Applications as Clinical Therapeutics. Genetic Engineering News Vol. 17, Number 14 (August 1997); Mendez M, Green L, Corvalan J, Jia X-C, Maynard-Currie C, Yang X-d, Gallo M, Louie D, Lee D, Erickson K, Luna J, Roy C, Abderrahim H, Kirschenbaum F, Noguchi M, Smith D, Fukushima A, Hales J, Finer M, Davis C, Zsebo K, Jakobovits A. Functional transplant of megabase human immunoglobulin loci recapitulates human antibody response in mice. Nature Genetics Vol. 15, pp: 146-156 (1997); Jakobovits A. Mice engineered with human immunoglobulin YACs: A new technology for production of fully human antibodies for autoimmunity therapy. Weir's Handbook of Experimental Immunology, The Integrated Immune System Vol. IV, pp: 194.1-194.7 (1996); Jakobovits A. Production of fully human antibodies by transgenic mice. Current Opinion in Biotechnology Vol. 6, No. 5, pp: 561-566 (1995); Mendez M, Abderrahim H, Noguchi M, David N, Hardy M, Green L, Tsuda H, Yoast S, Maynard-Currie C, Garza D, Gemmill R, Jakobovits A, Klapholz S. Analysis of the structural integrity of YACs comprising human immunoglobulin genes in yeast and in embryonic sGEM cells. Genomics Vol. 26, pp: 294-307 (1995); Jakobovits A. YAC Vectors: Humanizing the mouse genome. Current Biology Vol. 4, No. 8, pp: 761-763 (1994); Arbones M, Ord D, Ley K, Ratech H, Maynard-Curry K, Otten G, Capon D, Tedder T. Lymphocyte homing and leukocyte rolling and migration are impaired in L-selectin-deficient mice. Immunity Vol. 1, No. 4, pp: 247-260 (1994); Green L, Hardy M, Maynard-Curry K, Tsuda H, Louie D, Mendez M, Abderrahim H, Noguchi M, Smith D, Zeng Y, et. al. Antigen-specific human monoclonal antibodies from mice engineered with human Ig heavy and light chain YACs. Nature Genetics Vol. 7, No. 1, pp: 13-21 (1994); Jakobovits A, Moore A, Green L, Vergara G, Maynard-Curry K, Austin H, Klapholz S. Germ-line transmission and expression of a human-derived yeast artificial chromosome. Nature Vol. 362, No. 6417, pp: 255-258 (1993); Jakobovits A, Vergara G, Kennedy J, Hales J, McGuinness R, Casentini-Borocz D, Brenner D, Otten G. Analysis of homozygous mutant chimeric mice: deletion of the immunoglobulin heavy-chain joining region blocks B-cell development and antibody production. Proceedings of the National Academy of Sciences USA Vol. 90, No. 6, pp: 2551-2555 (1993); Kucherlapati et al., U.S. Pat. No. 6,1075,181.

Antibodies can also be made using phage display techniques. Such techniques can be used to isolate an initial antibody or to generate variants with altered specificity or avidity characteristics. Single chain Fv can also be used as is convenient. They can be made from vaccinated transgenic mice, if desired. Antibodies can be produced in cell culture, in phage, or in various animals, including but not limited to cows, rabbits, goats, mice, rats, hamsters, guinea pigs, sheep, dogs, cats, monkeys, chimpanzees, apes.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing antitumor agents (e.g., antisense oligonucleotides, plasmids which encode for toxins, methotrexate, etc.); and other antibodies or antibody fragments, such as F(ab).

Those of skill in the art will readily understand and be able to make such antibody derivatives, as they are well known in the art. The antibodies may be cytotoxic on their own, or they may be used to deliver cytotoxic agents to particular locations in the body. The antibodies can be administered to individuals in need thereof as a form of passive immunization.

Characterization of extracellular regions for the cell surface and secreted proteins from the protein sequence is based on the prediction of signal sequence, transmembrane domains and functional domains. Antibodies are preferably specifically immunoreactive with membrane associated proteins, particularly to extracellular domains of such proteins or to secreted proteins. Such targets are readily accessible to antibodies, which typically do not have access to the interior of cells or nuclei. However, in some applications, antibodies directed to intracellular proteins may be useful as well. Moreover, for diagnostic purposes, an intracellular protein may be an equally good target since cell lysates may be used rather than a whole cell assay.

Computer programs can be used to identify extracellular domains of proteins whose sequences are known. Such programs include SMART software (Schultz et al., Proc. Natl. Acad. Sci. USA 95: 5857-5864, 1998) and Pfam software (BaGEMan et al., Nucleic acids Res. 28: 263-266, 2000) as well as PSORTII. Typically such programs identify transmembrane domains; the extracellular domains are identified as immediately adjacent to the transmembrane domains. Prediction of extracellular regions and the signal cleavage sites are only approximate. It may have a margin of error + or −5 residues. Signal sequence can be predicted using three different methods (Nielsen et al, Protein Engineering 10:1-6, 1997, Jagla et. al, Bioinformatics 16: 245-250, 2000, Nakai, K. and Horton, P. Trends in Biochem. Sci. 24:34-35, 1999) for greater accuracy. Similarly transmembrane (TM) domains can be identified by multiple prediction methods. (Pasquier, et. al, Protein Eng. 12:381-385, 1999, Sonnhammer et al., In Proc. of Sixth Int. Conf. on Intelligent Systems for Molecular Biology, p. 175-182, Ed J. Glasgow, T. Littlejohn, F. Major, R. Lathrop, D. Sankoff, and C. Sensen Menlo Park, Calif.: AAAI Press, 1998, Klein, et. al, Biochim. Biophys. Acta, 815:468, 1985, Nakai and Kanehisa Genomics, 14: 897-911, 1992). In ambiguous cases, locations of functional domains in well characterized proteins are used as a guide to assign a cellular localization.

Putative functions or functional domains of novel proteins can be inferred from homologous regions in the database identified by BLAST searches (Altschul et. al. Nucleic Acid Res. 25: 3389-3402, 1997) and/or from a conserved domain database such as Pfam (BaGEMan et. al, Nucleic Acids Res. 27:260-262 1999) BLOCKS (Henikoff, et. al, Nucl. Acids Res. 28:228-230, 2000) and SMART (Ponting, et. al, Nucleic Acid Res. 27, 229-232, 1999). Extracellular domains include regions adjacent to a transmembrane domain in a single transmembrane domain protein (out-in or type I class). For multiple transmembrane domains proteins, the extracellular domain also includes those regions between two adjacent transmembrane domains (in-out and out-in). For type II transmembrane domain proteins, for which the N-terminal region is cytoplasmic, regions following the transmembrane domain is generally extracellular. Secreted proteins on the other hand do not have a transmembrane domain and hence the whole protein is considered as extracellular.

Membrane associated proteins can be engineered to delete the transmembrane domains, thus leaving the extracellular portions which can bind to ligands. Such soluble forms of transmembrane receptor proteins can be used to compete with natural forms for binding to ligand. Thus such soluble forms act as inhibitors. and can be used therapeutically as anti-angiogenic agents, as diagnostic tools for the quantification of natural ligands, and in assays for the identification of small molecules which modulate or mimic the activity of a GEM:ligand complex.

Alternatively, the endothelial markers themselves can be used as vaccines to raise an immune response in the vaccinated animal or human. For such uses, a protein, or immunogenic fragment of such protein, corresponding to the intracellular, extracellular or secreted GEM of interest is administered to a subject. The immogenic agent may be provided as a purified preparation or in an appropriately expressing cell. The administration may be direct, by the delivery of the immunogenic agent to the subject, or indirect, through the delivery of a nucleic acid encoding the immunogenic agent under conditions resulting in the expression of the immunogenic agent of interest in the subject. The GEM of interest may be delivered in an expressing cell, such as a purified population of glioma endothelial cells or a populations of fused glioma endothelial and dendritic cells. Nucleic acids encoding the GEM of interest may be delivered in a viral or non-viral delivery vector or vehicle. Non-human sequences encoding the human GEM of interest or other mammalian homolog can be used to induce the desired immunologic response in a human subject. For several of the GEMs of the present invention, mouse, rat or other ortholog sequences are described herein or can be obtained from the literature or using techniques well within the skill of the art.

Endothelial cells can be identified using the markers which are disclosed herein as being endothelial cell specific. These include the human markers identified by SEQ ID NOS: 1-510. Antibodies specific for such markers can be used to identify such cells, by contacting the antibodies with a population of cells containing some endothelial cells. The presence of cross-reactive material with the antibodies identifies particular cells as endothelial. Similarly, lysates of cells can be tested for the presence of cross-reactive material. Any known format or technique for detecting cross-reactive material can be used including, immunoblots, radioimmunoassay, ELISA, immunoprecipitation, and immunohistochemistry. In addition, nucleic acid probes for these markers can also be used to identify endothelial cells. Any hybridization technique known in the art including Northern blotting, RT-PCR, microarray hybridization, and in situ hybridization can be used.

One can identify glioma endothelial cells for diagnostic purposes, testing cells suspected of containing one or more GEMs. One can test both tissues and bodily fluids of a subject. For example, one can test a patient's blood for evidence of intracellular and membrane associated GEMs, as well as for secreted GEMs. Intracellular and/or membrane associated GEMs may be present in bodily fluids as the result of high levels of expression of these factors and/or through lysis of cells expressing the GEMs.

Populations of various types of endothelial cells can also be made using the antibodies to endothelial markers of the invention. The antibodies can be used to purify cell populations according to any technique known in the art, including but not limited to fluorescence activated cell sorting. Such techniques permit the isolation of populations which are at least 50, 60, 70, 80, 90, 92, 94, 95, 96, 97, 98, and even 99% the type of endothelial cell desired, whether normal, tumor, or pan-endothelial. Antibodies can be used to both positively select and negatively select such populations. Preferably at least 1, 5, 10, 15, 20, or 25 of the appropriate markers are expressed by the endothelial cell population.

Populations of endothelial cells made as described herein, can be used for screening drugs to identify those suitable for inhibiting the growth of tumors by virtue of inhibiting the growth of the tumor vasculature.

Populations of endothelial cells made as described herein, can be used for screening candidate drugs to identify those suitable for modulating angiogenesis, such as for inhibiting the growth of tumors by virtue of inhibiting the growth of endothelial cells, such as inhibiting the growth of the tumor or other undesired vasculature, or alternatively, to promote the growth of endothelial cells and thus stimulate the growth of new or additional large vessel or microvasculature.

Inhibiting the growth of endothelial cells means either regression of vasculature which is already present, or the slowing or the absence of the development of new vascularization in a treated system as compared with a control system. By stimulating the growth of endothelial cells, one can influence development of new (neovascularization) or additional vasculature development (revascularization). A variety of model screening systems are available in which to test the angiogenic and/or anti-angiogenic properties of a given candidate drug. Typical tests involve assays measuring the endothelial cell response, such as proliferation, migration, differentiation and/or intracellular interaction of a given candidate drug. By such tests, one can study the signals and effects of the test stimuli. Some common screens involve measurement of the inhibition of heparanase, endothelial tube formation on Matrigel, scratch induced motility of endothelial cells, platelet-derived growth factor driven proliferation of vascular smooth muscle cells, and the rat aortic ring assay (which provides an advantage of capillary formation rather than just one cell type).

Drugs can be screened for the ability to mimic or modulate, inhibit or stimulate, growth of tumor endothelium cells and/or normal endothelial cells. Drugs can be screened for the ability to inhibit tumor endothelium growth but not normal endothelium growth or survival. Similarly, human cell populations, such as normal endothelium populations or glioma endothelial cell populations, can be contacted with test substances and the expression of glioma endothelial markers and/or normal endothelial markers determined. Test substances which decrease the expression of glioma endothelial markers (GEMs) are candidates for inhibiting angiogenesis and the growth of tumors. In cases where the activity of a GEM is known, agents can be screened for their ability to decrease or increase the activity.

For those glioma endothelial markers identified as containing transmembrane regions, it is desirable to identify drug candidates capable of binding to the GEM receptors found at the cell surface. For some applications, the identification of drug candidates capable of blocking the GEM receptor from its native ligand will be desired. For some applications, the identification of a drug candidate capable of binding to the GEM receptor may be used as a means to deliver a therapeutic or diagnostic agent. For other applications, the identification of drug candidates capable of mimicking the activity of the native ligand will be desired. Thus, by manipulating the binding of a transmembrane GEM receptor:ligand complex, one may be able to promote or inhibit further development of endothelial cells and hence, vascularization.

For those glioma endothelial markers identified as being secreted proteins, it is desirable to identify drug candidates capable of binding to the secreted GEM protein. For some applications, the identification of drug candidates capable of interfering with the binding of the secreted GEM it is native receptor. For other applications, the identification of drug candidates capable of mimicking the activity of the native receptor will be desired. Thus, by manipulating the binding of the secreted GEM:receptor complex, one may be able to promote or inhibit further development of endothelial cells, and hence, vascularization.

Expression can be monitored according to any convenient method. Protein or mRNA can be monitored. Any technique known in the art for monitoring specific genes' expression can be used, including but not limited to ELISAs, SAGE, microarray hybridization, Western blots. Changes in expression of a single marker may be used as a criterion for significant effect as a potential pro-angiogenic, anti-angiogenic or anti-tumor agent. However, it also may be desirable to screen for test substances which are able to modulate the expression of at least 5, 10, 15, or 20 of the relevant markers, such as the tumor or normal endothelial markers. Inhibition of GEM protein activity can also be used as a drug screen. Human and mouse GEMS can be used for this purpose.

Test substances for screening can come from any source. They can be libraries of natural products, combinatorial chemical libraries, biological products made by recombinant libraries, etc. The source of the test substances is not critical to the invention. The present invention provides means for screening compounds and compositions which may previously have been overlooked in other screening schemes. Nucleic acids and the corresponding encoded proteins of the markers of the present invention can be used therapeutically in a variety of modes. GEMs can be used to stimulate the growth of vasculature, such as for wound healing or to circumvent a blocked vessel. The nucleic acids and encoded proteins can be administered by any means known in the art. Such methods include, using liposomes, nanospheres, viral vectors, non-viral vectors comprising polycations, etc. Suitable viral vectors include adenovirus, retroviruses, and sindbis virus. Administration modes can be any known in the art, including parenteral, intravenous, intramuscular, intraperitoneal, topical, intranasal, intrarectal, intrabronchial, etc.

Specific biological antagonists of GEMs can also be used to therapeutic benefit. For example, antibodies, T cells specific for a GEM, antisense to a GEM, and ribozymes specific for a GEM can be used to restrict, inhibit, reduce, and/or diminish tumor or other abnormal or undesirable vasculature growth. Such antagonists can be administered as is known in the art for these classes of antagonists generally. Anti-angiogenic drugs and agents can be used to inhibit tumor growth, as well as to treat diabetic retinopathy, rheumatoid arthritis, psoriasis, polycystic kidney disease (PKD), and other diseases requiring angiogenesis for their pathologies.

Mouse counterparts to human GEMS can be used in mouse cancer models or in cell lines or in vitro to evaluate potential anti-angiogenic or anti-tumor compounds or therapies. Their expression can be monitored as an indication of effect. Mouse GEMs can be used as antigens for raising antibodies which can be tested in mouse tumor models. Mouse GEMs with transmembrane domains are particularly preferred for this purpose. Mouse GEMs can also be used as vaccines to raise an immunological response in a human to the human ortholog.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### Example 1

In this study we employ SAGE transcript profiling to derive the transcriptomes from normal and neoplastic brain tissue. Moreover, we have employed a new version of SAGE, long SAGE, allowing for the derivation of 21 bp SAGE tags. These longer tags allow for the direct interrogation of genomic DNA, identifying unique locations of cell-specific transcription. Endothelial cells from normal brain and different stages of gliomas were expression profiled and compared to each other and to the colon endothelial cell data. Distinct sets of genes define global tumor and normal endothelial cell markers as well as defining glioma-specific endothelial markers. This expanded tumor endothelial cell database will likely provide further insights into the complex regulatory mechanisms governing tumor angiogenesis.

### Example 2

Tissue procurement and endothelial cell isolation. Five separate brain tissue samples (Table 1) were resected and immediately subjected to endothelial cell isolation with slight modifications to the protocol described previously. St Croix, B., Rago, C., Velculescu, V., Traverso, G., Romans, K. E., Montgomery, E., Lal, A., Riggins, G. J., Lengauer, C., Vogelstein, B., and Kinzler, K. W. (2000). Genes expressed in human tumor endothelium. Science 289, 1197-202.

Briefly, samples were surgically excised and submerged in DMEM. The samples were minced into 2 centimeter cubes and subjected to tissue digestion with a collagenase cocktail. Samples were mixed at 37° C. until dissolved. Cells were spun down and washed two times with PBS/BSA and filtered through successive nylon mesh filters of 250, 100 and 40 microns. Samples were resuspended in PBS/BSA and applied to a 30% Percoll gradient centrifuging for 15 minutes at 800 g. 5 ml off the top of the percoll gradient was diluted in 50 ml DMEM and cells pelleted, washed with PBS and resuspended in 3 ml PBS/BSA. Cells were filtered through falcon blue top filter tubes, spun down and resuspended in 1 ml PBS/BSA. 100 microliters of prewashed ant-CD45 magnetic beads (Dynal) were added and the solution allowed to gently mix for ten minutes. Bead-bound cells were discarded and the supernatant transferred to a fresh microcentrifuge tube. 10 microliters of P1H12 mAB (1:100) (Brain N1, T1, and T2 samples) or UEA-I lectin (Brain N2 and T3 samples) was added and the samples were mixed gently at 4° C. for 45 minutes. Cells were pelleted and washed 3 times in PBS/BSA and resuspended in 500 microliters PBS/BSA. Prewashed goat anti-mouse M450 dynabeads were added to each tube and allowed to mix for 15 minutes at 4° C. Bead-bound cells were washed 8 times with PBS/BSA and resuspended in a final volume of 500 microliters PBS. Cells were counted and frozen at −70° C. prior to RNA extraction.

### Example 3

RNA isolation and SAGE library generation. RNA was isolated from the selected cells and initially subjected to RT-PCR analysis to determine the relative abundance of specific, known endothelial cell markers. The microSAGE protocol St Croix, B., Rago, C., Velculescu, V., Traverso, G., Romans, K. E., Montgomery, E., Lal, A., Riggins, G. J., Lengauer, C., Vogelstein, B., and Kinzler, K. W. (2000). Genes expressed in human tumor endothelium. Science 289, 1197-202 (server www, domain name sagenet.org, directory sage protocol) was used to generate high-quality longSAGE libraries employing the tagging enzyme MmeI instead of BsmFI. 21 base tags were defined by capillary sequencing using a combination of an ABI 3700 and ABI 3100. The sample descriptions and sequencing depth are shown in Table 3.

### Example 4

Data analysis. Long SAGE tags derived from the brain endothelial samples were reduced to short tags to allow for the integration of colon endothelial SAGE data. Aggregate short tags were derived from the long tags. Any short tag counts that had more than one corresponding long tag representative were summed and the counts represented as one short tag. Both sequencing errors and legitimate long tag derivatives contribute to the generation of multiple long tags. For transcript and genome mapping, differential long tags were employed. Differential gene expression was evaluated as follows: For the two normal brain samples, either the maximum or minimum value was used for determining tumor/normal and normal/tumor ratios, respectively. For the three brain tumor samples, the median value was used for the tumor/normal whereas the maximum value was used for the normal/tumor ratios. A two parameter family of beta distributions was used to assess the probability of observing two fold differences in the observed SAGE tag abundances. Chen, H., Centola, M., Altschul, S. F., and Metzger, H. (1998). Characterization of gene expression in resting and activated mast cells. J Exp Med 188, 1657-68.

### Example 5

The following provides a detailed protocol useful for isolating brain endothelial cells. All steps were done at 4° C. in cold room and in centrifuge except digestion.
1) Take sample from operating room and submerge in known volume of DMEM+ in 50 ml conical tube to measure tumor volume by displacement. Cut off 2 small pieces of tumor on dry ice and store at −70° C. for mRNA extraction/immunohistochemistry/in situ analysis.
2) Take sample from conical and place in small amount of DMEM+ in 10 cm Tissue Culture dish in hood. Mince specimen into 2 mm cubes with sterile scalpel.
3) Transfer minced specimen to small autoclaved erlenmeyer flask and add 5× volume of digestion cocktail. Sample volumes >5 ml should be split into multiple flasks.
4) Mix in bacterial shaker or in 37° C. room on rotating shaker for 45 minutes or until sample is dissolved. Titrate with 10 ml piper every 15 minutes. Once a good cell suspension is obtained, remove and transfer to 50 ml conical.
   Remainder of protocol done at 4° C.
5) Spin down at 1500 RPM (600×g) at 4° C. for 5 minutes.
6) Wash 2× with PBS/BSA and spin down again. Pool samples.
7) Filter through Nylon Mesh (250, 100, 40 micron).
8) Spin down.
9) Resuspend n PBS/BSA at ½ the original tumor volume.
10) Apply sample in 500 ul aliquots to preformed 30% Percoll gradient (Gradients needed=volume of original sample).
11) Spin at 1750 RPM (800 g) for 15 minutes.
12) Remove top 5 ml Percoll from each tube and dilute with DMEM to 50 ml volume.
13) Pellet cells in centrifuge at 1500 RPM. Pool pelleted cells.
14) Wash 2× with PBS/BSA and resuspend in 3 ml PBS/BSA.
15) Filter through Falcon Blue Top Filter tube.
16) Spin down and resuspend in 1 ml PBS/BSA in a 1.5 ml microcentrigufe tube.
17) Add 100 μl of prewashed anti-CD45 beads (hematopoietic depletion) to solution and rotate end over end in cold room for ten minutes. [For brain tissue isolation, an additional negative selection with BerEP4 epithelial depletion is not needed]
18) Remove bead-bound cells and transfer supernatant to a fresh microcentrifuge tube. Save bead-bound sample by freezing at −70° C. Repeat extraction to ensure complete removal of all beads.
19) Add 10 ul of P1H12 mAb (1:100) to cells and mix in cold room with end-over-end rotation for 45 minutes. [As an alternative, selection using UEA1 lectin also provides quality endothelial cell selection.]
20) Pellet cells and wash 3× with PBS/BSA.
21) Resuspend cells in 500 ul of PBS/BSA.
22) Divide sample into four 1.5 ml microcentrifuge tubes (125 ul per tube) and bring volume up to 800 ul. Add 50 ul of prewashed goat anti-mouse M450 dynabeads to each tube.
23) Rotate tubes in cold room for 15 minutes.
24) Separate with magnet and save supernatant as staining control, tumor/brain fraction.
25) Rinse 8× with PBS/BSA.
26) Pool beads into single microcentrifuge tube.
27) Resuspend final cells in 500 ul plain PBS.
28) Take 5 ul of solution and combine with 5 ul of Magic DAPI and count on hemacytometer.
29) Remove 10 k cells for staining for quality control based on hemacytometer results
30) Separate beads again and freeze remainder at −70° C. for mRNA extraction.

### Example 6

This example describes the preparation of SAGE tags from mRNA extracted from brain endothelial cells. The preparation is described with reference to standard SAGE tag preparation procedures as are known in the art.
All of the template was used in the PCR SAGE ditag step. Usually we take only a small portion of our template, dilute it and perform ˜300 PCR reactions. For these libraries we used all of our material, diluted it and performed ˜1200 PCR reactions.
   During the post-amplified PCR product purification step we normally do a standard large volume phenol/chloroform extraction and remove the aqueous layer which contains the product of interest. For these libraries we used Eppendorfs Phase Lock product which creates a physical barrier between the aqueous and organic layers thereby decreasing the amount of product you leave behind. This product was used for all P/C extractions in the second half of the protocol.
   Digesting the amplified PCR products with NlaIII to release the ditag of interest is usually done in one reaction. For these libraries I divided the material into thirds and performed 3 NlaIII reactions in the hopes of yielding more released ditag.
   Due to the low amount of material, upon entering the concatemer and digested pZERO ligation reaction, I modified the recipe for this reaction to accommodate this. Standard reaction calls for 6 ul of concatemers, 2 ul of 5× ligase buffer, 1 ul digested pZERO vector, and 1 ul of high concentrate ligase. I modified it to 6 ul of concatemers, 2 ul of 5× ligase buffer, 0.3-0.5 ul of digested pZERO vector, 1 ul high concentrate ligase and filled the missing volume with water. My intention was to favor the concatemer to pZERO ligation reaction relative to the competing pZERO to pZERO ligation reaction.
Most gels during the procedure showed weak amounts of product for visualization and the concatemer gels showed no visible product via the naked eye (we cut out certain fractions regardless).

### Example 7

Microarray Analysis. Custom 50 nucleotide oligomer arrays were constructed containing 606 unique gene elements. The 606 genes were derived from tumor and normal induced genes from both colon and brain data (328 genes), as well as 278 genes from both literature reviews and housekeeping genes. Arrays were interrogated with Cy3 and Cy5 dye-swapped labelled aRNA samples comparing HMVECs grown on plastic, collagen, fibrin, or Matrigel.

### Example 8

In situ Hybridizations and Immunohistochemistry. In situ hybridizations for PV I, VEGFR2 and vWF were carried out as described previously (10). Co-staining of PV1 and CD31 was carried out as follows: Four 500 nucleotide riboprobe fragments specific for PV1 were transcribed and used to probe formalin fixed 5 micron tissue sections. Final detection of the bound riboprobes were delayed until after the CD31 IHC staining. After PV1 hybridization and washing, tissue sections were fixed for 20 minutes in 4% formaldehyde. After a brief rinse in TBS, antigen retrieval was carried out using DAKO target retrieval solution (DAKO, Cat#S 1699) according to manufacturer's instructions. After a five minute wash in TBS, slides were digested with Proteinase K at 20 ng/ml in TBS for 20 minutes at 37T, then blocked for 20 minutes at room temperature in block (10% Goat serum/0.5% Casein/0.05% Tween-20/PBS). Slides were incubated with DAKO CD31 (Cat#M0823) at a final concentration of 1 microgram/slide in block solution, for 60 minutes at room temperature. After two 5 minute TBST (DAKO, Cat#S3306) washes at room temperature, PV1 riboprobe and CD31 antibody were detected with Streptavidin-Cy2 (Jackson ImmunoResearch, Cat#016-220-084) at 5 micrograms/slide for the PV1 riboprobe, and goat anti-mouse-Cy3 (Jackson ImmunoResearch, Cat#115-165-146) at 2.5 micrograms/slide for CD31, for 60 minutes at room temperature. After three Ywashes in TBST, the slides were mounted with antifade medium containing DAPI nuclear counter-stain, cover-slipped and stored at −20′C until viewing. Single images of DAPI, Cy2 and Cy3 images were acquired separately on a Zeiss Axioplan at 40× with a Hammamatsu camera, then merged together to form a composite image using universal imaging metamorph software, and stored at −20C until viewing.

### Example 9

Capillary-like tubule formation assay. The formation of capillary-like tubular structures was assessed in Matrigel-coated multiwell plates essentially as described previously (12). Briefly, 300 microliters of Matrigel (BD, Bedford, Mass.) was added to each well of a 24 well plate and allowed to polymerize at 37″C for 30 minutes. HMVECs (BioWhittaker) were infected with adenovius harboring Tem.1 or GFP gene or empty vector (EV) for 67 hours at 300 MOI (Multiplicity Of Infection). Cells were then seeded at a density of 30×10³ cells/well in 500 microliters EGM-2 medium with supplements (BioWhittaker) in Matrigel-coated plates and incubated at 37′C for 24 hours and viewed using a Nikon Eclipse TE200 microscope under a phase contrast and photographed. Images were analyzed using software Scion Image (Scion Corporation, Frederick, Md.) under the mode of integrated density.

### Example 9

Cell Proliferation Assay. HMVEC proliferation was assessed by the Cell Titer-Glo Luminescent Cell Viability Assay (Promega, Madison, Wis.) in 96-well cell culture plates. HMVECs were seeded at 2,000 cells per well in 100 microliters medium and plates were incubated at 37′C for 48 hours. Reagent was added to each well according to manufacture's instruction, and fluorescence was measured using the Millipore CytoFluor2350.

### Example 10

Five independent endothelial cell populations were purified from glioma tumor tissue and normal brain tissue. In this study, the tissue defined as normal is derived from patients with epilepsy who have undergone a temporal lobectomy. The samples are summarized in Table 3. Samples N1, T1 and T2 were ultimately P1H12-selected and samples N2 and T3 were UEA-I selected. Prior to SAGE analysis, each sample was assessed for the relative mRNA abundance for vWF, Glial fibrillary acidic protein (GFAP) and EF1 by RT-PCR. Abundant levels of vWF and the control housekeeper EF1, and low levels of the glial cell-specific gene GFAP suggested the cell population was primarily endothelial (data not shown). SAGE analysis was performed to a depth of approximately 50,000 tags (Table 3). For data analysis, each SAGE project was normalized to exactly 50,000 tags. Pairwise comparisons between expression data derived from tumor samples selected with P1H12 or UEA-I showed correlation coefficients around 80%, slightly higher than a comparison between two tumor samples both selected with P1H12. This suggests that selecting endothelial cells with either P1H12 or UEA-I results in highly similar cell populations. Moreover, nearly half of the tumor specific markers revealed in this study are induced 4 fold in each of the normal samples used, suggesting the normal samples are similar populations as well. With this in mind, we felt that combining data for the two normal samples and for the three tumor samples was appropriate.

TABLE 3
Samples used in this study.

| | | Tags | EC |
|---|---|---|---|
| Sample | Description | Generated | Selection |
| Brain N1 | Normal temporal lobectomy ECs | 43,000 | P1H12 |
| Brain N2 | Normal temporal lobectomy ECs | 49,000 | UEA-I |
| Brain T1 | Grade IV Glioma Ecs | 46,000 | P1H12 |
| Brain T2 | Grade III Glioma Ecs | 50,000 | P1H12 |
| Brain T3 | Grade IV Glioma Ecs | 58,000 | UEA-I |
| Colon N* | Normal colon Ecs | 96,000 | P1H12 |
| Colon T* | Tumor colon Ecs | 96,000 | P1H12 |
| Fetal Brain | Normal bulk | 204,000 | — |
| Fetal Kidney | Normal bulk | 50,000+ | — |

Genes specific for endothelial cells showed expression levels consistent with the previously examined colon endothelial SAGE data (Table 4). Additionally, markers specific for epithelial, hematopoeitic or glial cells showed limited or no expression in the brain endothelial libraries suggesting little contamination from non-endothelial cell populations (Table 4). Finally, the data generated here allow for the derivation of alt gene EC prediction class of which 6 have been previously described as EC-specific (Huminiecki, L., and Bicknell, R. (2000). In silico cloning of novel endothelial-specific genes. Genome Res 10, 1796-806.) (data not shown). This provides further evidence of pure EC populations used for this study.

TABLE 4
Cell specificity markers.

| | | Co- | Co- | | | | | |
|---|---|---|---|---|---|---|---|---|
| | Speci- | lon | lon | Brain | Brain | Brain | Brain | Brain |
| Gene | ficity | N | T | N1 | N2 | T1 | T2 | T3 |
| Hevin | EC | 161 | 69 | 51 | 99 | 223 | 121 | 48 |
| VWF | EC | 35 | 33 | 12 | 53 | 37 | 51 | 110 |
| Tie2/Tek | EC | 4 | 2 | 2 | 4 | 1 | 4 | 3 |
| CD34 | EC | 5 | 2 | 3 | 10 | 12 | 4 | 11 |
| CD14 | Hemato- | 1 | 1 | 1 | 2 | 0 | 0 | 1 |
| | poeitic | | | | | | | |
| CK8 | Epithelial | 1 | 2 | 0 | 0 | 2 | 1 | 1 |
| GLUT1 | Brain EC | 0 | 1 | 8 | 37 | 2 | 25 | 8 |
| GFAP | Glial | 0 | 0 | 0 | 0 | 0 | 0 | 0 |

Genes expressed preferentially in glioma derived endothelial cells as opposed to normal endothelial cells are potentially involved in regulating angiogenesis-dependent tumor growth. Specific parameters for the sorting of SAGE data and the layering of additional statistical filters allowed for a conservative estimate of legitimate differentially expressed genes (see Methods). Excluding mitochondrial genes, 131 genes were observed to be induced in the glioma endothelial cells based on a four fold induction ratio. Only 14 genes can be entertained as glioma-specific when additional statistical filters are applied (Table 5). In this case, a two fold parameter family of distributions was used to establish a 90% probability of observing at least a 2 fold difference in values. Only one of these twelve genes, apolipoprotein D, shows higher expression in the stage III glioma than at least one of the stage IV tumors. This suggests that many of the highly induced glioma endothelial genes revealed in this analysis may be involved in later stages of angiogenesis where the initiation of vascular sprouting has already occurred or are glioma type specific showing representation in the astrocytoma and not oligodendroglioma-derived ECs. Less highly induced genes, or genes primarily induced in the less aggressive tumor stage, may be more reflective of angiogenesis initiation. Several genes regulating extracellular matrix architecture are revealed as highly induced in this study. HSPG2 (perlecan), several type IV collagen transcript variants, and matrix metalloprotease 14 (MMP14) have all been shown to play a role in remodeling the extracellular matrix. Interestingly, other genes that play roles in either cellular signaling or cell-cell communication are also highly expressed exclusively in glioma-associated endothelial cells. Melanoma associated antigen (MG50), endothelin receptor, the G-protein coupled receptor RDC-1, and integrin αV are all cell surface proteins previously demonstrated to play a role in signaling cascades. Although the endothelin receptor, RDC-1 and integrin αV have previously been shown to regulate angiogenesis, MG50 does not have an association with angiogenesis. Moreover, MG50 was previously shown to be selectively associated with several types of tumor cells with a function yet to be defined. It is noteworthy that the p53-induced, brain-specific angiogenesis inhibitor (BAI-1) was expressed to significant levels but restricted to the earlier stage tumor present in this study (data not shown). It is possible that the loss of expression of BAI-1 in the later tumor stages reflects the need to more aggressively advance vascular development. Other than the detection of a differential HEYL SAGE tag, no other colon endothelial markers were observed to be preferentially expressed in the grade III tumor. In total, of the 14 tumor induced genes listed, 12 are either present on the cell surface or secreted. The localization of the remaining two gene products has yet to be determined as these genes remain uncharacterized. Finally, it is noteworthy that only a select few genes show significant (>2 tags) expression in a fetal brain library where angiogenesis is expected to be robust.

In contrast to the highly biased localization of glioma-induced endothelial cell gene expression defined above, genes that are induced in the normal endothelial cells relative to glioma endothelial cells show a radically different cellular distribution. Twenty-one genes are induced 4 fold or greater in the normal endothelial cells. Filtering for genes with a 50% or greater chance of having greater than 2 fold difference in transcript abundance reduces this list to 14 genes (Table 6). Protein products predicted for these 14 genes show a range of cellular localizations with 4 gene products being intracellular, 5 being integral membrane proteins, 3 extracellular, and one each either secreted, on the cell surface or a nuclear membrane receptor. Several of these genes have functions consistent with either tumor suppressor or anti-angiogenic functions. These anti-proliferative functions have been ascribed to the early growth response gene 1 (EGR1), BTG2, Fruppel-like factor 4 (KLF4), and the serine protease inhibitor SPINT2 although associations with angiogenesis are limited to SPINT2. The down-regulation of these genes in each of the three glioma tumors suggests that these genes may function to encode proteins with anti-angiogenic properties. Both SPINT2 and BTG2 are secreted and may act via paracrine mechanisms. Also noteworthy is the preferential expression of the secreted protein MT1A as this metalothionein may serve as an antioxidant potentially attenuating DNA damage within adjacent cells. Interestingly, EGR1 and KLF4′ encode transcription factors suggesting that some part of the anti-angiogenic pathway revealed here may be initiated by these gene products. With the exception of MT1A, none of the above genes show differential expression in colon tumor ECs and may therefore be glioma-specific EC markers.

The specificity of gene expression for tumor EC subtypes is important to define and can be addressed with the glioma EC data integrated with data obtained previously for colon EC populations. A limited number of genes are preferentially expressed in both brain and colon normal EC populations. In contrast, 16 genes were induced at least 4 fold in both colon and brain tumor EC fractions. 12 of these genes also met the criteria of having a greater than 50% chance of being at least 2 fold differential (Table 7). The majority of these genes (7) are collagen transcripts. However, tumor endothalial marker 1 (TEM1), THY1, and RDC-1 also show consistent induction in the different tumor EC cells. This limited conservation of tumor-induced EC expression suggests highly specific EC expression profiles dependent on the tissue source. TEM1 expression has been validated on tissue arrays harboring tissue slices from astrocytomas (data not shown).

Defining the specificity of gene expression to particular cell types can assist in determining function and designing therapeutics. Our non-endothelial cell SAGE database currently contains 76 libraries encoding 255,000 unique SAGE transcripts. The epithelial cell lines derive from lung, ovary, kidney, prostate, breast, colon, pancreas. Additional non-epithelial sources include cardiomyocytes, melanocytes, glioblastoma and monocytes. Genes which show induction in glioma ECs and demonstrate a restricted expression in non-EC cells may be ideal targets for anti-angiogenic therapies. Allowing for 1 or fewer tags in any non-EC library and at least a four-fold induction in glioma ECs yielded only 5 genes (Table 8). Some of these genes are likely not EC-specific due to the relatively limited number of cell types included within the non-EC database. However, both PV-1 and Plexin A2 (PLXNA2) are interesting genes with potential functional relevance to angiogenesis regulation.

The SAGE tag that defines PLXNA2 falls outside of the current mRNA boundaries residing 3′ of the ultimate exon. RT-PCR results, however, have confirmed transcription of mRNA containing this tag in the tumor samples used to derive the SAGE data. Plexins share homology with the scatter factor/hepatocyte growth factor (SF/HGF) family of receptors encoded by the MET gene family [Tamagnone, L., Artigiani, S., Chen, H., He, Z., Ming, G. I., Song, H., Chedotal, A., Winberg, M. L., Goodman, C. S., Poo, M., Tessier-Lavigne, M., and Comoglio, P. M. (1999). Plexins are a large family of receptors for transmembrane, secreted, and GPI-anchored semaphorins in vertebrates. Cell 99, 71-80.] Earlier results have demonstrated a link between SF/HGF expression and increase tumorigencity [Bowers, D. C., Fan, S., Walter, K. A., Abounader, R., Williams, J. A., Rosen, E. M., and Laterra, J. (2000). Scatter factor/hepatocyte growth factor protects against cytotoxic death in human glioblastoma via phosphatidylinositol 3-kinase- and AKT-dependent pathways. Cancer Res 60, 4277-83.] Moreover, SF/HGF promotes this increased tumorigencity with concordant stimulation in angiogenesis [Lamszus, K., Laterra, J., Westphal, M., and Rosen, E. M. (1999). Scatter factor/hepatocyte growth factor (SF/HGF) content and function in human gliomas. Int J Dev Neurosci 17, 517-30.] In vivo targeting of SF/HGF was demonstrated to inhibit glioma growth and angiogenesis [Abounader, R., Lal, B., Luddy, C., Koe, G., Davidson, B., Rosen, E. M., and Laterra, J. (2002). In vivo targeting of SF/HGF and c-met expression via U1snRNA/ribozymes inhibits glioma growth and angiogenesis and promotes apoptosis. Faseb J 16, 108-10.]. Plexins are known to function as coreceptors with neuropilin 1 functioning as a receptor for semaphorin and, in turn, regulating neuronal guidance and cell association [Tamagnone, 1999, supra]. As neuropilin-1 and Plexin association can serve to receive signals from semaphorins to guide neuronal growth, it is conceivable that a Plexin-neuropilin association may regulate angiogenic growth in a manner analogous to KDR-neuropilin complexes signaling VEGF responses. Plexin A2 shows very low level expression in colon ECs and is not differentially induced in colon tumor ECs. It is noteoworthy that another plexin, plexin B2 (PLXNB2), also showed a five fold increase in glioma EC expression but did not make the statistical threshold demanded for Table 8. Plexin B2 was previously shown to be differentially induced in brain tumors [Shinoura, N., Shamraj, O. I., Hugenholz, H., Zhu, J. G., McBlack, P., Warnick, R., Tew, J. J., Wani, M. A., and Menon, A. G. (1995). Identification and partial sequence of a cDNA that is differentially expressed in human brain tumors. Cancer Lett 89, 215-21.] The upregulation of plexins in glioma ECs allows for a hypothesis whereby SF/HGF directly stimulates EC migration and proliferation. The novel discovery of a consistently upregulated level of Plexin A2 in gliomas requires further evidence for a functional link between tumor levels of plexin A2 and angiogenesis regulation, particularly in the brain.

PV-1 (also called PLVAP for plasmallema vesicle associated protein), is a recently discovered type II integral membrane glycoprotein shown to colocalize with caveolin-1. Stan, R. V., Arden, K. C., and Palade, G. E. (2001). cDNA and protein sequence, genomic organization, and analysis of cis regulatory elements of mouse and human PLVAP genes. Genomics 72, 304-13. Interestingly, this protein was the first to be shown to localize to the stomatal diaphragms and transendothelial channels within caveolae. The specific function of PV-1 remains unknown. PV-1 is expressed at substantial levels in colon ECs but is not expressed differentially between normal and tumor colon ECs. The upregulation of this caveolae-associated protein in gliomas may provide a means for specifically targeting glioma-associated endothelial cells as well as potentially providing a therapeutic delivery mechanism to the underlying tumorigenic cells (Marx, J. (2001). Caveolae: a once-elusive structure gets some respect. Science 294, 1862-5.))

From this study there is also the potential to define brain EC specific genes irrespective of function or differential expression in normal or tumor tissue. Applying the same criteria as that applied for defining EC restricted glioma induced genes, only two genes, TNFα-induced protein 3 and JUNB, show consistent expression in the brain EC samples but severely limited expression in non-EC databases.

The blood brain barrier within brain capillary endothelial cells results in a restricted diffusion of both small and large molecules as compared to non-brain EC junction complexes. As a result of this, brain capillary ECs facilitate molecular exchange via a tightly regulated, or catalyzed transport system. Any differential expression of catalyzed membrane transporters between normal and tumor tissue may provide a means to selectively deliver therapies to tumor cells. The insulin receptor (IR) has been known for some time to be a marker for brain capillary ECs and to facilitate delivery of drugs. One of the most highly induced, glioma-specific genes in this study is the IR (Table 8). The high induction of IR transcripts in gliomas was not previously recognized and may provide a selective delivery mechanism to cancer cells as these receptors are also proposed to reside within caveolae structures [Smith, R. M., Jarret, L. (1988). Lab. Invest. 58, 613-629.] Overall, very few transporters showed a differential induction in glioma-associated ECs as compared to their normal counterpart (Table 9). This is counter to previous suggestions linking altered expression of transporters with histologic grade of CNS tumors [Guerin, C., Wolff, J. E., Laterra, J., Drewes, L. R., Brem, H., and Goldstein, G. W. (1992). Vascular differentiation and glucose transporter expression in rat gliomas: effects of steroids. Ann Neurol 31, 481-7.] Only one other gene, SLC1A5 Solute carrier family 1 member 5 (neutral amino acid transporter), showed a greater than 4 fold induction in glioma-derived ECs. It should be stated, however, that the standard SAGE tag for integrin αV is shared with aquaporin. Long tag derivations of these two genes revealed that both integrin αV and aquaporin are induced in glioma ECs. Aquaporin may play a role in caveolae swelling that accompanies VEGF stimulated EC growth [Roberts, W. G., and Palade, G. E. (1997). Neovasculature induced by vascular endothelial growth factor is fenestrated. Cancer Res 57, 765-72.] Only one membrane transporter, Na+/K+ transporting ATP1A2 ATPase, was reciprocally repressed in glioma-derived ECs. It remains possible that certain transporters were missed in this analysis due to incorrect functional assignment. Nonetheless, the low number of differentially regulated transport facilitators suggests a small number of these genes need to be transcriptionally activated to accommodate any necessary increase in protein abundance required for tumor growth.

Table 10 shows genes induced in glioma endothelial cells but not in colon tumor or breast tumor endothelial cells.

Table 11 shows genes which encode transporters which are repressed in glioma endothelial cells.

Table 12 shows genes which encode proteins which are localized to the nucleus of both brain and colon tumor endothelial cells.

Table 13 shows genes which encode proteins which are localized to the cytoplasm of both brain and colon tumor endothelial cells.

Table 14 shows genes which encode proteins which are extracellular from both brain and colon tumor endothelial cells.

Table 15 shows genes which encode proteins which are localized to the membrane of both brain and colon tumor endothelial cells.

Table 16 shows genes which encode proteins which are induced in both brain and colon tumor endothelial cells.

Table 17 shows additional tumor endothelial markers in brain.

Table 18 shows tumor endothelial markers in the brain which are cytoplasmic.

Table 19 shows tumor endothelial markers in the brain which are nuclear.

Table 20 shows tumor endothelial markers in the brain which are membrane associated.

Table 21 shows tumor endothelial markers in the brain which are extracellular.

Table 22 shows tumor endothelial markers in the brain which are unsorted with respect to cellular localization.

### REFERENCES

Abounader, R., Lal, B., Luddy, C., Koe, G., Davidson, B., Rosen, E. M., and Laterra, J. (2002). In vivo targeting of SF/HGF and c-met expression via U1snRNA/ribozymes inhibits glioma growth and angiogenesis and promotes apoptosis. Faseb J 16, 108-10.
Bart, J., Groen, H. J., Hendrikse, N. H., van der Graaf, W. T., Vaalburg, W., and de Vries, E. G. (2000). The blood-brain barrier and oncology: new insights into function and modulation. Cancer Treat Rev 26, 449-62.
Bernsen, H. J., Rijken, P. F., Oostendorp, T., and van der Kogel, A. J. (1995). Vascularity and perfusion of human gliomas xenografted in the athymic nude mouse. Br J Cancer 71, 721-6.
Bowers, D. C., Fan, S., Walter, K. A., Abounader, R., Williams, J. A., Rosen, E. M., and Laterra, J. (2000). Scatter factor/hepatocyte growth factor protects against cytotoxic death in human glioblastoma via phosphatidylinositol 3-kinase- and AKT-dependent pathways. Cancer Res 60, 4277-83.
Chen, H., Centola, M., Altschul, S. F., and Metzger, H. (1998). Characterization of gene expression in resting and activated mast cells. J Exp Med 188, 1657-68.
Guerin, C., Wolff, J. E., Laterra, J., Drewes, L. R., Brem, H., and Goldstein, G. W. (1992). Vascular differentiation and glucose transporter expression in rat gliomas: effects of steroids. Ann Neurol 31, 481-7.
Hobbs, S. K., Monsky, W. L., Yuan, F., Roberts, W. G., Griffith, L., Torchilin, V. P., and Jain, R. K. (1998). Regulation of transport pathways in tumor vessels: role of tumor type and microenvironment. Proc Natl Acad Sci USA 95, 4607-12.
Holash, J., Maisonpierre, P. C., Compton, D., Boland, P., Alexander, C. R., Zagzag, D., Yancopoulos, G. D., and Wiegand, S. J. (1999). Vessel cooption, regression, and growth in tumors mediated by angiopoietins and VEGF. Science 284, 1994-8.
Huminiecki, L., and Bicknell, R. (2000). In silico cloning of novel endothelial-specific genes. Genome Res 10, 1796-806.
Lamszus, K., Laterra, J., Westphal, M., and Rosen, E. M. (1999). Scatter factor/hepatocyte growth factor (SF/HGF) content and function in human gliomas. Int J Dev Neurosci 17, 517-30.
Marx, J. (2001). Caveolae: a once-elusive structure gets some respect. Science 294, 1862-5.
Roberts, W. G., and Palade, G. E. (1997). Neovasculature induced by vascular endothelial growth factor is fenestrated. Cancer Res 57, 765-72.
Shinoura, N., Shamraj, O. I., Hugenholz, H., Zhu, J. G., McBlack, P., Warnick, R., Tew, J. J., Wani, M. A., and Menon, A. G. (1995). Identification and partial sequence of a cDNA that is differentially expressed in human brain tumors. Cancer Lett 89, 215-21.
Smith, R. M., Janet, L. (1988). Lab. Invest. 58, 613-629.
St Croix, B., Rago, C., Velculescu, V., Traverso, G., Romans, K. E., Montgomery, E., Lal, A., Riggins, G. J., Lengauer, C., Vogelstein, B., and Kinzler, K. W. (2000). Genes expressed in human tumor endothelium. Science 289, 1197-202.
Stan, R. V., Arden, K. C., and Palade, G. E. (2001). cDNA and protein sequence, genomic organization, and analysis of cis regulatory elements of mouse and human PLVAP genes. Genomics 72, 304-13.
Tamagnone, L., Artigiani, S., Chen, H., He, Z., Ming, G. I., Song, H., Chedotal, A., Winberg, M. L., Goodman, C. S., Poo, M., Tessier-Lavigne, M., and Comoglio, P. M. (1999). Plexins are a large family of receptors for transmembrane, secreted, and GPI-anchored semaphorins in vertebrates. Cell 99, 71-80.
Vajkoczy, P., and Menger, M. D. (2000). Vascular microenvironment in gliomas. J Neurooncol 50, 99-108.
Vajkoczy, P., Schilling, L., Ullrich, A., Schmiedek, P., and Menger, M. D. (1998). Characterization of angiogenesis and microcirculation of high-grade glioma: an intravital multifluorescence microscopic approach in the athymic nude mouse. J Cereb Blood Flow Metab 18, 510-20.
Vick, N. A., and Bigner, D. D. (1972). Microvascular abnormalities in virally-induced canine brain tumors. Structural bases for altered blood-brain barrier function. J Neurol Sci 17, 29-39.

TABLE 5

| T/N | T/N prob. | SAGE Tag | UG ID | UG description | localization |
|---|---|---|---|---|---|
| 17 | 95 | GTCTCAGTGC | 118893 | Melanoma associated gene MG50 | surface/secreted |
| 14 | 90 | CTTATGCTGC | 82002 | endothelin receptor type B | surface |
| 13 | 99 | CCACCCTCAC | 211573 | HSPG2 Periecan | extracellular |
| 13 | 94 | GTGCTACTTC | 119129 | collagen, type IV, alpha 1 | extracellular |
| 12 | 98 | GAGTGAGACC | 345643 | Thy-1 cell surface antigen | surface |
| 10 | 94 | ATGGCAACAG | 149609 | ITGA5 integrin alpha 5 (Fn receptor) | surface |
| | | | | receptor | |
| 9 | 91 | TCACACAGTG | 23016 | G protein-coupled receptor RDC-1 | surface |
| 8 | 100 | GACCGCAGG | 119129 | collagen, type IV, alpha 1 | extracellular |
| 8 | 97 | GGGAGGGGTG | 2399 | matrix metalloproteinase 14 | extracellular |
| | | | | (membrane-inserted) | |
| 7 | 99 | CCCTACCCTG | 75736 | apolipoprotein D | extracellular |
| 6 | 97 | TTCTCCCAAA | 75617 | collagen, type IV, alpha 2 | extracellular |
| 6 | 98 | GGATGCGCAG | 302741 | Homo sapiens mRNA full length insert cDNA | |
| | | | | clone EU | |
| 5 | 98 | GTGCTAAGCG | 159263 | collagen, type VI, alpha 2 Exon 1 | extracellular |
| 4 | 93 | CCCAGGACAC | 110443 | Homo sapiens cDNA: FLJ22215 fis, clone | |
| | | | | HRC01580. | |

TABLE 6

| | Brain | | | | |
|---|---|---|---|---|---|
| Brain | N/T | | | | |
| N/T | prob | SAGE Tag | UG ID | UG description | Localization |
| 9 | 72 | TAGTTGGAAA | 1119 | nuclear receptor subfamily 4, group A. member 1 | nuclear |
| | | | | NR4A1 | membrane |
| 9 | 72 | AAGGGCGCGG | 1378 | annexin A3 ANXA3 | membrane |
| 9 | 72 | AGCTGTGCCA | 348254 | metallothionein 1A (functional) MT1A | extracellular |
| 7 | 60 | ACAAAATCAA | 110613 | nuclear pore complex interacting protein SMG-1 | membrane |
| 6 | 68 | GCCTGCAGTC | 31439 | serine protease inhibitor, Kunitz type, 2 | extracellular |
| | | | | SPINT2 | |
| 6 | 52 | ACCAGGTCCA | 5167 334549 | solute carrier family 5 (sodium-dependent vitamin | membrane |
| 6 | 52 | GGCTAATTAT | 34114 | ATPase, Na+/K+ transporting, alpha 2 (+) polypepti | membrane |
| 6 | 75 | TTTAAATAGC | 7934 | KLF4 Kruppel-like factor 4 (gut) | intracellular |
| 5 | 81 | CAGTTCATTA | 326035 | early growth response 1 EGR1 | intracellular |
| 5 | 61 | CTGCCGTGAC | 75462 | BTG family, member 2 BTG2 | extracellular |
| 5 | 65 | TTTTAACTTA | 160483 | erythrocyte membrane protein band 7.2 (stomatin) | membrane |
| 4 | 77 | TAGAAACCGG | 8997 | heat shock 70 kD protein 1A HSP70 | intracellular |
| 4 | 77 | CTTCTTGCC | 272572 | hemoglobin, alpha 2 | intracellular |
| | | | 347939 | | |
| 4 | 53 | TAGAAAAAAT | 8906 | syntaxin 7 | surface |

TABLE 7

| Brain | colon | | | | |
|---|---|---|---|---|---|
| T/N | T/N | SAGE Tag | UG ID | UG description | localication |
| 13 | 4 | GTGCTACTTC | 119129 | collagen, type IV, alpha 1 | extracellular |
| 12 | 16 | GAGTGAGACC | 125359 | Thy-1 cell surface antigen | surface |
| 9 | 4 | TCACACAGTG | 23016 | G protein-coupled receptor RDC-1 | surface |
| 8 | 6 | GACCGCAGGA | 119129 | collagen, type IV, alpha 1 | extracellular |
| 8 | 13 | GGGAGGGGTG | 2399 | matrix metalloproteinase 14 (membrane-inserted) | extracellular |
| 7 | 14 | GGGGCTGCCC | 195727 | tumor endothelial marker 1 precursor | surface |
| 6 | 4 | TTCTCCCAAA | 75617 | collagen, type IV, alpha 2 | extracellular |
| 6 | 18 | CCACAGGGGA | 119571 | collagen, type III, alpha 1 (Ehlers-Danlos | extracellular |
| | | | | syndrom | |
| 6 | 9 | TCAAGTTCAC | 351928 | Homo sapiens mRNA full length insert cDNA | |
| | | | | Euroimage 1977059 | |
| 5 | 10 | ACCAAAAACC | 172928 | collagen, type I, alpha 1 | extracellular |
| 4 | 7 | GATCAGGCCA | 119571 | collagen, type III, alpha 1 (Ehlers-Danlos | extracellular |
| | | | | syndrom | |
| 4 | 4 | AGAAACCACG | 119129 | collagen, type IV, alpha 1 | extracellular |

TABLE 8

| Brain | Brain TN | | non-EC | | | |
|---|---|---|---|---|---|---|
| T/N | prob | ShortTag | count | UG ID | UG description | Localization |
| 9 | 83 | AAGGTTCTTC | 1 | 89695 | insulin receptor | surface |
| 7 | 74 | CCCTTTCACA | 1 | 107125 | PV1 | surface |
| 6 | 75 | AGACTAGGGG | 1 | 350065 | Plexin A2 | surface |
| 4 | 69 | CATAAACGGG | 1 | 69954 | laminin, gamma 3 | extracellular |
| 4 | 53 | GGCCAACA17 | 1 | 36353 | Homo sapiens mRNA full length insert | |
| | | | | | cDNA clone EU | |

TABLE 9

| Short Tag | Long Tag | UG ID | UG Description |
|---|---|---|---|
| GTACGTCCCA | GTACGTCCCACCCTGTC | 183556 | solute carrier family 1 (neutral amino acid transp |
| GCAATTTAAC | GCAATTTAACCACATTT | 83974 | solute carrier family 21 (prostaglandin transporte |
| AGGTGCGGGG | AGGTGCGGGGGGCAGAC | 165439 | arsA (bacterial) arsenite transporter, ATP-binding |
| TTTGGGGCTG | TTTGGGGCTGGCCTCAC | 7476 | ATPase, H+ transporting, lysosomal (vacuolar proto |
| CACCCTGTAC | CACCCTGTACAGTTGCC | 25450 | solute carrier family 29 (nucleoside transporters) |
| GGGTGGGCGT | GGGTGGGCGTGCAGGGA | 278378 | karyopherin beta 2b, transportin |

TABLE 10
glioma_tem_only_with_tag

| Unigene ID | Function | LongTag | StdTag | Localization |
|---|---|---|---|---|
| Hs.101382 | tumor necrosis factor, alpha-induced | ACTCAGCCCGGCTGATG | ACTCAGCCCG | cytoplasmic |
| | protein 2 | | | |
| Hs.102135 | signal sequence receptor, delta | GCTCTCTATGCTGACGT | GCTCTCTATG | membrane |
| | (translocon-associated protein delta) | | | |
| Hs.103180 | DC2 protein | AGAATGAAACTGCCGGG | AGAATGAAAC | membrane |
| Hs.105850 | KIAA0404 protein | AAGTGGAATAAACTGCC | AAGTGGAATA | nuclear |
| Hs.10784 | chromosome 6 open reading frame 37 | TTTGAATCAGTGCTAGA | TTTGAATCAG | cytoplasmic |
| Hs.110802 | von Willebrand factor | TTCTGCTCTTGTGCCCT | TTCTGCTCTT | extracellular |
| Hs.112844 | maternally expressed 3 | TGGGAAGTGGGCTCCTT | TGGGAAGTGG | mitochondria |
| Hs.11607 | hypothetical protein FLJ32205 | TGGGCCCGTGTCTGGCC | TGGGCCCGTG | mitochondria |
| Hs.118893 | Melanoma associated gene | ACAACGTCCAGCTGGTG | ACAACGTCCA | extracellular |
| Hs.119120 | E3 ubiquitin ligase SMURF1 | CCCCCTGCCCCTCTGCC | CCCCCTGCCC | mitochondria |
| Hs.121849 | microtubule-associated protein 1 light | GTCTATGCCTCCCAGGA | GTCTATGCCT | nuclear |
| | chain 3 beta | | | |
| Hs.124915 | hypothetical protein MGC2601 | GGCTGGAGCCGCTTTGG | GGCTGGAGCC | extracellular |
| Hs.129780 | tumor necrosis factor receptor | CATACCTCCTGCCCCGC | CATACCTCCT | membrane |
| | superfamily, member 4 | | | |
| Hs.135084 | cystatin C (amyloid angiopathy and | TGCCTGCACCAGGAGAC | TGCCTGCACC | extracellular |
| | cerebral hemorrhage) | | | |
| Hs.136414 | UDP-GlcNAc: betaGal beta-1,3-N- | TTCCTTGTAATCAAAGA | TTCCTTGTAA | extracellular |
| | acetylgluco-saminyl-transferase 5 | | | |
| Hs.137574 | coagulation factor II (thrombin) | TGGCGGCAGAGGCAGAG | TGGCGGCAGA | membrane |
| | receptor-like 3 | | | |
| Hs.148932 | sema domain, transmembrane domain | CCACGTGGCTGGCTGGG | CCACGTGGCT | membrane |
| | (TM), and cytoplasmic domain, | | | |
| | (semaphorin) 6B | | | |
| Hs.149152 | rhophilin 1 | CTGGAGGCTGCCTCGGG | CTGGAGGCTG | nuclear |
| Hs.149609 | integrin, alpha 5 (fibronectin | ATGGCAACAGATCTGGA | ATGGCAACAG | membrane |
| | receptor, alpha polypeptide) | | | |
| Hs.151761 | KIAA0100 gene product | GGTCCCCTACCCTTCCC | GGTCCCCTAC | nuclear |
| Hs.155048 | Lutheran blood group (Auberger b | CCCGCCCCCGCCTTCCC | CCCGCCCCCG | membrane |
| | antigen included) | | | |
| Hs.155223 | stanniocalcin 2 | CCCGAGGCAGAGTCGGG | CCCGAGGCAG | extracellular |
| Hs.155396 | nuclear factor (erythroid-derived 2)- | CTACGTGATGAAGATGG | CTACGTGATG | nuclear |
| | like 2 | | | |
| Hs.155894 | protein tyrosine phosphatase, | ATGGGTTTGCATTTTAG | ATGGGTTTGC | cytoplasmic |
| | non-receptor type 1 | | | |
| Hs.155939 | inositol polyphosphate-5-phosphatase, | ATGGAAGTCTGCGTAAC | ATGGAAGTCT | nuclear |
| | 145 kDa | | | |
| Hs.156351 | hypothetical protein FLJ23471 | TGGACAGCAGGGACCTG | TGGACAGCAG | nuclear |
| Hs.1600 | chaperonin containing TCP1, subunit 5 | TCATAGAAACCTTGATT | TCATAGAAAC | cytoplasmic |
| | (epsilon) | | | |
| Hs.160958 | CDC37 cell division cycle 37 homolog | CAGCGCTGCATTGACTC | CAGCGCTGCA | cytoplasmic |
| | (S. cerevisiae) | | | |
| Hs.165983 | zinc finger protein 335 | CTGGGTGCCCCAGCCTG | CTGGGTGCCC | nuclear |
| Hs.169401 | apolipoprotein E | CGACCCCACGCCACCCC | CGACCCCACG | extracellular |
| Hs.172813 | Rho guanine nucleotide exchange factor | CGCTGGGCGTCTGGGAC | CGCTGGGCGT | nuclear |
| | (GEF) 7 | | | |
| Hs.1735 | inhibin, beta B (activin AB beta | ATTAGTCAGAAACTGCC | ATTAGTCAGA | extracellular |
| | polypeptide) | | | |
| Hs.180324 | insulin-like growth factor binding | GATAGCACAGTTGTCAG | GATAGCACAG | extracellular |
| | protein 5 | | | |
| Hs.180610 | splicing factor proline/glutamine rich | CGTACTGAGCGCTTTGG | CGTACTGAGC | nuclear |
| | (polypyrimidine tract binding protein | | | |
| | associated) | | | |
| Hs.18069 | legumain | GGGGCTTCTGTAGCCCC | GGGGCTTCTG | extracellular |
| Hs.180842 | ribosomal protein L13 | CCCGTCCGGAACGTCTA | CCCGTCCGGA | nuclear |
| Hs.180920 | ribosomal protein S9 | CCAGTGGCCCGGAGCTG | CCAGTGGCCC | mitochondria |
| Hs.182248 | sequestosome 1 | ACTGTACTCCAGCCTAG | ACTGTACTCC | cytoplasmic |
| Hs.1827 | nerve growth factor receptor (TNFR | AGCTCCAGACCCCCAGC | AGCTCCAGAC | membrane |
| | superfamily, member 16) | | | |
| Hs.184245 | SMART/HDAC1 associated repressor protein | GACTCGCAGACACCGGG | GACTCGCAGA | nuclear |
| Hs.184669 | zinc finger protein 144 (Mel-18) | GGCCTCCAGCCACCCAC | GGCCTCCAGC | nuclear |
| Hs.19347 | mitochondrial ribosomal protein L45 | GACCAGCCTTCAGATGG | GACCAGCCTT | cytoplasmic |
| Hs.194654 | brain-specific angiogenesis inhibitor 1 | GCCCCCAGGGGCAGGAC | GCCCCCAGGG | membrane |
| Hs.19555 | prostate tumor over expressed gene 1 | GAGGATGGTGTCCTGAG | GAGGATGGTG | cytoplasmic |
| Hs.195851 | actin, alpha 2, smooth muscle, aorta | AAGATCAAGATCATTGC | AAGATCAAGA | cytoplasmic |
| Hs 201671 | SRY (sex determining region Y)-box 13 | AGCACAGGGTCGGGGGG | AGCACAGGGT | membrane |
| Hs.20225 | tuftelin interacting protein 11 | GCCAAGTGAACTGTGGC | GCCAAGTGAA | cytoplasmic |
| Hs.202833 | heme oxygenase (decycling) 1 | CGTGGGTGGGGAGGGAG | CGTGGGTGGG | membrane |
| Hs.20976 | Homo sapiens cDNA FLJ34888 fis, clone | CTCCCCTATGGACTGGC | CTCCCCTATG | |
| | NT2NE2017332 | | | |
| Hs 211600 | tumor necrosis factor, alpha-induced | AGTATGAGGAAATCTCT | AGTATGAGGA | nuclear |
| | protein 3 | | | |
| Hs.212680 | tumor necrosis factor receptor | GCCCCCTTCCTCCCTTG | GCCCCCTTCC | membrane |
| | superfamily, member 18 | | | |
| Hs.21595 | DNA segment on chromosome X and Y | GGGATTTCTGTGTCTGC | GGGATTTCTG | nuclear |
| | (unique) 155 expressed sequence | | | |
| Hs.217493 | annexin A2 | CTTCCAGCTAACAGGTC | CTTCCAGCTA | nuclear |
| Hs.2250 | leukemia inhibitory factor (cholinergic | GCCTTGGGTGACAAATT | GCCTTGGGTG | extracellular |
| | differentiation factor) | | | |
| Hs.23131 | kinesin family member C3 | GCCTCCCGCCACGGGGC | GCCTCCCGCC | nuclear |
| Hs.2340 | junction plakoglobin | GTGTGGGGGGCTGGGGG | GTGTGGGGGG | nuclear |
| Hs.234726 | serine (or cysteine) proteinase | GACTCTTCAGTCTGGAG | GACTCTTCAG | extracellular |
| | inhibitor, Glade A (alpha-1 anti- | | | |
| | proteinase, antitrypsin), member 3 | | | |
| Hs.236516 | C-type (calcium dependent, carbohydrate- | GCCACACCCACCGCCCC | GCCACACCCA | membrane |
| | recognition domain) lectin, superfamily | | | |
| | member 9 | | | |
| Hs.240443 | multiple endocrine neoplasia I | CCAGGGCAACAGAATGA | CCAGGGCAAC | nuclear |
| Hs.25450 | solute carrier family 29 (nucleoside | CACCCTGTACAGTTGCC | CACCCTGTAC | membrane |
| | transporters), member 1 | | | |
| Hs.25590 | stanniocalcin 1 | GACGAATATGAATGTCA | GACGAATATG | extracellular |
| Hs.25590 | stanniocalcin 1 | CAAACTGGTCTAGGTCA | CAAACTGGTC | extracellular |
| Hs.25590 | stanniocalcin 1 | GTAATGACAGATGCAAG | GTAATGACAG | extracellular |
| Hs.268571 | apolipoprotein C-I | TGGCCCCAGGTGCCACC | TGGCCCCAGG | extracellular |
| Hs.272927 | Sec23 homolog A (S. cerevisiae) | AACACAATCATATGATG | AACACAATCA | cytoplasmic |
| Hs.274184 | transcription factor binding to IGHM | GAGGGTATACTGAGGGG | GAGGGTATAC | nuclear |
| | enhancer 3 | | | |
| Hs.274453 | likely ortholog of mouse embryonic | GGAGCCAGCTGACCTGC | GGAGCCAGCT | membrane |
| | epithelial gene 1 | | | |
| Hs.27836 | likely ortholog of mouse fibronectin | GAGCCTCAGGTGCTCCC | GAGCCTCAGG | membrane |
| | type III repeat containing protein 1 | | | |
| Hs.278573 | CD59 antigen p18-20 (antigen identified | TACTTCACATACAGTGC | TACTTCACAT | extracellular |
| | by monoclonal antibodies 16.3A5, | | | |
| | EJ16, EJ30, EL32 and G344) | | | |
| Hs.286035 | myosin XVB, pseudogene | CGGTGGGACCACCCTGC | CGGTGGGACC | nuclear |
| Hs.286035 | myosin XVB, pseudogene | GGAGAAACAGCTGCTGA | GGAGAAACAG | nuclear |
| Hs.288203 | Homo sapiens, clone IMAGE: 4845226, mRNA | GCTCAGGTCTGCCGGGG | GCTCAGGTCT | |
| Hs.288991 | TNFAIP3 interacting protein 2 | TCTGCACTGAGAAACTG | TCTGCACTGA | nuclear |
| Hs,296406 | KIAA0685 gene product | TCCACGCCCTTCCTGGC | TCCACGCCCT | nuclear |
| Hs.29716 | hypothetical protein FLJ10980 | TTGCAATAGCAAAACCC | TTGCAATAGC | nuclear |
| Hs.297753 | vimentin | TCCAAATCGATGTGGAT | TCCAAATCGA | mitochondria |
| Hs.29797 | ribosomal protein L10 | AGGGCTTCCAATGTGCT | AGGGCTTCCA | mitochondria |
| Hs.299257 | ESTs, Weakly similar to hypothetical | AACCTGGGAGGTGGAGG | AACCTGGGAG | |
| | protein FLJ20489 [Homo sapiens] | | | |
| | [H. sapiens] | | | |
| Hs.301242 | likely ortholog of mouse myocytic | GGCCAACATTTGGTCCA | GGCCAACATT | cytoplasmic |
| | induction/differentiation originator | | | |
| Hs.301685 | KIAA0620 protein | GGGGCTGGAGGGGGGCA | GGGGCTGGAG | membrane |
| Hs.302741 | Homo sapiens mRNA full length insert | GGATGCGCAGGGGAGGC | GGATGCGCAG | |
| | cDNA clone EUROIMAGE 50374 | | | |
| Hs.318751 | ESTs, Weakly similar to T21371 | GAAGACACTTGGTTTGA | GAAGACACTT | |
| | hypothetical protein F25H8.3- | | | |
| | Caenorhabditis elegans [C. elegans] | | | |
| Hs.321231 | UDP-Gal: betaGlcNAc beta 1,4-galactosyl- | GAGAGAAGAGTGATCTG | GAGAGAAGAG | extracellular |
| | transferase, polypeptide 3 | | | |
| Hs.326445 | v-akt murine thymoma viral oncogene | GCAGGGTGGGGAGGGGT | GCAGGGTGGG | cytoplasmic |
| | homolog 2 | | | |
| Hs.334604 | KIAA1870 protein | TCAGTGTATTAAAACCC | TCAGTGTATT | extracellular |
| Hs.339283 | endoplasmic reticulum associated protein | ATACTATAATTGTGAGA | ATACTATAAT | nuclear |
| | 140 kDa | | | |
| Hs.34516 | ceramide kinase | GCTGGTTCCTGAGTGGC | GCTGGTTCCT | cytoplasmic |
| Hs.348000 | ESTs, Weakly similar to hypothetical | AGCCACTGCGCCCGGCC | AGCCACTGCG | |
| | protein FLJ20489 [Homo sapiens] | | | |
| | [H. sapiens] | | | |
| Hs.350065 | hypothetical protein FLJ30634 | AGACTAGGGGCCGGAGC | AGACTAGGGG | nuclear |
| Hs.352535 | KIAA0943 protein | GGGACAGCTGTCTGTGG | GGGACAGCTG | cytoplasmic |
| Hs.352949 | ESTs, Weakly similar to hypothetical | AACCCAGGAGGCGGAGC | AACCCAGGAG | |
| | protein FLJ20489 [Homo sapiens] | | | |
| | [H. sapiens] | | | |
| Hs.353002 | ESTs | CAGCCTGAGGCTCTTGG | CAGCCTGAGG | |
| Hs.353193 | LOC124402 | CCTCCCCTGCACCTGGG | CCTCCCCTGC | nuclear |
| Hs.363027 | Homo sapiens cDNA FLJ39848 fis, clone | GCTTCAGTGGGGGAGAG | GCTTCAGTGG | |
| | SPLEN2014669 | | | |
| Hs.367653 | hypothetical protein FLJ22329 | TGTTTGGGGGCTTTTAG | TGTTTGGGGG | extracellular |
| Hs.373548 | Homo sapiens cDNA: FLJ22720 fis, | TTTTAAATTAGGTTTTG | TTTTAAATTA | |
| | clone HSI14320 | | | |
| Hs.374415 | ESTs | ATCTCAAAGATACACAG | ATCTCAAAGA | |
| Hs.39619 | hypothetical protein L0057333 | TTTGTGGGCAGTCAGGC | TTTGTGGGCA | extracellular |
| Hs.39871 | myosin ID | ATTGTAGACAATGAGGG | ATTGTAGACA | nuclear |
| Hs.400429 | ESTs | GCAAAACCCTGCTCTCC | GCAAAACCCT | |
| Hs.401975 | ESTs, Weakly similar to T17346 | GTCTCAGTGCTGAGGCG | GTCTCAGTGC | |
| | hypothetical protein DKFZp58601624.1- | | | |
| | human (fragment) [H. sapiens] | | | |
| Hs.405289 | ESTs, Weakly similar to hypothetical | AGCCACTGTGCCCGGCC | AGCCACTGTG | |
| | protein F1120378 [Homo sapiens] | | | |
| | [H. sapiens] | | | |
| Hs.406068 | ubiquitin-conjugating enzyme E2M | TGATTAAGGTCGGCGCT | TGATTAAGGT | nuclear |
| | (UBC12 homolog, yeast) | | | |
| Hs.406507 | sprouty homolog 4 (Drosophila) | TTACAAACAGAAAAGCT | TTACAAACAG | extracellular |
| Hs.41716 | endothelial cell-specific molecule 1 | TTTATTATTGTTCAATA | TTTATTATTG | extracellular |
| Hs.45008 | hypothetical protein DKFZp547N157 | CGGGCCTCAGGTGGCAG | CGGGCCTCAG | nuclear |
| Hs.4980 | LIM domain binding 2 | TAAAGGCACAGTGGCTC | TAAAGGCACA | nuclear |
| Hs.5307 | synaptopodin | ATATTAGGAAGTCGGGG | ATATTAGGAA | nuclear |
| Hs.56205 | insulin induced gene 1 | TGATTAAAACAAGTTGC | TGATTAAAAC | membrane |
| Hs.57958 | EGF-TM7-latrophilin-related protein | TTGTGCACGCATCAGTG | TTGTGCACGC | membrane |
| Hs.61490 | schwannomin interacting protein 1 | CCTGCCTCGTAGTGAAG | CCTGCCTCGT | nuclear |
| Hs.61638 | myosin X | CAAAACTGTTTGTTGGC | CAAAACTGTT | nuclear |
| Hs.62192 | coagulation factor III (thromboplastin, | TAGGAAAGTAAAATGGA | TAGGAAAGTA | membrane |
| | tissue factor) | | | |
| Hs.65238 | ring finger protein 40 | CTCCATCGGCTGTGAGG | CTCCATCGGC | nuclear |
| Hs.6657 | Hermansky-Pudlak syndrome 4 | CAAGCATCCCCGTTCCA | CAAGCATCCC | nuclear |
| Hs.6831 | golgi complex associated protein 1, | GAGTTAGGCACTTCCTG | GAGTTAGGCA | nuclear |
| | 60 kDa | | | |
| Hs.69954 | laminin, gamma 3 | CATAAACGGGCACACCC | CATAAACGGG | extracellular |
| Hs.7187 | hypothetical protein FLJ10707 | TTGCCTGGGATGCTGGT | TTGCCTGGGA | nuclear |
| Hs.73798 | macrophage migration inhibitory factor | AACGCGGCCAATGTGGG | AACGCGGCCA | cytoplasmic |
| | (glycosylation-inhibiting factor) | | | |
| Hs.73818 | ubiquinol-cytochrome c reductase hinge | GGTTTGGCTTAGGCTGG | GGTTTGGCTT | nuclear |
| | protein | | | |
| Hs.74471 | gap junction protein, alpha 1, 43 kDa | GATTTTTGTGGTGTGGG | GATTTTTGTG | membrane |
| | (connexin 43) | | | |
| Hs.74566 | dihydropyrimidinase-like 3 | GGCTGCCCTGGGCAGCC | GGCTGCCCTG | cytoplasmic |
| Hs.74602 | aquaporin 1 (channel-forming integral | ATGGCAACAGAAACCAA | ATGGCAACAG | membrane |
| | protein, 28 kDa) | | | |
| Hs.75093 | procollagen-lysine, 2-oxoglutarate | AGAGCAAACCGTAGTCC | AGAGCAAACC | extracellular |
| | 5-dioxy-genase (lysine hydroxylase, | | | |
| | Ehlers-Danlos syndrome type VI) | | | |
| Hs.75445 | SPARC-like 1 (mast9, hevin) | TGCACTTCAAGAAAATG | TGCACTTCAA | extracellular |
| Hs.75736 | apolipoprotein D | CCCTACCCTGTTACCTT | CCCTACCCTG | extracellular |
| Hs.76353 | serine (or cysteine) proteinase | GGAAAAATGTTGGAATG | GGAAAAATGT | extracellular |
| | inhibitor, Glade A (alpha-1 anti- | | | |
| | proteinase, antitrypsin), member 5 | | | |
| Hs.7718 | hypothetical protein FLJ22678 | GTTTTTGCTTCAGCGGC | GTTTTTGCTT | extracellular |
| Hs.77313 | cyclin-dependent kinase (CDC2-like) 10 | GAGGACCCAACAGGAGG | GAGGACCCAA | cytoplasmic |
| Hs.77326 | insulin-like growth factor binding | ACTGAGGAAAGGAGCTC | ACTGAGGAAA | extracellular |
| | protein 3 | | | |
| Hs.77573 | uridine phosphorylase | TGCAGCGCCTGCGGCCT | TGCAGCGCCT | nuclear |
| Hs.77864 | KIAA0638 protein | CTGGGGGGAAGGGACTG | CTGGGGGGAA | nuclear |
| Hs.77886 | lamin A/C | GTGCCTGAGAGGCAGGC | GTGCCTGAGA | nuclear |
| Hs.77886 | lamin A/C | TCACAGGGTCCCCGGGG | TCACAGGGTC | nuclear |
| Hs.77886 | lamin A/C | GGAGGGGGCTTGAAGCC | GGAGGGGGCT | nuclear |
| Hs.78056 | cathespin L | GGAGGAATTCATCTTCA | GGAGGAATTC | extracellular |
| Hs.78531 | similar to RIKEN cDNA 5730528L 13 gene | GAAAGTGGCTGTCCTGG | GAAAGTGGCT | nuclear |
| Hs.78575 | prosaposin (variant Gaucher disease and | TCCCTGGCTGTTGAGGC | TCCCTGGCTG | extracellular |
| | variant metachromatic leukodystrophy) | | | |
| Hs.82575 | small nuclear ribonucleoprotein | AAGATGAGGGGGCAGGC | AAGATGAGGG | nuclear |
| | polypeptide B″ | | | |
| Hs.82749 | transmembrane 4 superfamily member 2 | CCAACAAGAATGCATTG | CCAACAAGAA | membrane |
| Hs.83126 | TAF11 RNA polymerase II, TATA box | AAGGATGCGGTGATGGC | AAGGATGCGG | nuclear |
| | binding protein (TBP)-associated factor, | | | |
| | 28 kDa | | | |
| Hs.83169 | matrix metalloproteinase 1 (interstitial | TGCAGTCACTGGTGTCA | TGCAGTCACT | extracellular |
| | collagenase) | | | |
| Hs.83384 | S100 calcium binding protein, beta | GCCGTGTAGACCCTAAC | GCCGTGTAGA | cytoplasmic |
| | (neural) | | | |
| Hs.83484 | SRY (sex determining region Y)-box 4 | CAGGCTTTTTGGCTTCC | CAGGCTTTTT | nuclear |
| Hs.83484 | SRY (sex determining region Y)-box 4 | TCCCTGGGCAGCTTCAG | TCCCTGGGCA | nuclear |
| Hs.83727 | cleavage and polyadenylation specific | GAGCGCAGCGAGCTAGC | GAGCGCAGCG | nuclear |
| | factor 1, 160 kDa | | | |
| Hs.84063 | Homo sapiens cDNA: FLJ23507 fis, | CAGGTGGTTCTGCCATC | CAGGTGGTTC | |
| | clone LNG03128 | | | |
| Hs.84753 | hypothetical protein FLJ12442 | GCCCACATCCGCTGAGG | GCCCACATCC | cytoplasmic |
| Hs.89695 | insulin receptor | AAGGTTCTTCTCAAGGG | AAGGTTCTTC | membrane |

TABLE 11
Glioma Repressed in Transporters

| Short Tag | Long Tag | UG ID | UG Description |
|---|---|---|---|
| GGCTAATTAT** | GGCTAATTATCATCAAT | 34114 | ATPase, Na+/K+ transporting alpha 2(+) polypeptide |
| CAAAAATAAA | CAAAAATAAAAGCCGA | 30246 | solute carrier family 19 (thlamine transporter), m |
| | | | *Transport* |

**Also present in Glioma repressed list

TABLE 12
Nuclear Brain and Colon Proteins

| Unigene ID | Function | OMIMID | Protein |
|---|---|---|---|
| Hs.149098 | smoothelin | 602127 | NP_599031 |
| Hs.197298 | NS1-binding protein | | AAG43485 |
| Hs.337986 | hypothetical protein MGC4677 | | NP_443103 |

TABLE 13
Cytoplasmic Brain/Colon Proteins

| Unigene ID | Function | OMIMID | Protein |
|---|---|---|---|
| Hs.327412 | TEM 15, COL3A1, Homo | | |
| | sapiens clone FLC1492 | | |
| | PRO3121 mRNA, complete cds | | |
| Hs.75721 | profilin 1 | 176610 | NP_005013 |

TABLE 14
Extracellular Colon/Brain Proteins

| Unigene ID | Function | OMIMID | Protein |
|---|---|---|---|
| Hs.1103 | transforming growth factor, beta | 190180 | NP_000651 |
| | 1 (Camurati-Engelmann | | |
| | disease) | | |
| Hs.111779 | secreted protein, acidic, | 182120 | NP_003109 |
| | cysteine-rich (osteonectin) | | |
| Hs.119129 | collagen, type IV, alpha 1 | 120130 | NP_001836 |
| Hs.119571 | collagen, type III, alpha 1 | 120180 | NP_000081 |
| | (Ehlers-Danlos syndrome type | | |
| | IV, autosomal dominant | | |
| Hs.151738 | matrix metalloproteinase 9 | 120361 | NP_004985 |
| | (gelatinase B, 92 kDa | | |
| | gelatinase, 92 kDa type IV | | |
| | collagenase) | | |
| Hs.159263 | collagen, type VI, alpha 2 | 120240 | NP_001840 |
| Hs.172928 | collagen, type I, alpha 1 | 120150 | NP_000079 |
| Hs.179573 | TEM 40, COL1A2 alt polyA; | 120160 | NP_000080 |
| | involved in tissue remodeling | | |
| Hs.75617 | collagen, type IV, alpha 2 | 120090 | NP_001837 |
| Hs.78672 | laminin, alpha 4 | 600133 | NP_002281 |
| Hs.821 | biglycan | 301870 | NP_001702 |

TABLE 15
Membrane Brain/Colon Proteins

| Unigene ID | Function | OMIMID | Protein |
|---|---|---|---|
| Hs.125359 | TEM 13, Thy-1 cell surface | 188230 | NP_006279 |
| | antigen | | |
| Hs.185973 | degenerative spermatocyte | | NP_003667 |
| | homolog, lipid desaturase | | |
| | (Drosophila) | | |
| Hs.195727 | TEM 1, endosialin | 606064 | NP_065137 |
| Hs.23016 | G protein-coupled receptor | | |
| Hs.2399 | matrix metalloproteinase 14 | 600754 | NP_004986 |
| | (membrane-inserted) | | |
| Hs.285814 | sprouty homolog 4 (Drosophilia) | | AAK00653 |
| Hs.82002 | endothelin receptor type B | 131244 | NP_000106 |

TABLE 16
Brain and Colon Proteins

| Unigene ID | Function | OMIMID | Protein |
|---|---|---|---|
| Hs.1103 | transforming growth factor, beta 1 (Camurati- | 190180 | NP_000651 |
| | Engelmann disease) | | |
| Hs.111779 | secreted protein, acidic, cysteine-rich | 182120 | NP_003109 |
| | (osteonectin) | | |
| Hs.119129 | collagen, type IV, alpha 1 | 120130 | NP_001836 |
| Hs.119571 | collagen, type III, alpha 1 (Ehlers-Danlos | 120180 | NP_000081 |
| | syndrome type IV, autosomal dominant) | | |
| Hs.125359 | TEM 13, Thy-1 cell surface antigfen | 188230 | NP_006279 |
| Hs.149098 | smoothelin | 602127 | NP_599031 |
| Hs.151738 | matrix metalloproteinase 9 (gelatinase B, | 120361 | NP_004985 |
| | 92 kDa gelatinase, 92 kDa type IV collagenase) | | |
| Hs.159263 | collagen, type VI, alpha 2 | 120240 | NP_001840 |
| Hs.172928 | collagen, type 1, alpha 1 | 120150 | NP_000079 |
| Hs.179573 | TEM 40, COL1A2 alt polyA; involved in tissue | 120160 | NP_000080 |
| | remodeling | | |
| Hs.185973 | degenerative spermatocyte homolog, lipid | | NP_003667 |
| | desaturase (Drosophila) | | |
| Hs.195727 | TEM 1, endosialin | 606064 | NP_065137 |
| Hs.197298 | NS1-binding protein | | AAG43485 |
| Hs.23016 | G protein-coupled receptor | | |
| Hs.2399 | matrix metalloproteinase 14 (membrane- | 600754 | NP_004986 |
| | inserted) | | |
| Hs.285814 | sprouty homolog 4 (Drosophila) | | AAK00653 |
| Hs.327412 | TEM15, COL311, Homo sapiens clone | | |
| | FLC1492 PRO3121 mRNA, complete cds | | |
| Hs.337986 | hypotehtical protein MGC4677 | | NP_443103 |
| Hs.351928 | Homo sapiens mRNA full length insert cDNA | | |
| | clone EUROIMAGE 1977059 | | |
| Hs.356096 | ESTs, Highly similar to hypothetical protein | | |
| | FLJ10350 [Homo sapiens] [H. sapiens] | | |
| Hs.75617 | collagen, type IV, alpha 2 | 120090 | NP_001837 |
| Hs.75721 | profilin 1 | 176610 | NP_005013 |
| Hs.78672 | laminin, alpha 4 | 600133 | NP_002281 |
| Hs.82002 | endothelin receptor type B | 131244 | NP_000106 |
| Hs.821 | biglycan | 301870 | NP_001702 |

TABLE 17
Additional Tumor Endothelial Markers in Brain

| Unigene ID | Function | |
|---|---|---|
| Hs.326445 | v-akt murine thymoma vial | Protein Kinase |
| | oncogene homolog 2 | |
| Hs.77313 | cyclin-dependent kinase | Protein Kinase |
| | (cdc2-like) 10 | |
| Hs.301242 | ortholog mouse myocytic | Non-Protein Kinase |
| | induction/differntiation originator | |
| Hs.194654 | brain-specific angiogenesis | Membrane GPCR |
| | inhibitor 1 | |
| Hs.57958 | EGF-RM7 latrophilin-related | Membrane GPCR |
| | protein | |
| Hs.148932 | sema domain | Receptors with Short |
| | | Cytoplasmic Tail |
| Hs.149609 | integrin, alpha 5 | Receptors with Short |
| | | Cytoplasmic Tail |
| Hs.27836 | likely ortholog of mouse | Receptors with Short |
| | fibronectin type III | Cytoplasmic Tail |
| Hs.155048 | Lutheran blood group (Auberger | Receptors with Short |
| | b antigen included) | Cytoplasmic Tail |
| Hs.102135 | SSR4, TRAPD | Receptors with Short |
| | | Cytoplasmic Tail |
| Hs.1827 | nerve growth factor receptor | Membrane Receptor |
| | (TNFR superfamily, member 16) | |
| Hs.41716 | insulin-like growth factor | Extracellular Growth |
| | binding protein | Factors & Cytokine |
| Hs.2250 | leukemia inhibitor factor | Extracellular Growth |
| | | Factors & Cytokine |
| Hs.155894 | protein typrosine phosphatase, | Cell-Selective |
| | nonreceptor type I | Phosphatase |

TABLE 18
Cytoplasmic GEMs

| Unigene ID | Function | OMIMID | Protein |
|---|---|---|---|
| Hs.111611 | ribosomal protein L27 | 607526 | NP_000979 |
| Hs.160958 | CDC37 cell division cycle 37 | 605065 | NP_008996 |
| | homolog (S. cerevisiae) | | |
| Hs.327412 | TEM15, COLI3A1, Homo | | |
| | sapiens clone FLC1492 | | |
| | PRO3121 mRNA, complete cds | | |
| Hs.34516 | ceramide kinase | | NP_073603 |
| Hs.352535 | KIAA0943 protein | | BAA76787 |
| Hs.61661 | F-box only protein 32 | 606604 | NP_478136 |
| Hs.73798 | macrophage migration | 153620 | NP_002406 |
| | inhibitory factor (glycosylation- | | |
| | inhibiting factor) | | |
| Hs.75721 | profilin 1 | 176610 | NP_005013 |
| Hs.83384 | S100 calcium binding protein, | 176990 | NP_006263 |
| | beta (neural) | | |

TABLE 19
Nuclear GEMs

| Unigene | | | |
|---|---|---|---|
| ID | Function | OMIMID | Protein |
| Hs.105850 | KIAA0404 protein | | BAA23700 |
| Hs.110443 | hypothetical protein FLJ22215 | | NP_073745 |
| Hs.121849 | microtubule-associated protein | | NP_073729 |
| | 1 light chain 3 beta | | |
| Hs.129673 | eukaryotic translation initiation | 602641 | NP_001407 |
| | factor 4A, isoform 1 | | |
| Hs.149098 | smoothelin | 602127 | NP_599031 |
| Hs.155396 | nuclear factor (erythroid-derived | 600492 | NP_006155 |
| | 2)-like 2 | | |
| Hs.172813 | Rho guanine nucleotide exchange | 605477 | NP_663788 |
| | factor (GEF) 7 | | |
| Hs.197298 | NS1-binding protein | | AAG43485 |
| Hs.211600 | tumor necrosis factor, alpha- | 191163 | NP_006281 |
| | induced protein 3 | | |
| Hs.217493 | annexin A2 | 151740 | — |
| Hs.2340 | junction plakoglobin | 173325 | NP_002221 |
| Hs.274184 | transcription factor binding | 314310 | NP_006512 |
| | to IGHM enhancer 3 | | |
| Hs.286035 | myosin XVB, pseudogene | | |
| Hs.332173 | transducin-like enhancer of split 2 | 601041 | NP_003251 |
| | (E(sp1) homolog, Drosophila) | | |
| Hs.337986 | hypothetical protein MGC4677 | | NP_443103 |
| Hs.339283 | endoplasmic reticulum associated | | |
| | protein 140 kDa | | |
| Hs.350065 | hypothetical protein FLJ30634 | | NP_694559 |
| Hs.65238 | ring finger protein 40 | | NP_055586 |
| Hs.6657 | Hermansky-Pudlak syndrome 4 | 606682 | BAB33337 |
| Hs.75061 | MARCKS-like protein | 602940 | NP_075385 |
| Hs.77573 | uridine phosphorylase | 191730 | NP_003355 |
| Hs.77886 | lamin A/C | 150330 | NP_005563 |

TABLE 20
Membrane GEMs

| Unigene ID | Function | OMIMID | Protein |
|---|---|---|---|
| Hs.107125 | plasmalemina vesicle | | NP_112600 |
| | associated protein | | |
| Hs.125359 | TEM13, Thy-1 cell | 188230 | NP_006279 |
| | surface antigen | | |
| Hs.137574 | coagulation factor II | 602779 | NP_003941 |
| | (thrombin) receptor-like 3 | | |
| Hs.143897 | dysferlin, limb girdle | 603009 | NP_003485 |
| | muscular dystrophy 2B | | |
| | (autosomal recessive) | | |
| Hs.148932 | sema domain, | | NP_115484 |
| | transmembrane domain | | |
| | (TM), and cytoplasmic | | |
| | domain, (semaphorin) 6B | | |
| Hs.149609 | integrin, alpha 5 | 135620 | NP_002196 |
| | (fibronectin receptor, | | |
| | alpha polypeptide) | | |
| Hs.166254 | likely ortholog of rat | | NP_112200 |
| | vacuole membrane | | |
| | protein 1 | | |
| Hs.1827 | nerve growth factor | 162010 | NP_002498 |
| | receptor (TNFR | | |
| | superfamily, member 16) | | |
| Hs.185973 | degenerative | | NP_003667 |
| | spermatocyte homolog, | | |
| | lipid desaturase | | |
| | (Drosophila) | | |
| Hs.195727 | TEM1, endosialin | 606064 | NP_065137 |
| Hs.202833 | heme oxygenase | 141250 | NP_002124 |
| | (decycling) 1 | | |
| Hs.23016 | G protein-coupled | | |
| | receptor | | |
| Hs.236516 | C-type (calcium | | NP_055173 |
| | dependent, carbohydrate- | | |
| | recognition domain) | | |
| | lectin, superfamily | | |
| | member 9 | | |
| Hs.2399 | matrix metalloproteinase | 600754 | NP_004986 |
| | 14 (membrane-inserted) | | |
| Hs.25450 | solute carrier family 29 | 602193 | NP_004946 |
| | (nucleoside transporters), | | |
| | member 1 | | |
| Hs.274453 | likely ortholog of mouse | | NP_060081 |
| | embryonic epithelial gene 1 | | |
| Hs.277477 | major histocompatibility | 142840 | NP_002108 |
| | complex, class I, C | | |
| Hs.27836 | likely ortholog of mouse | | NP_073734 |
| | fibronectin type III repeat | | |
| | containing protein 1 | | |
| Hs.285814 | sprouty homolog 4 | | AAK00653 |
| | (Drosophila) | | |
| Hs.301685 | KIAA0620 protein | | BAA31595 |
| Hs.62192 | coagulation factor III | 134390 | NP_001984 |
| | (thromboplastin, tissue | | |
| | factor) | | |
| Hs.74602 | aquaporin 1 (channel- | 110450 | AAH22486 |
| | forming integral protein, | | |
| | 28 kDa) | | |
| Hs.77961 | major histocompatibility | 142830 | NP_005505 |
| | complex, class I, B | | |
| Hs.79356 | Lysosomal-associated | 601476 | NP_006753 |
| | multispanning membrane | | |
| | protein-5 | | |
| Hs.82002 | endothelin receptor type B | 131244 | NP_000106 |
| Hs.89695 | insulin receptor | 147670 | NP_000199 |
| Hs.97199 | complement component | 120577 | NP_036204 |
| | 1, q subcomponent, | | |
| | receptor 1 | | |

TABLE 21
Extracellular GEMS

| Unigene ID | Function | OMIMID | Protein |
|---|---|---|---|
| Hs.1103 | transforming growth factor, | 190180 | NP_000651 |
| | beta 1 (Camurati-Engelmann | | |
| | disease) | | |
| Hs.110802 | von Willebrand factor | 193400 | NP_000543 |
| Hs.111779 | secreted protein, acidic, | 182120 | NP_003109 |
| | cysteine-rich (osteonectin) | | |
| Hs.119129 | collagen, type IV, alpha 1 | 120130 | NP_001836 |
| Hs.119571 | collagen, type III, alpha 1 | 120180 | NP_000081 |
| | (Ehlers-Danlos syndrome type | | |
| | IV, autosomal dominant) | | |
| Hs.135084 | cystatin C (amyloid angiopathy | 604312 | NP_000090 |
| | and cerebral hemorrhage) | | |
| Hs.136414 | UDP-GlcNAc:betaGal beta-1,3- | | NP_114436 |
| | N- | | |
| | acetylglucosaminyltransferase 5 | | |
| Hs.151738 | matrix metalloproteinase 9 | 120361 | NP_004985 |
| | (gelatinase B, 92 kDa | | |
| | gelatinase, 92 kDa type IV | | |
| | collagenase) | | |
| Hs.159263 | collagen, type VI, alpha 2 | 120240 | NP_001840 |
| Hs.169401 | apolipoprotein E | 107741 | NP_000032 |
| Hs.172928 | collagen, type I, alpha 1 | 120150 | NP_000079 |
| Hs.1735 | inhibin, beta B (activin AB beta | 147390 | NP_002184 |
| | polypeptide) | | |
| Hs.179573 | TEM40, COL1A2 all polyA; | 120160 | NP_000080 |
| | involved in tissue remodeling | | |
| Hs.180324 | insulin-like growth factor | 146734 | |
| | binding protein 5 | | |
| Hs.18069 | legumain | 602620 | NP_005597 |
| Hs.211573 | heparan sulfate proteoglycan 2 | 142461 | NP_005520 |
| | (perlecan) | | |
| Hs.25590 | stanniocalcin 1 | 601185 | NP_003146 |
| Hs.268571 | apolipoprotein C-I | 107710 | |
| Hs.321231 | UDP-Gal:betaGlcNAc beta 1,4- | 604014 | NP_003770 |
| | galactosyltransferase, | | |
| | polypeptide 3 | | |
| Hs.365706 | matrix Gla protein | 154870 | NP_000891 |
| Hs.367653 | hypothetical protein FLJ22329 | | |
| Hs.69954 | laminin, gamma 3 | 604349 | NP_006050 |
| Hs.73817 | chemokine (C-C motif) ligand 3 | 182283 | NP_002974 |
| Hs.75111 | protease, serine, 11 (IGF | 602194 | NP_002766 |
| | binding) | | |
| Hs.75445 | SPARC-like 1 (mast9, hevin) | 606041 | NP_004675 |
| Hs.75617 | collagen, type IV, alpha 2 | 120090 | NP_001837 |
| Hs.75736 | apolipoprotein D | 107740 | NP_001638 |
| Hs.7718 | hypothetical protein FLJ22678 | | NP_078812 |
| Hs.77326 | insulin-like growth factor | 146732 | NP_000589 |
| | binding protein 3 | | |
| Hs.78575 | prosaposin (variant Gaucher | 176801 | NP_002769 |
| | disease and variant | | |
| | metachromatic | | |
| | leukodystrophy) | | |
| Hs.78672 | laminin, alpha 4 | 600133 | NP_002281 |
| Hs.82085 | serine (or cysteine) proteinase | 173360 | NP_000593 |
| | inhibitor, clade E (nexin, | | |
| | plasminogen activator inhibitor | | |
| | type 1), member 1 | | |
| Hs.821 | biglycan | 301870 | NP_001702 |
| Hs.83169 | matrix metalloproteinase 1 | 120353 | NP_002412 |
| | (interstitial collagenase) | | |
| Hs.90107 | adhesion regulating molecule 1 | | NP_008933 |

TABLE 22
Brain tumor markers unsorted

| Unigene ID | Function | OMIMID | Protein |
|---|---|---|---|
| Hs.105850 | KIAA0404 protein | | BAA23700 |
| Hs.107125 | plasmalemma vesicle | | NP_112600 |
| | associated protein | | |
| Hs.1103 | transforming growth factor, | 190180 | NP_000651 |
| | beta 1 (Camurati-Engelmann | | |
| | disease) | | |
| Hs.110443 | hypothetical protein FLJ22215 | | NP_073745 |
| Hs.110802 | von Willebrand factor | 193400 | NP_000543 |
| Hs.111611 | ribosomal protein L27 | 607526 | NP_000979 |
| Hs.111779 | secreted protein, acidic, | 182120 | NP_003109 |
| | cysteine-rich (osteonectin) | | |
| Hs.11607 | hypothetical protein FLJ32205 | | NP_689774 |
| Hs.119129 | collagen, type IV, alpha 1 | 120130 | NP_001836 |
| Hs.119571 | collagen, type III, alpha 1 | 120180 | NP_000081 |
| | (Ehlers-Danlos syndrome type | | |
| | IV, autosomal dominant) | | |
| Hs.121849 | microtubule-associated | | NP_073729 |
| | protein 1 light chain 3 beta | | |
| Hs.125359 | TEM13, Thy-1 cell surface | 188230 | NP_006279 |
| | antigen | | |
| Hs.127824 | ESTs, Weakly similar to | | |
| | CA28_HUMAN Collagen | | |
| | alpha 2(VIII) chain | | |
| | (Endothelial collagen) | | |
| | [H. sapiens] | | |
| Hs.129673 | eukaryotic translation initiation | 602641 | NP_001407 |
| | factor 4A, isoform 1 | | |
| Hs.135084 | cystatin C (amyloid | 604312 | NP_000090 |
| | angiopathy and cerebral | | |
| | hemorrhage) | | |
| Hs.136414 | UDP-GlcNAc:betaGal beta- | | NP_114436 |
| | 1,3-N- | | |
| | acetylglucosaminyltransferase 5 | | |
| Hs.137574 | coagulation factor II | 602779 | NP_003941 |
| | (thrombin) receptor-like 3 | | |
| Hs.143897 | dysferlin, limb girdle muscular | 603009 | NP_003485 |
| | dystrophy 2B (autosomal | | |
| | recessive) | | |
| Hs.148932 | sema domain, | | NP_115484 |
| | transmembrane domain (TM), | | |
| | and cytoplasmic domain, | | |
| | (semaphorin) 6B | | |
| Hs.149098 | smoothelin | 602127 | NP_599031 |
| Hs.149609 | integrin, alpha 5 (fibronectin | 135620 | NP_002196 |
| | receptor, alpha polypeptide) | | |
| Hs.151738 | matrix metalloproteinase 9 | 120361 | NP_004985 |
| | (gelatinase B, 92 kDa | | |
| | gelatinase, 92 kDa type IV | | |
| | collagenase) | | |
| Hs.155396 | nuclear factor (erythroid- | 600492 | NP_006155 |
| | derived 2)-like 2 | | |
| Hs.159263 | collagen, type VI, alpha 2 | 120240 | NP_001840 |
| Hs.160958 | CDC37 cell division cycle 37 | 605065 | NP_008996 |
| | homolog (S. cerevisiae) | | |
| Hs.166254 | likely ortholog of rat vacuole | | NP_112200 |
| | membrane protein 1 | | |
| Hs.169401 | apolipoprotein E | 107741 | NP_000032 |
| Hs.172813 | Rho guanine nucleotide | 605477 | NP_663788 |
| | exchange factor (GEF) 7 | | |
| Hs.172928 | collagen, type I, alpha 1 | 120150 | NP_000079 |
| Hs.1735 | inhibin, beta B (activin AB | 147390 | NP_002184 |
| | beta polypeptide) | | |
| Hs.179573 | TEM40, COL1A2 alt polyA; | 120160 | NP_000080 |
| | involved in tissue remodeling | | |
| Hs.180324 | insulin-like growth factor | 146734 | |
| | binding protein 5 | | |
| Hs.18069 | legumain | 602620 | NP_005597 |
| Hs.180920 | ribosomal protein S9 | 603631 | |
| Hs.1827 | nerve growth factor receptor | 162010 | NP_002498 |
| | (TNFR superfamily, member | | |
| | 16) | | |
| Hs.185973 | degenerative spermatocyte | | NP_003667 |
| | homolog, lipid desaturase | | |
| | (Drosophila) | | |
| Hs.195727 | TEM1, endosialin | 606064 | NP_065137 |
| Hs.197298 | NS1-binding protein | | AAG43485 |
| Hs.202833 | heme oxygenase (decycling) 1 | 141250 | NP_002124 |
| Hs.20976 | Homo sapiens cDNA | | |
| | FLJ34888 fis, clone | | |
| | NT2NE2017332 | | |
| Hs.211573 | heparan sulfate proteoglycan | 142461 | NP_005520 |
| | 2 (perlecan) | | |
| Hs.211600 | tumor necrosis factor, alpha- | 191163 | NP_006281 |
| | induced protein 3 | | |
| Hs.217493 | annexin A2 | 151740 | |
| Hs.23016 | G protein-coupled receptor | | |
| Hs.2340 | junction plakoglobin | 173325 | NP_002221 |
| Hs.236516 | C-type (calcium dependent, | | NP_055173 |
| | carbohydrate-recognition | | |
| | domain) lectin, superfamily | | |
| | member 9 | | |
| Hs.2399 | matrix metalloproteinase 14 | 600754 | NP_004986 |
| | (membrane-inserted) | | |
| Hs.25450 | solute carrier family 29 | 602193 | NP_004946 |
| | (nucleoside transporters), | | |
| | member 1 | | |
| Hs.25590 | stanniocalcin 1 | 601185 | NP_003146 |
| Hs.268571 | apolipoprotein C-I | 107710 | |
| Hs.274184 | transcription factor binding to | 314310 | NP_006512 |
| | IGHM enhancer 3 | | |
| Hs.274453 | likely ortholog of mouse | | NP_060081 |
| | embryonic epithelial gene 1 | | |
| Hs.277477 | major histocompatibility | 142840 | NP_002108 |
| | complex, class I, C | | |
| Hs.27836 | likely ortholog of mouse | | NP_073734 |
| | fibronectin type III repeat | | |
| | containing protein 1 | | |
| Hs.285814 | sprouty homolog 4 | | AAK00653 |
| | (Drosophila) | | |
| Hs.286035 | myosin XVB, pseudogene | | |
| Hs.288203 | Homo sapiens, clone | | |
| | IMAGE: 4845226, mRNA | | |
| Hs.29797 | ribosomal protein L10 | 312173 | NP_115617 |
| Hs.299257 | ESTs, Weakly similar to | | |
| | hypothetical protein FLJ20489 | | |
| | [Homo sapiens] [H. sapiens] | | |
| Hs.301685 | KIAA0620 protein | | BAA31595 |
| Hs.302741 | Homo sapiens mRNA full | | |
| | length insert cDNA clone | | |
| | EUROIMAGE 50374 | | |
| Hs.318751 | ESTs, Weakly similar to | | |
| | T21371 hypothetical protein | | |
| | F25H8.3 - Caenorhabditis | | |
| | elegans [C. elegans] | | |
| Hs.321231 | UDP-Gal:betaGlcNAc beta | 604014 | NP_003770 |
| | 1,4-galactosyltransferase, | | |
| | polypeptide 3 | | |
| Hs.327412 | TEM15, COL3A1, Homo | | |
| | sapiens clone FLC1492 | | |
| | PRO3121 mRNA, complete | | |
| | cds | | |
| Hs.332173 | transducin-like enhancer of | 601041 | NP_003251 |
| | split 2 (E(sp1) homolog, | | |
| | Drosophila) | | |
| Hs.337986 | hypothetical protein MGC4677 | | NP_443103 |
| Hs.339283 | endoplasmic reticulum | | |
| | associated protein 140 kDa | | |
| Hs.34516 | ceramide kinase | | NP_073603 |
| Hs.350065 | hypothetical protein FLJ30634 | | NP_694559 |
| Hs.351928 | Homo sapiens mRNA full | | |
| | length insert cDNA clone | | |
| | EUROIMAGE 1977059 | | |
| Hs.352535 | KIAA0943 protein | | BAA76787 |
| Hs.352949 | ESTs, Weakly similar to | | |
| | hypothetical protein FLJ20489 | | |
| | [Homo sapiens] [H. sapiens] | | |
| Hs.356096 | ESTs, Highly similar to | | |
| | hypothetical protein FLJ10350 | | |
| | [Homo sapiens] [H. sapiens] | | |
| Hs.363027 | Homo sapiens cDNA | | |
| | FLJ39848 fis, clone | | |
| | SPLEN2014669 | | |
| Hs.365706 | matrix Gla protein | 154870 | NP_000891 |
| Hs.367653 | hypothetical protein FLJ22329 | | |
| Hs.374415 | ESTs | | |
| Hs.380983 | ESTs, Highly similar to | | |
| | ITB1_HUMAN Integrin beta-1 | | |
| | precursor (Fibronectin | | |
| | receptor beta subunit) (CD29) | | |
| | (Integrin VLA-4 beta subunit) | | |
| | [H. sapiens] | | |
| Hs.400429 | ESTs | | |
| Hs.401975 | ESTs, Weakly similar to | | |
| | T17346 hypothetical protein | | |
| | DKFZp586O1624.1 - human | | |
| | (fragment) [H. sapiens] | | |
| Hs.61661 | F-box only protein 32 | 606604 | NP_478136 |
| Hs.62192 | coagulation factor III | 134390 | NP_001984 |
| | (thromboplastin, tissue factor) | | |
| Hs.65238 | ring finger protein 40 | | NP_055586 |
| Hs.6657 | Hermansky-Pudlak syndrome 4 | 606682 | BAB33337 |
| Hs.69954 | laminin, gamma 3 | 604349 | NP_006050 |
| Hs.73798 | macrophage migration | 153620 | NP_002406 |
| | inhibitory factor (glycosylation- | | |
| | inhibiting factor) | | |
| Hs.73817 | chemokine (C-C motif) ligand 3 | 182283 | NP_002974 |
| Hs.74602 | aquaporin 1 (channel-forming | 110450 | AAH22486 |
| | integral protein, 28 kDa) | | |
| Hs.75061 | MARCKS-like protein | 602940 | NP_075385 |
| Hs.75111 | protease, serine, 11 (IGF | 602194 | NP_002766 |
| | binding) | | |
| Hs.75445 | SPARC-like 1 (mast9, hevin) | 606041 | NP_004675 |
| Hs.75617 | collagen, type IV, alpha 2 | 120090 | NP_001837 |
| Hs.75721 | prolilin 1 | 176610 | NP_005013 |
| Hs.75736 | apolipoprotein D | 107740 | NP_001638 |
| Hs.7718 | hypothetical protein FLJ22678 | | NP_078812 |
| Hs.77326 | insulin-like growth factor | 146732 | NP_000589 |
| | binding protein 3 | | |
| Hs.77573 | uridine phosphorylase | 191730 | NP_003355 |
| Hs.77886 | lamin A/C | 150330 | NP_005563 |
| Hs.77961 | major histocompatibility | 142830 | NP_005505 |
| | complex, class I, B | | |
| Hs.78575 | prosaposin (variant Gaucher | 176801 | NP_002769 |
| | disease and variant | | |
| | metachromatic | | |
| | leukodystrophy) | | |
| Hs.78672 | laminin, alpha 4 | 600133 | NP_002281 |
| Hs.79356 | Lysosomal-associated | 601476 | NP_006753 |
| | multispanning membrane | | |
| | protein-5 | | |
| Hs.82002 | endothelin receptor type B | 131244 | NP_000106 |
| Hs.82085 | serine (or cysteine) proteinase | 173360 | NP_000593 |
| | inhibitor, clade E (nexin, | | |
| | plasminogen activator | | |
| | inhibitor type 1), member 1 | | |
| Hs.821 | biglycan | 301870 | NP_001702 |
| Hs.83169 | matrix metalloproteinase 1 | 120353 | NP_002412 |
| | (interstitial collagenase) | | |
| Hs.83384 | S100 calcium binding protein, | 176990 | NP_006263 |
| | beta (neural) | | |
| Hs.84063 | Homo sapiens cDNA: | | |
| | FLJ23507 fis, clone | | |
| | LNG03128 | | |
| Hs.89695 | insulin receptor | 147670 | NP_000199 |
| Hs.90107 | adhesion regulating molecule 1 | | NP_008933 |
| Hs.97199 | complement component 1, q | 120577 | NP_036204 |
| | subcomponent, receptor 1 | | |

We claim:

1. A method to aid in diagnosing glioma, comprising the steps of:
detecting an expression product of at least one gene in a first brain tissue sample suspected of being neoplastic wherein said at least one gene is selected from the group consisting of signal sequence receptor, delta (translocon-associated protein delta); DC2 protein; KIAA0404 protein; symplekin; Huntingtin interacting protein I; plasmalemma vesicle associated protein; KIAA0726 gene product; latexin protein; transforming growth factor, beta 1; hypothetical protein FLJ22215; Rag C protein; hypothetical protein FLJ23471; N-myristoyltransferase 1; hypothetical protein dJ1181N3.1; ribosomal protein L27; Hs 111988; Hs 112238; laminin, alpha 5; protective protein for beta-galactosidase (galactosialidosis); Melanoma associated gene; Melanoma associated gene; E3 ubiquitin ligase SMURF1; collagen, type IV, alpha 1; collagen, type IV, alpha 1; collagen, type IV, alpha 1; insulin-like growth factor binding protein 7; gene predicted from cDNA with a complete coding sequence; Thy-1 cell surface antigen; Hs 127824; GTP binding protein 2; Homo sapiens mRNA; cDNA DKFZp586D0918 (from clone DKFZp586D0918); cutaneous T-cell lymphoma-associated tumor antigen se20-4; differentially expressed nucleolar TGF-beta1 target protein (DENTT); dysferlin, limb girdle muscular dystrophy 2B (autosomal recessive); smoothelin; integrin, alpha 5 (fibronectin receptor, alpha polypeptide); putative translation initiation factor; retinoic acid induced 14; matrix metalloproteinase 9 (gelatinase B, 92 kD gelatinase, 92 kD type IV collagenase); Lutheran blood group (Auberger b antigen included); stanniocalcin 2; nuclear factor (erythroid-derived 2)-like 2; protein tyrosine phosphatase, non-receptor type 1; integrin, alpha 10; collagen, type VI, alpha 2; chromosome 21 open reading frame 25; CDC37 (cell division cycle 37, S. cerevisiae, homolog); Hs 16450; Rho guanine nucleotide exchange factor (GEF) 7; creatine kinase, brain; hypothetical protein FLJ10297; hypothetical protein FLJ10350; TNF-induced protein; tumor necrosis factor receptor superfamily, member 12 (translocating chain-association membrane protein); cofilin 1 (non-muscle); splicing factor proline/glutamine rich (polypyrimidine tract-binding protein-associated); splicing factor proline/glutamine rich (polypyrimidine tract-binding protein-associated); v-ets avian erythroblastosis virus E26 oncogene homolog 1; protease, cysteine, 1 (legumain); ribosomal protein L13; chromosome 22 open reading frame 5; zinc finger protein 144 (MeI-18); degenerative spermatocyte (homolog Drosophila; lipid desaturase); eukaryotic translation initiation factor 2C, 2; mitochondrial ribosomal protein L45; prostate tumor over expressed gene 1; NADH dehydrogenase (ubiquinone) 1 alpha subcomplex, 7 (14.5 kD, B14.5a); glioma endothelial marker 1 precursor; NS1-binding protein; ribosomal protein L38; tuftelin-interacting protein; HLA class II region expressed gene KE2; translocase of inner mitochondrial membrane 17 homolog A (yeast); sudD (suppressor of bimD6, Aspergillus nidulans) homolog; heparan sulfate proteoglycan 2 (perlecan); SEC24 (S. cerevisiae) related gene family, member A; NADH dehydrogenase (ubiquinone) Fe—S protein 7 (20 kD) (NADH-coenzyme Q reductase); DNA segment on chromosome X and Y (unique) 155 expressed sequence; annexin A2; Homo sapiens clone 24670 mRNA sequence; matrix metalloproteinase 10 (stromelysin 2); KIAA1049 protein; G protein-coupled receptor; hypothetical protein FLJ20401; matrix metalloproteinase 14 (membrane-inserted); KIAA0470 gene product; solute carrier family 29 (nucleoside transporters), member 1; stanniocalcin 1; stanniocalcin 1; stanniocalcin 1; tumor suppressor deleted in oral cancer-related 1; tumor suppressor deleted in oral cancer-related 1; apolipoprotein C—I; glutathione peroxidase 4 (phospholipid hydroperoxidase); Hs 272106; transcription factor binding to IGHM enhancer 3; hypothetical protein DKFZp762A227; hypothetical protein FLJ22362; CD59 antigen p18-20 (antigen identified by monoclonal antibodies 16.3A5, EJ16, EJ30, EL32 and G344); PRO0628 protein; melanoma-associated antigen recognised by cytotoxic T lymphocytes; LOC88745; Homo sapiens beta-1,3-galactosyltransferase-6 (B3GALT6) mRNA, complete cds; sprouty (Drosophila) homolog 4; sprouty (Drosophila) homolog 4; Homo sapiens mRNA; cDNA DKFZp434E1515 (from clone DKFZp434E1515); coactosin-like protein; hypothetical protein FLJ21865; Hs296234; KIAA0685 gene product; hypothetical protein FLJ10980; ribosomal protein L10; ribosomal protein S19; Hs 299251; Huntingtin interacting protein K; Homo sapiens mRNA full length insert cDNA clone EUROIMAGE 50374; Hs 311780; Hs 212191; v-akt murine thymoma viral oncogene homolog 2; Hs 328774; transducin-like enhancer of split 2, homolog of Drosophila E(sp1); KIAA1870 protein; ribosomal protein L10a; peptidylprolyl isomerase A (cyclophilin A); Hs 344224; hypothetical protein FLJ23239; hypothetical protein DKFZp761H221; KIAA1887 protein; Homo sapiens mRNA full length insert cDNA clone EUROIMAGE 701679; Homo sapiens cDNA FLJ30634 fis, clone CTONG2002453; Homo sapiens cDNA FLJ32203 fis, clone PLACE6003038, weakly similar to ZINC FINGER PROTEIN 84; Homo sapiens mRNA full length insert cDNA clone EUROIMAGE 1035904; hypothetical protein L0057333; myosin ID; plexin B2; lectin, galactoside-binding, soluble, 8 (galectin 8); double ring-finger protein, Dorfin; DKFZP434B168 protein; LIM domain binding 2; integrin beta 4 binding protein; synaptopodin; Hs 54828; insulin induced gene 1; acetyl LDL receptor; SREC; excision repair cross-complementing rodent repair deficiency, complementation group 1 (includes overlapping antisense sequence); hypothetical protein FLJ22329; schwannomin-interacting protein 1; PTEN induced putative kinase 1; myosin X; Homo sapiens cDNA FLJ32424 fis, clone SKMUS2000954, moderately similar to Homo sapiens F-box protein Fbx25 (FBX25) 97; golgi phosphoprotein 1; splicing factor, arginine/serine-rich 6; laminin, gamma 3; cysteine-rich protein 2; U6 snRNA-associated Sm-like protein LSm7; hypothetical protein FLJ10707; Homo sapiens, Similar to RIKEN cDNA 2310012N15 gene, clone IMAGE:3342825, mRNA, partial cds; macrophage migration inhibitory factor (glycosylation-inhibiting factor); ubiquinol-cytochrome c reductase hinge protein; gap junction protein, alpha 1, 43 kD (connexin 43); dihydropyrimidinase-like 3; aquaporin 1 (channel-forming integral protein, 28 kD); protein expressed in thyroid; macrophage myristoylated alanine-rich C kinase substrate; procollagen-lysine, 2-oxoglutarate 5-dioxygenase (lysine hydroxylase, Ehlers-Danlos syndrome type VI); protease, serine, 11 (IGF binding); 24-dehydrocholesterol reductase; collagen, type IV, alpha 2; profilin 1; apolipoprotein D; hyaluronoglucosaminidase 2; hypothetical protein FLJ22678; quiescin Q6; ras homolog gene family, member A; ras homolog gene family, member A; plasminogen activator, urokinase; insulin-like growth factor binding protein 3; uridine phosphorylase; KIAA0638 protein; B7 homolog 3; lamin A/C; lamin A/C; lamin A/C; regulator of G-protein signalling 12; proteasome (prosome, macropain) 26S subunit, non-ATPase, 8; Homo sapiens, Similar to RIKEN cDNA 5730528L13 gene, clone MGC:17337 IMAGE:4213591, mRNA, complete cds; prosaposin (variant Gaucher disease and variant metachromatic leukodystrophy); laminin, alpha 4; transcription elongation factor A (SII), 1; lectin, galactoside-binding, soluble, 3 binding protein; ribosomal protein S16; glycophorin C (Gerbich blood group); endothelin receptor type B; serine (or cysteine) proteinase inhibitor, Glade E (nexin, plasminogen activator inhibitor type 1), member 1; biglycan; small nuclear ribonucleoprotein polypeptide B″; transmembrane 4 superfamily member 2; TAF11 RNA polymerase II, TATA box binding protein (TBP)-associated factor, 28 kD; lysyl oxidase-like 2; SRY (sex determining region Y)-box 4; SOX4 SRY (sex determining region Y)-box 4; SRY (sex determining region Y)-box 4; actin related protein 2/3 complex, subunit 2 (34 kD); Homo sapiens cDNA: FLJ23507 fis, clone LNG03128; hypothetical protein FLJ12442; Fas (TNFRSF6)— associated via death domain; mitogen-activated protein kinase kinase kinase 11; TEK tyrosine kinase, endothelial (venous malformations, multiple cutaneous and mucosal); insulin receptor; cell membrane glycoprotein, 110000M(r) (surface antigen); Homo sapiens cDNA FLJ11863 fis, clone HEMBA1006926; jagged 1 (Alagille syndrome); KIAA0304 gene product; pre-B-cell leukemia transcription factor 2; Homo sapiens cDNA FLJ31238 fis, clone KIDNE2004864; p53-induced protein; complement component 1, q subcomponent, receptor 1; complement component 1, q subcomponent, receptor 1; apolipoprotein E; chemokine (C—C motif) ligand 3; coagulation factor II (thrombin) receptor-like 3; coagulation factor III (thromboplastin, tissue factor); collagen, type I, alpha 1; collagen, type III, alpha 1 (Ehlers-Danlos syndrome type IV, autosomal dominant); C-type (calcium dependent, carbohydrate-recognition domain) lectin, superfamily member 9; cystatin C (amyloid angiopathy and cerebral hemorrhage); endoplasmic reticulum associated protein 140 kDa; ESTs; ESTs; ESTs, Highly similar to hypothetical protein FLJ10350 [Homo sapiens] [H. sapiens]; ESTs, Highly similar to ITB1_HUMAN Integrin beta-1 precursor (Fibronectin receptor beta subunit) (CD29) (Integrin VLA-4 beta subunit) [H. sapiens]; ESTs, Weakly similar to hypothetical protein FLJ20489 [Homo sapiens] [H. sapiens]; ESTs, Weakly similar to T17346 hypothetical protein DKFZp586O1624.1—human (fragment) [H. sapiens]; ESTs, Weakly similar to T21371 hypothetical protein F25H8.3—Caenorhabditis elegans [C. elegans]; eukaryotic translation initiation factor 4A, isoform 1; heme oxygenase (decycling) 1; Hermansky-Pudlak syndrome 4; Homo sapiens cDNA FLJ34888 fis, clone NT2NE2017332; Homo sapiens cDNA FLJ39848 fis, clone SPLEN2014669; Homo sapiens mRNA full length insert cDNA clone EUROIMAGE 1977059; Homo sapiens, clone IMAGE:4845226, mRNA; hypothetical protein FLJ22329; hypothetical protein FLJ32205; hypothetical protein MGC4677; inhibin, beta B (activin AB beta polypeptide); insulin-like growth factor binding protein 5; junction plakoglobin; KIAA0620 protein; KIAA0943 protein; likely ortholog of rat vacuole membrane protein 1; Lysosomal-associated multispanning membrane protein-5; major histocompatibility complex, class I, B; major histocompatibility complex, class I, C; matrix Gla protein; matrix metalloproteinase 1 (interstitial collagenase); microtubule-associated protein 1 light chain 3 beta; nerve growth factor receptor (TNFR superfamily, member 16); ribosomal protein S9; ring finger protein 40; S100 calcium binding protein, beta (neural); sema domain, transmembrane domain (TM), and cytoplasmic domain, (semaphorin) 6B; SPARC-like 1 (mast9, hevin); tumor necrosis factor, alpha-induced protein 3; UDP-Gal:betaGlcNAc beta 1,4-galactosyltransferase, polypeptide 3; UDP-GlcNAc:betaGal beta-1,3-N-acetylglucosaminyltransferase 5; von Willebrand factor; v-akt murine thymoma vial oncogene homolog 2; cyclin-dependent kinase (cdc2-like) 10; ortholog mouse myocytic induction/differentiation originator; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16); insulin-like growth factor binding protein; leukemia inhibitory factor; protein tyrosine phosphatase, nonreceptor type I; and Homo sapiens, clone IMAGE:3908182, mRNA, partial cds; and
comparing expression of the at least one gene in the first brain tissue sample with expression of the at least one gene in a second brain tissue sample which is normal, wherein increased expression of the at least one gene in the first brain tissue sample relative to the second tissue sample identifies the first brain tissue sample as likely to be neoplastic.

2. The method of claim 1 wherein the increased expression of the at least one gene in the first brain tissue sample relative to the second tissue sample is at least two-fold higher.

3. The method of claim 1 wherein the increased expression of the at least one gene in the first brain tissue sample relative to the second tissue sample is at least five-fold higher.

4. The method of claim 1 wherein the increased expression of the at least one gene in the first brain tissue sample relative to the second tissue sample is at least ten-fold higher.

5. The method of claim 1 wherein the expression product is RNA.

6. The method of claim 1 wherein the expression product is protein.

7. The method of claim 1 wherein the first and second tissue samples are from a human.

8. The method of claim 1 wherein the first and second tissue samples are from the same human.

9. The method of claim 6 wherein the step of detecting is performed using a Western blot.

10. The method of claim 6 wherein the step of detecting is performed using an immunoassay.

11. The method of claim 6 wherein the step of detecting is performed using an immunohistochemical assay.

12. The method of claim 5 wherein the step of detecting is performed using SAGE.

13. The method of claim 5 wherein the step of detecting is performed using hybridization to a microarray.

14. A method of treating a glioma, comprising the step of:
contacting cells of the glioma with an antibody, wherein the antibody specifically binds to an extracellular epitope of a protein selected from the group consisting of plasmalemma vesicle associated protein; KIAA0726 gene product; laminin, alpha 5; collagen, type IV, alpha 1; insulin-like growth factor binding protein 7; Thy-1 cell surface antigen; dysferlin, limb girdle muscular dystrophy 2B; integrin, alpha 5; matrix metalloproteinase 9; Lutjheran blood group, integrink, alpha 10, collagen, type VI, alpha 2; glioma endothelial marker 1 precursor; translocase of inner mitochondrial membrane 17 homolog A; heparan sulfate proteoglycan 2; annexin A2; matrix metalloproteinase 10; G protein-coupled receptor; matrix metalloproteinase 14; solute carrier family 29, member 1; CD59 antigen p18-20; KIAA 1870 protein; plexin B2; lectin, glactoside-binding, soluble, 8; integrin beta 4 binding protein; acetyl LDL receptor; laminin, gamma 3; macrophage migration inhibitory factor; gap junction p roein, alpha 1, 43 kD; aquaporin 1; protease, serine, 11; collagen, type IV, alpha 2; apolipoprotein D; plasminogen activator, urokinase; insulin-like growth factor binding protein 3; regulator of G-protein signaling 12; prosaposin; laminin, alpha 4; lectin, galactoside-binding, soluble, 3 binding protein; glycophorin C; endothelin receptor type B; biglycan; transmembrane 4 superfamilyh member 2; lysyl osidase-like 2; TEK tyrosine kinase, endothelial; insulin receptore; cell membrane glycoprotein, 110000M(r); jagged 1; plasmalemma vesicle associated protein; TEM13, Thy-1 cell surface antigen; coagulation factor II (thrombin) receptor-like 3; dysferlin, limb girdle muscular dystrophy 2B (autosomal recessive); sema domain, transmembrane domain (TM), and cytoplasmic domain, (semaphorin) 6B; integrin, alpha 5 (fibronectin receptor, alpha polypeptide); likely ortholog of rat vacuole membrane protein 1; nerve growth factor receptor (TNFR superfamily, member 16); degenerative spermatocyte homolog, lipid desaturase (Drosophila); TEM1, endosialin; heme oxygenase (decycling) 1; G protein-coupled receptor; C-type (calcium dependent, carbohydrate-recognition domain) lectin, superfamily member 9; matrix metalloproteinase 14 (membrane-inserted); solute carrier family 29 (nucleoside transporters), member 1; likely ortholog of mouse embryonic epithelial gene 1; major histocompatibility complex, class I, C; likely ortholog of mouse fibronectin type III repeat containing protein 1; sprouty homolog 4 (Drosophila); KIAA0620 protein; coagulation factor III (thromboplastin, tissue factor); aquaporin 1 (channel-forming integral protein, 28 kDa); major histocompatibility complex, class I, B; Lysosomal-associated multispanning membrane protein-5; endothelin receptor type B; insulin receptor; complement component 1, q subcomponent, receptor 1; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16) and complement component 1, q subcomponent, receptor 1; whereby immune destruction of cells of the glioma is triggered.

15. The method of claim 14 wherein the antibody is conjugated to a diagnostic or therapeutic reagent.

16. The method of claim 14 wherein the glioma is multidrug-sensitive.

17. The method of claim 15 wherein the reagent is a chemotherapeutic agent.

18. The method of claim 15 wherein the reagent is a cytotoxin.

19. The method of claim 15 wherein the reagent is a non-radioactive label.

20. The method of claim 15 wherein the reagent is a radioactive compound.

21. The method of claim 14 wherein the glioma is in a human.

22. A method of identifying a test compound as a potential anti-cancer or anti-glioma drug, comprising the step of:
contacting a test compound with a cell which expresses at least one gene selected from the group consisting of signal sequence receptor, delta (translocon-associated protein delta); DC2 protein; KIAA0404 protein; symplekin; Huntingtin interacting protein I; plasmalemma vesicle associated protein; KIAA0726 gene product; latexin protein; transforming growth factor, beta 1; hypothetical protein FLJ22215; Rag C protein; hypothetical protein FLJ23471; N-myristoyltransferase 1; hypothetical protein dJ1181N3.1; ribosomal protein L27; Hs 111988; Hs 112238; laminin, alpha 5; protective protein for beta-galactosidase (galactosialidosis); Melanoma associated gene; Melanoma associated gene; E3 ubiquitin ligase SMURF1; collagen, type IV, alpha 1; collagen, type IV, alpha 1; collagen, type IV, alpha 1; insulin-like growth factor binding protein 7; gene predicted from cDNA with a complete coding sequence; Thy-1 cell surface antigen; Hs 127824; GTP binding protein 2; Homo sapiens mRNA; cDNA DKFZp586D0918 (from clone DKFZp586D0918); cutaneous T-cell lymphoma-associated tumor antigen se20-4; differentially expressed nucleolar TGF-beta1 target protein (DENTT); dysferlin, limb girdle muscular dystrophy 2B (autosomal recessive); smoothelin; integrin, alpha 5 (fibronectin receptor, alpha polypeptide); putative translation initiation factor; retinoic acid induced 14; matrix metalloproteinase 9 (gelatinase B, 92 kD gelatinase, 92 kD type IV collagenase); Lutheran blood group (Auberger b antigen included); stanniocalcin 2; nuclear factor (erythroid-derived 2)-like 2; protein tyrosine phosphatase, non-receptor type 1; integrin, alpha 10; collagen, type VI, alpha 2; chromosome 21 open reading frame 25; CDC37 (cell division cycle 37, S. cerevisiae, homolog); Hs 16450; Rho guanine nucleotide exchange factor (GEF) 7; creatine kinase, brain; hypothetical protein FLJ10297; hypothetical protein FLJ10350; TNF-induced protein; tumor necrosis factor receptor superfamily, member 12 (translocating chain-association membrane protein); cofilin 1 (non-muscle); splicing factor proline/glutamine rich (polypyrimidine tract-binding protein-associated); splicing factor proline/glutamine rich (polypyrimidine tract-binding protein-associated); v-ets avian erythroblastosis virus E26 oncogene homolog 1; protease, cysteine, 1 (legumain); ribosomal protein L13; chromosome 22 open reading frame 5; zinc finger protein 144 (MeI-18); degenerative spermatocyte (homolog Drosophila; lipid desaturase); eukaryotic translation initiation factor 2C, 2; mitochondrial ribosomal protein L45; prostate tumor over expressed gene 1; NADH dehydrogenase (ubiquinone) 1 alpha subcomplex, 7 (14.5 kD, B14.5a); glioma endothelial marker 1 precursor; NS1-binding protein; ribosomal protein L38; tuftelin-interacting protein; HLA class II region expressed gene KE2; translocase of inner mitochondrial membrane 17 homolog A (yeast); sudD (suppressor of bimD6, Aspergillus nidulans) homolog; heparan sulfate proteoglycan 2 (perlecan); SEC24 (S. cerevisiae) related gene family, member A; NADH dehydrogenase (ubiquinone) Fe—S protein 7 (20 kD) (NADH-coenzyme Q reductase); DNA segment on chromosome X and Y (unique) 155 expressed sequence; annexin A2; Homo sapiens clone 24670 mRNA sequence; matrix metalloproteinase 10 (stromelysin 2); KIAA1049 protein; G protein-coupled receptor; hypothetical protein FLJ20401; matrix metalloproteinase 14 (membrane-inserted); KIAA0470 gene product; solute carrier family 29 (nucleoside transporters), member 1; stanniocalcin 1; stanniocalcin 1; stanniocalcin 1; tumor suppressor deleted in oral cancer-related 1; tumor suppressor deleted in oral cancer-related 1; apolipoprotein C—I; glutathione peroxidase 4 (phospholipid hydroperoxidase); Hs 272106; transcription factor binding to IGHM enhancer 3; hypothetical protein DKFZp762A227; hypothetical protein FLJ22362; CD59 antigen p18-20 (antigen identified by monoclonal antibodies 16.3A5, EJ16, EJ30, EL32 and G344); PRO0628 protein; melanoma-associated antigen recognised by cytotoxic T lymphocytes; LOC88745; Homo sapiens beta-1,3-galactosyltransferase-6 (B3GALT6) mRNA, complete cds; sprouty (Drosophila) homolog 4; sprouty (Drosophila) homolog 4; Homo sapiens mRNA; cDNA DKFZp434E1515 (from clone DKFZp434E1515); coactosin-like protein; hypothetical protein FLJ21865; Hs296234; KIAA0685 gene product; hypothetical protein FLJ10980; ribosomal protein L10; ribosomal protein S19; Hs 299251; Huntingtin interacting protein K; Homo sapiens mRNA full length insert cDNA clone EUROIMAGE 50374; Hs 311780; Hs 212191; v-akt murine thymoma viral oncogene homolog 2; Hs 328774; transducin-like enhancer of split 2, homolog of Drosophila E(sp1); KIAA1870 protein; ribosomal protein L10a; peptidylprolyl isomerase A (cyclophilin A); Hs 344224; hypothetical protein FLJ23239; hypothetical protein DKFZp761H221; KIAA1887 protein; Homo sapiens mRNA full length insert cDNA clone EUROIMAGE 701679; Homo sapiens cDNA FLJ30634 fis, clone CTONG2002453; Homo sapiens cDNA FLJ32203 fis, clone PLACE6003038, weakly similar to ZINC FINGER PROTEIN 84; Homo sapiens mRNA full length insert cDNA clone EUROIMAGE 1035904; hypothetical protein L0057333; myosin ID; plexin B2; lectin, galactoside-binding, soluble, 8 (galectin 8); double ring-finger protein, Dorfin; DKFZP434B 168 protein; LIM domain binding 2; integrin beta 4 binding protein; synaptopodin; Hs 54828; insulin induced gene 1; acetyl LDL receptor; SREC; excision repair cross-complementing rodent repair deficiency, complementation group 1 (includes overlapping antisense sequence); hypothetical protein FLJ22329; schwannomin-interacting protein 1; PTEN induced putative kinase 1; myosin X; Homo sapiens cDNA FLJ32424 fis, clone SKMUS2000954, moderately similar to Homo sapiens F-box protein Fbx25 (FBX25) 97; golgi phosphoprotein 1; splicing factor, arginine/serine-rich 6; laminin, gamma 3; cysteine-rich protein 2; U6 snRNA-associated Sm-like protein LSm7; hypothetical protein FLJ10707; Homo sapiens, Similar to RIKEN cDNA 2310012N15 gene, clone IMAGE:3342825, mRNA, partial cds; macrophage migration inhibitory factor (glycosylation-inhibiting factor); ubiquinol-cytochrome c reductase hinge protein; gap junction protein, alpha 1, 43 kD (connexin 43); dihydropyrimidinase-like 3; aquaporin 1 (channel-forming integral protein, 28 kD); protein expressed in thyroid; macrophage myristoylated alanine-rich C kinase substrate; procollagen-lysine, 2-oxoglutarate 5-dioxygenase (lysine hydroxylase, Ehlers-Danlos syndrome type VI); protease, serine, 11 (IGF binding); 24-dehydrocholesterol reductase; collagen, type IV, alpha 2; profilin 1; apolipoprotein D; hyaluronoglucosaminidase 2; hypothetical protein FLJ22678; quiescin Q6; ras homolog gene family, member A; ras homolog gene family, member A; plasminogen activator, urokinase; insulin-like growth factor binding protein 3; uridine phosphorylase; KIAA0638 protein; B7 homolog 3; lamin A/C; lamin A/C; lamin A/C; regulator of G-protein signalling 12; proteasome (prosome, macropain) 26S subunit, non-ATPase, 8; Homo sapiens, Similar to RIKEN cDNA 5730528L13 gene, clone MGC:17337 IMAGE:4213591, mRNA, complete cds; prosaposin (variant Gaucher disease and variant metachromatic leukodystrophy); laminin, alpha 4; transcription elongation factor A (SII), 1; lectin, galactoside-binding, soluble, 3 binding protein; ribosomal protein S16; glycophorin C (Gerbich blood group); endothelin receptor type B; serine (or cysteine) proteinase inhibitor, Glade E (nexin, plasminogen activator inhibitor type 1), member 1; biglycan; small nuclear ribonucleoprotein polypeptide B″; transmembrane 4 superfamily member 2; TAF11 RNA polymerase II, TATA box binding protein (TBP)-associated factor, 28 kD; lysyl oxidase-like 2; SRY (sex determining region Y)-box 4; SOX4 SRY (sex determining region Y)-box 4; SRY (sex determining region Y)-box 4; actin related protein 2/3 complex, subunit 2 (34 kD); Homo sapiens cDNA: FLJ23507 fis, clone LNG03128; hypothetical protein FLJ12442; Fas (TNFRSF6)-associated via death domain; mitogen-activated protein kinase kinase kinase 11; TEK tyrosine kinase, endothelial (venous malformations, multiple cutaneous and mucosal); insulin receptor; cell membrane glycoprotein, 110000M(r) (surface antigen); Homo sapiens cDNA FLJ11863 fis, clone HEMBA1006926; jagged 1 (Alagille syndrome); KIAA0304 gene product; pre-B-cell leukemia transcription factor 2; Homo sapiens cDNA FLJ31238 fis, clone KIDNE2004864; p53-induced protein; complement component 1, q subcomponent, receptor 1; complement component 1, q subcomponent, receptor 1; apolipoprotein E; chemokine (C—C motif) ligand 3; coagulation factor II (thrombin) receptor-like 3; coagulation factor III (thromboplastin, tissue factor); collagen, type I, alpha 1; collagen, type III, alpha 1 (Ehlers-Danlos syndrome type IV, autosomal dominant); C-type (calcium dependent, carbohydrate-recognition domain) lectin, superfamily member 9; cystatin C (amyloid angiopathy and cerebral hemorrhage); endoplasmic reticulum associated protein 140 kDa; ESTs; ESTs; ESTs, Highly similar to hypothetical protein FLJ10350 [Homo sapiens] [H. sapiens]; ESTs, Highly similar to ITB1_HUMAN Integrin beta-1 precursor (Fibronectin receptor beta subunit) (CD29) (Integrin VLA-4 beta subunit) [H. sapiens]; ESTs, Weakly similar to hypothetical protein FLJ20489 [Homo sapiens] [H. sapiens]; ESTs, Weakly similar to T17346 hypothetical protein DKFZp586O1624.1—human (fragment) [H. sapiens]; ESTs, Weakly similar to T21371 hypothetical protein F25H8.3—Caenorhabditis elegans [C. elegans]; eukaryotic translation initiation factor 4A, isoform 1; heme oxygenase (decycling) 1; Hermansky-Pudlak syndrome 4; Homo sapiens cDNA FLJ34888 fis, clone NT2NE2017332; Homo sapiens cDNA FLJ39848 fis, clone SPLEN2014669; Homo sapiens mRNA full length insert cDNA clone EUROIMAGE 1977059; Homo sapiens, clone IMAGE:4845226, mRNA; hypothetical protein FLJ22329; hypothetical protein FLJ32205; hypothetical protein MGC4677; inhibin, beta B (activin AB beta polypeptide); insulin-like growth factor binding protein 5; junction plakoglobin; KIAA0620 protein; KIAA0943 protein; likely ortholog of rat vacuole membrane protein 1; Lysosomal-associated multispanning membrane protein-5; major histocompatibility complex, class I, B; major histocompatibility complex, class I, C; matrix Gla protein; matrix metalloproteinase 1 (interstitial collagenase); microtubule-associated protein 1 light chain 3 beta; nerve growth factor receptor (TNFR superfamily, member 16); ribosomal protein S9; ring finger protein 40; 5100 calcium binding protein, beta (neural); sema domain, transmembrane domain (TM), and cytoplasmic domain, (semaphorin) 6B; SPARC-like 1 (mast9, hevin); tumor necrosis factor, alpha-induced protein 3; UDP-Gal:betaGlcNAc beta 1,4-galactosyltransferase, polypeptide 3; UDP-GlcNAc:betaGal beta-1,3-N-acetylglucosaminyltransferase 5; von Willebrand factor; v-akt murine thymoma vial oncogene homolog 2; cyclin-dependent kinase (cdc2-like) 10; ortholog mouse myocytic induction/differentiation originator; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16); insulin-like growth factor binding protein; leukemia inhibitory factor; protein tyrosine phosphatase, nonreceptor type I; and Homo sapiens, clone IMAGE:3908182, mRNA, partial cds;
monitoring an expression product of the at least one gene; and
identifying the test compound as a potential anti-cancer drug if it decreases the expression of the at least one gene.

23. The method of claim 22 wherein the cell is a human cell.

24. The method of claim 22 wherein the cell is a glioma cell.

25. The method of claim 22 wherein the cell is a human glioma cell.

26. The method of claim 22 wherein the expression product is RNA.

27. The method of claim 22 wherein the expression product is protein.

28. The method of claim 22 wherein the cell overexpresses the at least one gene relative to a normal cell of the same tissue.

29. The method of claim 22 wherein expression of at least two of said genes is monitored.

30. The method of claim 22 wherein expression of at least three of said genes is monitored.

31. The method of claim 22 wherein expression of at least four of said genes is monitored.

32. The method of claim 22 wherein the test compound is identified if the decrease in expression is at least 50%.

33. The method of claim 22 wherein the test compound is identified if the decrease in expression is at least 80%.

34. The method of claim 22 wherein the decrease in expression is at least 90%.

35. The method of claim 22 wherein the test compound is identified as an anti-glioma drug.

36. A method to aid in diagnosing glioma, comprising the steps of:
detecting an mRNA of at least one gene in a first brain tissue sample suspected of being neoplastic wherein said at least one gene is identified by a tag selected from the group consisting of SEQ ID NO: 1-32; and
comparing expression of the at least one gene in the first brain tissue sample with expression of the at least one gene in a second brain tissue sample which is normal, wherein increased expression of the at least one gene in the first brain tissue sample relative to the second tissue sample identifies the first brain tissue sample as likely to be neoplastic.

37. The method of claim 36 wherein the increased expression of the at least one gene in the first brain tissue sample relative to the second tissue sample is at least two-fold higher.

38. The method of claim 36 wherein the increased expression of the at least one gene in the first brain tissue sample relative to the second tissue sample is at least five-fold higher.

39. The method of claim 36 wherein the increased expression of the at least one gene in the first brain tissue sample relative to the second tissue sample is at least ten-fold higher.

40. The method of claim 36 wherein the first and second tissue samples are from a human.

41. The method of claim 36 wherein the first and second tissue samples are from the same human.

42. The method of claim 36 wherein the step of detecting is performed using a Western blot.

43. The method of claim 36 wherein the step of detecting is performed using an immunoassay.

44. The method of claim 36 wherein the step of detecting is performed using an immunohistochemical assay.

45. The method of claim 36 wherein the step of detecting is performed using SAGE.

46. The method of claim 36 wherein the step of detecting is performed using hybridization to a microarray.

47. A method of identifying a test compound as a potential anti-cancer or anti-glioma drug, comprising the step of:
contacting a test compound with a cell which expresses an mRNA of at least one gene identified by a tag selected from the group consisting of SEQ ID NO: 1-32;
monitoring an mRNA of the at least one gene; and
identifying the test compound as a potential anti-cancer drug if it decreases the expression of the at least one gene.

48. The method of claim 47 wherein the cell is a human cell.

49. The method of claim 47 wherein the cell is a glioma cell.

50. The method of claim 47 wherein the cell is a human glioma cell.

51. The method of claim 47 wherein the expression product is RNA.

52. The method of claim 47 wherein the expression product is protein.

53. The method of claim 47 wherein the cell overexpresses the at least one gene relative to a normal cell of the same tissue.

54. The method of claim 47 wherein expression of at least two of said genes is monitored.

55. The method of claim 47 wherein expression of at least three of said genes is monitored.

56. The method of claim 47 wherein expression of at least four of said genes is monitored.

57. The method of claim 47 wherein the test compound is identified if the decrease in expression is at least 50%.

58. The method of claim 47 wherein the test compound is identified if the decrease in expression is at least 80%.

59. The method of claim 47 wherein the decrease in expression is at least 90%.

60. The method of claim 47 wherein the test compound is identified as an anti-glioma drug.

61. A method to induce an immune response to glioma, comprising:
administering to a mammal a protein or nucleic acid encoding a protein selected from the group consisting of: signal sequence receptor, delta (translocon-associated protein delta); DC2 protein; KIAA0404 protein; symplekin; Huntingtin interacting protein I; plasmalemma vesicle associated protein; KIAA0726 gene product; latexin protein; transforming growth factor, beta 1; hypothetical protein FLJ22215; Rag C protein; hypothetical protein FLJ23471; N-myristoyltransferase 1; hypothetical protein dJ1181N3.1; ribosomal protein L27; Hs 111988; Hs 112238; laminin, alpha 5; protective protein for beta-galactosidase (galactosialidosis); Melanoma associated gene; Melanoma associated gene; E3 ubiquitin ligase SMURF1; collagen, type IV, alpha 1; collagen, type IV, alpha 1; collagen, type IV, alpha 1; insulin-like growth factor binding protein 7; gene predicted from cDNA with a complete coding sequence; Thy-1 cell surface antigen; Hs 127824; GTP binding protein 2; Homo sapiens mRNA; cDNA DKFZp586D0918 (from clone DKFZp586D0918); cutaneous T-cell lymphoma-associated tumor antigen se20-4; differentially expressed nucleolar TGF-beta1 target protein (DENTT); dysferlin, limb girdle muscular dystrophy 2B (autosomal recessive); smoothelin; integrin, alpha 5 (fibronectin receptor, alpha polypeptide); putative translation initiation factor; retinoic acid induced 14; matrix metalloproteinase 9 (gelatinase B, 92 kD gelatinase, 92 kD type IV collagenase); Lutheran blood group (Auberger b antigen included); stanniocalcin 2; nuclear factor (erythroid-derived 2)-like 2; protein tyrosine phosphatase, non-receptor type 1; integrin, alpha 10; collagen, type VI, alpha 2; chromosome 21 open reading frame 25; CDC37 (cell division cycle 37, S. cerevisiae, homolog); Hs 16450; Rho guanine nucleotide exchange factor (GEF) 7; creatine kinase, brain; hypothetical protein FLJ10297; hypothetical protein FLJ10350; TNF-induced protein; tumor necrosis factor receptor superfamily, member 12 (translocating chain-association membrane protein); cofilin 1 (non-muscle); splicing factor proline/glutamine rich (polypyrimidine tract-binding protein-associated); splicing factor proline/glutamine rich (polypyrimidine tract-binding protein-associated); v-ets avian erythroblastosis virus E26 oncogene homolog 1; protease, cysteine, 1 (legumain); ribosomal protein L13; chromosome 22 open reading frame 5; zinc finger protein 144 (MeI-18); degenerative spermatocyte (homolog Drosophila; lipid desaturase); eukaryotic translation initiation factor 2C, 2; mitochondrial ribosomal protein L45; prostate tumor over expressed gene 1; NADH dehydrogenase (ubiquinone) 1 alpha subcomplex, 7 (14.5 kD, B14.5a); glioma endothelial marker 1 precursor; NS1-binding protein; ribosomal protein L38; tuftelin-interacting protein; HLA class II region expressed gene KE2; translocase of inner mitochondrial membrane 17 homolog A (yeast); sudD (suppressor of bimD6, Aspergillus nidulans) homolog; heparan sulfate proteoglycan 2 (perlecan); SEC24 (S. cerevisiae) related gene family, member A; NADH dehydrogenase (ubiquinone) Fe—S protein 7 (20 kD) (NADH-coenzyme Q reductase); DNA segment on chromosome X and Y (unique) 155 expressed sequence; annexin A2; Homo sapiens clone 24670 mRNA sequence; matrix metalloproteinase 10 (stromelysin 2); KIAA1049 protein; G protein-coupled receptor; hypothetical protein FLJ20401; matrix metalloproteinase 14 (membrane-inserted); KIAA0470 gene product; solute carrier family 29 (nucleoside transporters), member 1; stanniocalcin 1; stanniocalcin 1; stanniocalcin 1; tumor suppressor deleted in oral cancer-related 1; tumor suppressor deleted in oral cancer-related 1; apolipoprotein C—I; glutathione peroxidase 4 (phospholipid hydroperoxidase); Hs 272106; transcription factor binding to IGHM enhancer 3; hypothetical protein DKFZp762A227; hypothetical protein FLJ22362; CD59 antigen p18-20 (antigen identified by monoclonal antibodies 16.3A5, EJ16, EJ30, EL32 and G344); PRO0628 protein; melanoma-associated antigen recognised by cytotoxic T lymphocytes; LOC88745; Homo sapiens beta-1,3-galactosyltransferase-6 (B3GALT6) mRNA, complete cds; sprouty (Drosophila) homolog 4; sprouty (Drosophila) homolog 4; Homo sapiens mRNA; cDNA DKFZp434E1515 (from clone DKFZp434E1515); coactosin-like protein; hypothetical protein FLJ21865; Hs296234; KIAA0685 gene product; hypothetical protein FLJ10980; ribosomal protein L10; ribosomal protein S19; Hs 299251; Huntingtin interacting protein K; Homo sapiens mRNA full length insert cDNA clone EUROIMAGE 50374; Hs 311780; Hs 212191; v-akt murine thymoma viral oncogene homolog 2; Hs 328774; transducin-like enhancer of split 2, homolog of Drosophila E(sp1); KIAA1870 protein; ribosomal protein L10a; peptidylprolyl isomerase A (cyclophilin A); Hs 344224; hypothetical protein FLJ23239; hypothetical protein DKFZp761H221; KIAA1887 protein; Homo sapiens mRNA full length insert cDNA clone EUROIMAGE 701679; Homo sapiens cDNA FLJ30634 fis, clone CTONG2002453; Homo sapiens cDNA FLJ32203 fis, clone PLACE6003038, weakly similar to ZINC FINGER PROTEIN 84; Homo sapiens mRNA full length insert cDNA clone EUROIMAGE 1035904; hypothetical protein L0057333; myosin ID; plexin B2; lectin, galactoside-binding, soluble, 8 (galectin 8); double ring-finger protein, Dorfin; DKFZP434B168 protein; LIM domain binding 2; integrin beta 4 binding protein; synaptopodin; Hs 54828; insulin induced gene 1; acetyl LDL receptor; SREC; excision repair cross-complementing rodent repair deficiency, complementation group 1 (includes overlapping antisense sequence); hypothetical protein FLJ22329; schwannomin-interacting protein 1; PTEN induced putative kinase 1; myosin X; Homo sapiens cDNA FLJ32424 fis, clone SKMUS2000954, moderately similar to Homo sapiens F-box protein Fbx25 (FBX25) 97; golgi phosphoprotein 1; splicing factor, arginine/serine-rich 6; laminin, gamma 3; cysteine-rich protein 2; U6 snRNA-associated Sm-like protein LSm7; hypothetical protein FLJ10707; Homo sapiens, Similar to RIKEN cDNA 2310012N15 gene, clone IMAGE:3342825, mRNA, partial cds; macrophage migration inhibitory factor (glycosylation-inhibiting factor); ubiquinol-cytochrome c reductase hinge protein; gap junction protein, alpha 1, 43 kD) (connexin 43); dihydropyrimidinase-like 3; aquaporin 1 (channel-forming integral protein, 28 kD); protein expressed in thyroid; macrophage myristoylated alanine-rich C kinase substrate; procollagen-lysine, 2-oxoglutarate 5-dioxygenase (lysine hydroxylase, Ehlers-Danlos syndrome type VI); protease, serine, 11 (IGF binding); 24-dehydrocholesterol reductase; collagen, type IV, alpha 2; profilin 1; apolipoprotein D; hyaluronoglucosaminidase 2; hypothetical protein FLJ22678; quiescin Q6; ras homolog gene family, member A; ras homolog gene family, member A; plasminogen activator, urokinase; insulin-like growth factor binding protein 3; uridine phosphorylase; KIAA0638 protein; B7 homolog 3; lamin A/C; lamin A/C; lamin A/C; regulator of G-protein signalling 12; proteasome (prosome, macropain) 26S subunit, non-ATPase, 8; Homo sapiens, Similar to RIKEN cDNA 5730528L13 gene, clone MGC:17337 IMAGE:4213591, mRNA, complete cds; prosaposin (variant Gaucher disease and variant metachromatic leukodystrophy); laminin, alpha 4; transcription elongation factor A (SII), 1; lectin, galactoside-binding, soluble, 3 binding protein; ribosomal protein S16; glycophorin C (Gerbich blood group); endothelin receptor type B; serine (or cysteine) proteinase inhibitor, Glade E (nexin, plasminogen activator inhibitor type 1), member 1; biglycan; small nuclear ribonucleoprotein polypeptide B″; transmembrane 4 superfamily member 2; TAF11 RNA polymerase II, TATA box binding protein (TBP)-associated factor, 28 kD; lysyl oxidase-like 2; SRY (sex determining region Y)-box 4; SOX4 SRY (sex determining region Y)-box 4; SRY (sex determining region Y)-box 4; actin related protein 2/3 complex, subunit 2 (34 kD); Homo sapiens cDNA: FLJ23507 fis, clone LNG03128; hypothetical protein FLJ12442; Fas (TNFRSF6)-associated via death domain; mitogen-activated protein kinase kinase kinase 11; TEK tyrosine kinase, endothelial (venous malformations, multiple cutaneous and mucosal); insulin receptor; cell membrane glycoprotein, 110000M(r) (surface antigen); Homo sapiens cDNA FLJ11863 fis, clone HEMBA1006926; jagged 1 (Alagille syndrome); KIAA0304 gene product; pre-B-cell leukemia transcription factor 2; Homo sapiens cDNA FLJ31238 fis, clone KIDNE2004864; p53-induced protein; complement component 1, q subcomponent, receptor 1; complement component 1, q subcomponent, receptor 1; apolipoprotein E; chemokine (C—C motif) ligand 3; coagulation factor II (thrombin) receptor-like 3; coagulation factor III (thromboplastin, tissue factor); collagen, type I, alpha 1; collagen, type III, alpha 1 (Ehlers-Danlos syndrome type IV, autosomal dominant); C-type (calcium dependent, carbohydrate-recognition domain) lectin, superfamily member 9; cystatin C (amyloid angiopathy and cerebral hemorrhage); endoplasmic reticulum associated protein 140 kDa; ESTs; ESTs; ESTs, Highly similar to hypothetical protein FLJ10350 [Homo sapiens] [H. sapiens]; ESTs, Highly similar to ITB1_HUMAN Integrin beta-1 precursor (Fibronectin receptor beta subunit) (CD29) (Integrin VLA-4 beta subunit) [H. sapiens]; ESTs, Weakly similar to hypothetical protein FLJ20489 [Homo sapiens] [H. sapiens]; ESTs, Weakly similar to T17346 hypothetical protein DKFZp586O1624.1—human (fragment) [H. sapiens]; ESTs, Weakly similar to T21371 hypothetical protein F25H8.3—Caenorhabditis elegans [C. elegans]; eukaryotic translation initiation factor 4A, isoform 1; heme oxygenase (decycling) 1; Hermansky-Pudlak syndrome 4; Homo sapiens cDNA FLJ34888 fis, clone NT2NE2017332; Homo sapiens cDNA FLJ39848 fis, clone SPLEN2014669; Homo sapiens mRNA full length insert cDNA clone EUROIMAGE 1977059; Homo sapiens, clone IMAGE:4845226, mRNA; hypothetical protein FLJ22329; hypothetical protein FLJ32205; hypothetical protein MGC4677; inhibin, beta B (activin AB beta polypeptide); insulin-like growth factor binding protein 5; junction plakoglobin; KIAA0620 protein; KIAA0943 protein; likely ortholog of rat vacuole membrane protein 1; Lysosomal-associated multispanning membrane protein-5; major histocompatibility complex, class I, B; major histocompatibility complex, class I, C; matrix Gla protein; matrix metalloproteinase 1 (interstitial collagenase); microtubule-associated protein 1 light chain 3 beta; nerve growth factor receptor (TNFR superfamily, member 16); ribosomal protein S9; ring finger protein 40; S100 calcium binding protein, beta (neural); sema domain, transmembrane domain (TM), and cytoplasmic domain, (semaphorin) 6B; SPARC-like 1 (mast9, hevin); tumor necrosis factor, alpha-induced protein 3; UDP-Gal:betaGlcNAc beta 1,4-galactosyltransferase, polypeptide 3; UDP-GlcNAc:betaGal beta-1,3-N-acetylglucosaminyltransferase 5; von Willebrand factor; v-akt murine thymoma vial oncogene homolog 2; cyclin-dependent kinase (cdc2-like) 10; ortholog mouse myocytic induction/differentiation originator; brain-specific angiogenesis inhibitor 1; EGF-TM7 latrophilin-related protein; sema domain; integrin, alpha 5; likely ortholog of mouse fibronectin type III; Lutheran blood group (Auberger b antigen included); SSR4, TRAPD; nerve growth factor receptor (TNFR superfamily, member 16); insulin-like growth factor binding protein; leukemia inhibitory factor; protein tyrosine phosphatase, nonreceptor type I; and Homo sapiens, clone IMAGE:3908182, mRNA, partial cds, whereby an immune response to the protein is induced.

62. The method of claim 61 wherein a protein is administered.

63. The method of claim 61 wherein a nucleic acid is administered.

64. The method of claim 63 wherein the nucleic acid is administered intramuscularly.

65. The method of claim 62 further comprising administering an immune adjuvant to the mammal.

66. The method of claim 61 wherein the mammal has a glioma.

67. The method of claim 61 wherein the mammal has had a glioma surgically removed.
